(19) United States
(12) Patent Application Publication
(10) Pub. No.: US 2025/0295815 A1
(43) Pub. Date: Sep. 25, 2025

(54) PORPHYRIN-HYDROPORPHYRIN COMPOUNDS, COMPOSITIONS COMPRISING THE SAME AND METHODS OF USE THEREOF

(71) Applicants: North Carolina State University, Raleigh, NC (US); NIRvana Sciences, Research Triangle Park, NC (US)

(72) Inventors: Jonathan S. Lindsey, Raleigh, NC (US); Christopher MacNevin, Durham, NC (US); Joshua Akhigbe, Durham, NC (US); Michael Gorczynski, Wake Forest, NC (US); Masahiko Taniguchi, Raleigh, NC (US)

(21) Appl. No.: 18/553,881

(22) PCT Filed: Apr. 7, 2022
(86) PCT No.: PCT/US2022/023827

§ 371 Date: Oct. 4, 2023

Related U.S. Application Data
(60) Provisional application No. 63/171,870, filed on Apr. 7, 2021.

(51) Int. Cl. A61K 49/00; C07D 487/22; G01N 33/58

(52) U.S. Cl. CPC A61K 49/0036; C07D 487/22; G01N 33/582

(57) ABSTRACT

Described herein are compounds that include a first porphyrin; and a first hydroporphyrin, wherein the first porphyrin is attached to the first hydroporphyrin. The compound may be a luminescent compound (e.g., a fluorescent compound). Also provided are particles and compositions including compounds described herein. Further provided are methods of making and using the particles and methods of making the same.

### STATEMENT OF GOVERNMENT SUPPORT

This invention was made with government support under grant number AI112302 awarded by the National Institutes of Health. The government has certain rights in the invention.

### FIELD

The present invention relates to compounds such as porphyrin-hydroporphyrin compounds along with compositions comprising the same. The present invention also relates to compounds for use in biomedical applications and methods of using compounds in biomedical applications.

### BACKGROUND

Molecules that absorb or emit ultraviolet, visible or near-infrared (NIR) light (chromophores) are used for a variety of biomedical and related applications, such as flow cytometry, molecular optical imaging, and photodynamic therapy. As the applications continue to broaden and become refined, the desire for specific photophysical properties is becoming more acute. Relevant photophysical properties include absorption wavelengths, emission (fluorescence or phosphorescence) wavelengths, spacing between absorption and fluorescence wavelengths, fluorescence lifetime, fluorescence quantum yield, triplet lifetime, and triplet yield. Another relevant electronic characteristic is the redox properties of the molecules, which control charge transfer reactions that are unwanted for typical applications. For most chromophores, it is often very difficult to obtain a suitably large spacing between the longest wavelength absorption feature and the shortest-wavelength fluorescence feature (the so-called “Stokes shift”). However, for many biomedical applications, it is desirable, and in some cases, essential, to have independent control over two or more of these photophysical properties in order to meet the fundamental or technical demands of the application.

For example, there is a need in biomedical diagnostics for fluorescent reagents that can be excited at a common wavelength with detection of their fluorescence emission at multiple and different distinct wavelengths. In general, this is known as a “multiplex” assay and is exemplified by flow cytometry. Hydroporphyrins, such as chlorins and bacteriochlorins, can exhibit distinct narrow emission profiles. However, a 405 nm laser is often used in flow cytometry and hydroporphyrins may not have optimal 405 nm fluorescence excitation, which can result in weak absorption and low brightness, thereby limiting their usefulness particularly for multiplex assays.

### SUMMARY OF THE INVENTION

Tunability and independent control of key photophysical properties of molecules for biomedical and other applications may be facilitated by the use of compounds of the present invention such as dyads comprising two distinct chromophores (e.g., a donor and an acceptor) that are joined, optionally via a linking group. Compounds of the present invention can allow for relatively rapid and efficient energy transfer from one chromophore (e.g., a donor chromophore) to another chromophore (e.g., an acceptor chromophore). A donor chromophore may be chosen for absorption attributes and an acceptor chromophore may be chosen for emission attributes. By allowing for efficient energy transfer from the donor to the acceptor, the absorption and emission features of the chromophores of a compound of the present invention can provide the ability to design and/or tune a compound to have desired spectral features.

Provided according to embodiments of the invention is a compound that includes a first porphyrin and a first hydroporphyrin, wherein the first porphyrin is attached to the first hydroporphyrin. In some embodiments, a compound of the present invention is a luminescent compound (e.g., a fluorescent compound) that includes a first porphyrin and a first hydroporphyrin, wherein the first porphyrin is attached to the first hydroporphyrin. In some embodiments, the luminescent compound is a fluorescent compound. In some embodiments, the first porphyrin is attached to the first hydroporphyrin via a linking group. In some embodiments, the first hydroporphyrin is a chlorin. In some embodiments, the first hydroporphyrin is a bacteriochlorin and, in some embodiments, may be an isobacteriochlorin or an azabacteriochlorin. It is noted that aspects of the invention described with respect to one embodiment, may be incorporated in a different embodiment although not specifically described relative thereto. That is, all embodiments and/or features of any embodiment can be combined in any way and/or combination. Applicant reserves the right to change any originally filed claim and/or file any new claim accordingly, including the right to be able to amend any originally filed claim to depend from and/or incorporate any feature of any other claim or claims although not originally claimed in that manner. These and other objects and/or aspects of the present invention are explained in detail in the specification set forth below. Further features, advantages and details of the present invention will be appreciated by those of ordinary skill in the art from a reading of the figures and the detailed description of the preferred embodiments that follow, such description being merely illustrative of the present invention.

### BRIEF DESCRIPTION OF THE DRAWINGS

FIG. 1 is a schematic illustration of an exemplary compound of the present invention that includes a donor and an acceptor that are joined by a linker according to some embodiments.

FIGS. 2-4 show exemplary intermediates that may be used in forming a compound of the present invention according to some embodiments.

FIGS. 5-8 show fluorescence spectrum for exemplary compounds of the present invention.

### DETAILED DESCRIPTION OF THE EXAMPLE EMBODIMENTS

The present invention will now be described more fully hereinafter. This invention may, however, be embodied in different forms and should not be construed as limited to the embodiments set forth herein. Rather, these embodiments are provided so that this disclosure will be thorough and complete, and will fully convey the scope of the invention to those skilled in the art.

The terminology used in the description of the invention herein is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. The terminology used in the description of the invention herein is for the purpose of describing particular embodiments only and is not intended to be limiting of the invention. All publications, patent applications, patents and other references mentioned herein are incorporated by reference in their entirety. In case of a conflict in terminology, the present specification is controlling.

Also as used herein, “and/or” refers to and encompasses any and all possible combinations of one or more of the associated listed items, as well as the lack of combinations when interpreted in the alternative (“or”).

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed.

As used herein, the transitional phrase “consisting essentially of” (and grammatical variants) is to be interpreted as encompassing the recited materials or steps “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP § 2111.03. Thus, the term “consisting essentially of” as used herein should not be interpreted as equivalent to “comprising.”

It will be understood that although the terms “first,” “second,” etc. may be used herein to describe various elements, these elements should not be limited by these terms. These terms are only used to distinguish one element from another. Thus, a “first” element could be termed a “second” element without departing from the teachings of the present embodiments.

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measurable value may include any other range and/or individual value therein.

“Halo” as used herein refers to any suitable halogen, including —F, —Cl, —Br, and —I.

“Mercapto” as used herein refers to an —SH group.

“Azido” as used herein refers to an —N₃ group.

“Cyano” as used herein refers to a —CN group.

“Hydroxyl” as used herein refers to an —OH group.

“Nitro” as used herein refers to an —NO₂ group.

“Alkyl” as used herein alone or as part of another group, refers to a straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, and the like. “Loweralkyl” as used herein, is a subset of alkyl, and, in some embodiments, refers to a straight or branched chain hydrocarbon group containing from 1 to 4 carbon atoms. Representative examples of loweralkyl include, but are not limited to, methyl, ethyl, n-propyl, iso-propyl, n-butyl, iso-butyl, tert-butyl, and the like. The term “alkyl” or “loweralkyl” is intended to include both substituted and unsubstituted alkyl or loweralkyl unless otherwise indicated and these groups may be substituted with groups selected from halo, alkyl, haloalkyl, alkenyl, alkynyl, cycloalkyl, cycloalkylalkyl, aryl, arylalkyl, heterocyclo, heterocycloalkyl, hydroxyl, alkoxy (thereby creating a polyalkoxy such as polyethylene glycol), alkenyloxy, alkynyloxy, haloalkoxy, cycloalkoxy, cycloalkylalkyloxy, aryloxy, arylalkyloxy, heterocyclooxy, heterocycloalkyloxy, mercapto, alkyl-S(O)ₘ, haloalkyl-S(O)ₘ, alkenyl-S(O)ₘ, alkynyl-S(O)ₘ, cycloalkyl-S(O)ₘ, cycloalkylalkyl-S(O)ₘ, aryl-S(O)ₘ, arylalkyl-S(O)ₘ, heterocyclo-S(O)ₘ, heterocycloalkyl-S(O)ₘ, amino, carboxy, alkylamino, alkenylamino, alkynylamino, haloalkylamino, cycloalkylamino, cycloalkylalkylamino, arylamino, arylalkylamino, heterocycloamino, heterocycloalkylamino, disubstituted-amino, acylamino, acyloxy, ester, amide, sulfonamide, urea, alkoxyacylamino, aminoacyloxy, nitro or cyano where m=0, 1, 2 or 3.

“Alkenyl” as used herein alone or as part of another group, refers to a straight or branched chain hydrocarbon containing from 1 to 10 carbon atoms (or in loweralkenyl 1 to 4 carbon atoms) which include 1 to 4 double bonds in the normal chain. Representative examples of alkenyl include, but are not limited to, vinyl, 2-propenyl, 3-butenyl, 2-butenyl, 4-pentenyl, 3-pentenyl, 2-hexenyl, 3-hexenyl, 2,4-heptadiene, and the like. The term “alkenyl” or “loweralkenyl” is intended to include both substituted and unsubstituted alkenyl or loweralkenyl unless otherwise indicated and these groups may be substituted with groups as described in connection with alkyl and loweralkyl above.

“Alkynyl” as used herein alone or as part of another group, refers to a straight or branched chain hydrocarbon containing from 1 to 10 carbon atoms (or in loweralkynyl 1 to 4 carbon atoms) which include 1 triple bond in the normal chain. Representative examples of alkynyl include, but are not limited to, 2-propynyl, 3-butynyl, 2-butynyl, 4-pentynyl, 3-pentynyl, and the like. The term “alkynyl” or “loweralkynyl” is intended to include both substituted and unsubstituted alkynyl or loweralkynyl unless otherwise indicated and these groups may be substituted with the same groups as set forth in connection with alkyl and loweralkyl above.

“Alkoxy” as used herein alone or as part of another group, refers to an alkyl or loweralkyl group, as defined herein (and thus including substituted versions such as polyalkoxy), appended to the parent molecular moiety through an oxy group, —O—. Representative examples of alkoxy include, but are not limited to, methoxy, ethoxy, propoxy, 2-propoxy, butoxy, tert-butoxy, pentyloxy, hexyloxy and the like.

“Acyl” as used herein alone or as part of another group refers to a —C(O)R radical, where R is any suitable substituent such as aryl, alkyl, alkenyl, alkynyl, cycloalkyl or other suitable substituent as described herein.

“Haloalkyl” as used herein alone or as part of another group, refers to at least one halogen, as defined herein, appended to the parent molecular moiety through an alkyl group, as defined herein. Representative examples of haloalkyl include, but are not limited to, chloromethyl, 2-fluoroethyl, trifluoromethyl, pentafluoroethyl, 2-chloro-3-fluoropentyl, and the like.

“Alkylthio” as used herein alone or as part of another group, refers to an alkyl group, as defined herein, appended to the parent molecular moiety through a thio moiety, as defined herein. Representative examples of alkylthio include, but are not limited, methylthio, ethylthio, tert-butylthio, hexylthio, and the like.

“Aryl” as used herein alone or as part of another group, refers to a monocyclic carbocyclic ring system or a bicyclic carbocyclic fused ring system having one or more aromatic rings. Representative examples of aryl include, but are not limited to, azulenyl, indanyl, indenyl, naphthyl, phenyl, tetrahydronaphthyl, and the like. The term “aryl” is intended to include both substituted and unsubstituted aryl unless otherwise indicated and these groups may be substituted with the same groups as set forth in connection with alkyl and loweralkyl above.

“Arylalkyl” as used herein alone or as part of another group, refers to an aryl group, as defined herein, appended to the parent molecular moiety through an alkyl group, as defined herein. Representative examples of arylalkyl include, but are not limited to, benzyl, 2-phenylethyl, 3-phenylpropyl, 2-naphth-2-ylethyl, and the like.

“Amino” as used herein means the radical —NH2.

“Alkylamino” as used herein alone or as part of another group means the radical —NHR, where R is an alkyl group.

“Arylalkylamino” as used herein alone or as part of another group means the radical —NHR, where R is an arylalkyl group.

“Disubstituted-amino” as used herein alone or as part of another group means the radical —NRₐR_{b}, where Rₐ and R_{b} are independently selected from the groups alkyl, haloalkyl, alkenyl, alkynyl, cycloalkyl, cycloalkylalkyl, aryl, arylalkyl, heterocyclo, heterocycloalkyl.

“Acylamino” as used herein alone or as part of another group means the radical —NRₐR_{b}, where Rₐ is an acyl group as defined herein and R_{b} is selected from the groups hydrogen, alkyl, haloalkyl, alkenyl, alkynyl, cycloalkyl, cycloalkylalkyl, aryl, arylalkyl, heterocyclo, heterocycloalkyl.

“Acyloxy” as used herein alone or as part of another group means the radical —OR, where R is an acyl group as defined herein.

“Ester” as used herein alone or as part of another group refers to a —C(O)OR radical, where R is any suitable substituent such as alkyl, cycloalkyl, alkenyl, alkynyl or aryl.

“Formyl” as used herein refers to a —C(O)H group.

“Carboxylic acid” as used herein refers to a —C(O)OH group.

“Sulfoxyl” as used herein refers to a compound of the formula —S(O)R, where R is any suitable substituent such as alkyl, cycloalkyl, alkenyl, alkynyl or aryl.

“Sulfonyl as used herein refers to a compound of the formula —S(O)(O)R, where R is any suitable substituent such as alkyl, cycloalkyl, alkenyl, alkynyl or aryl.

“Sulfonate” as used herein refers to a compound of the formula —S(O)(O)OR, where R is any suitable substituent such as alkyl, cycloalkyl, alkenyl, alkynyl or aryl.

“Sulfonic acid as used herein refers to a compound of the formula —S(O)(O)OH.

“Amide” as used herein alone or as part of another group refers to a —C(O)NRₐR_{b} radical, where Rₐ and R_{b} are any suitable substituent such as alkyl, cycloalkyl, alkenyl, alkynyl or aryl.

“Sulfonamide” as used herein alone or as part of another group refers to a —S(O)₂NRₐR_{b} radical, where Rₐ and R_{b} are any suitable substituent such as H, alkyl, cycloalkyl, alkenyl, alkynyl or aryl.

“Urea” as used herein alone or as part of another group refers to an —N(R_{c})C(O)NRₐR_{b} radical, where Rₐ, R_{b} and Re are any suitable substituent such as H, alkyl, cycloalkyl, alkenyl, alkynyl or aryl.

“Alkoxyacylamino” as used herein alone or as part of another group refers to an —N(Rₐ)C(O)OR_{b} radical, where Rₐ, R_{b} are any suitable substituent such as H, alkyl, cycloalkyl, alkenyl, alkynyl or aryl.

“Aminoacyloxy” as used herein alone or as part of another group refers to an —OC(O)NRₐR_{b} radical, where Rₐ and R_{b} are any suitable substituent such as H, alkyl, cycloalkyl, alkenyl, alkynyl or aryl.

“Cycloalkyl” as used herein alone or as part of another group, refers to a saturated or partially unsaturated cyclic hydrocarbon group containing from 3, 4 or 5 to 6, 7 or 8 carbons (which carbons may be replaced in a heterocyclic group as discussed below). Representative examples of cycloalkyl include, cyclopropyl, cyclobutyl, cyclopentyl, cyclohexyl, cycloheptyl, and cyclooctyl. These rings may be optionally substituted with additional substituents as described herein such as halo or loweralkyl. The term “cycloalkyl” is generic and intended to include heterocyclic groups as discussed below unless specified otherwise.

“Heterocyclic group” or “heterocyclo” as used herein alone or as part of another group, refers to an aliphatic (e.g., fully or partially saturated heterocyclo) or aromatic (e.g., heteroaryl) monocyclic- or a bicyclic-ring system. Monocyclic ring systems are exemplified by any 5 or 6 membered ring containing 1, 2, 3, or 4 heteroatoms independently selected from oxygen, nitrogen and sulfur. The 5 membered ring has from 0-2 double bonds and the 6 membered ring has from 0-3 double bonds. Representative examples of monocyclic ring systems include, but are not limited to, azetidine, azepine, aziridine, diazepine, 1,3-dioxolane, dioxane, dithiane, furan, imidazole, imidazoline, imidazolidine, isothiazole, isothiazoline, isothiazolidine, isoxazole, isoxazoline, isoxazolidine, morpholine, oxadiazole, oxadiazoline, oxadiazolidine, oxazole, oxazoline, oxazolidine, piperazine, piperidine, pyran, pyrazine, pyrazole, pyrazoline, pyrazolidine, pyridine, pyrimidine, pyridazine, pyrrole, pyrroline, pyrrolidine, tetrahydrofuran, tetrahydrothiophene, tetrazine, tetrazole, thiadiazole, thiadiazoline, thiadiazolidine, thiazole, thiazoline, thiazolidine, thiophene, thiomorpholine, thiomorpholine sulfone, thiopyran, triazine, triazole, trithiane, and the like. Bicyclic ring systems are exemplified by any of the above monocyclic ring systems fused to an aryl group as defined herein, a cycloalkyl group as defined herein, or another monocyclic ring system as defined herein. Representative examples of bicyclic ring systems include but are not limited to, for example, benzimidazole, benzothiazole, benzothiadiazole, benzothiophene, benzoxadiazole, benzoxazole, benzofuran, benzopyran, benzothiopyran, benzodioxine, 1,3-benzodioxole, cinnoline, indazole, indole, indoline, indolizine, naphthyridine, isobenzofuran, isobenzothiophene, isoindole, isoindoline, isoquinoline, phthalazine, purine, pyranopyridine, quinoline, quinolizine, quinoxaline, quinazoline, tetrahydroisoquinoline, tetrahydroquinoline, thiopyranopyridine, and the like. These rings include quaternized derivatives thereof and may be optionally substituted with groups selected from halo, alkyl, haloalkyl, alkenyl, alkynyl, cycloalkyl, cycloalkylalkyl, aryl, arylalkyl, heterocyclo, heterocycloalkyl, hydroxyl, alkoxy, alkenyloxy, alkynyloxy, haloalkoxy, cycloalkoxy, cycloalkylalkyloxy, aryloxy, arylalkyloxy, heterocyclooxy, heterocycloalkyloxy, mercapto, alkyl-S(O)ₘ, haloalkyl-S(O)ₘ, alkenyl-S(O)ₘ, alkynyl-S(O)ₘ, cycloalkyl-S(O)ₘ, cycloalkylalkyl-S(O)ₘ, aryl-S(O)ₘ, arylalkyl-S(O)ₘ, heterocyclo-S(O)ₘ, heterocycloalkyl-S(O)ₘ, amino, alkylamino, alkenylamino, alkynylamino, haloalkylamino, cycloalkylamino, cycloalkylalkylamino, arylamino, arylalkylamino, heterocycloamino, heterocycloalkylamino, disubstituted-amino, acylamino, acyloxy, ester, amide, sulfonamide, urea, alkoxyacylamino, aminoacyloxy, nitro or cyano where m=0, 1, 2 or 3. In some embodiments, the heterocyclo group includes pyridyl and/or imidazolyl groups, these terms including the quaternized derivatives thereof, including but not limited to quaternary pyridyl and imidazolyl groups, examples of which include but are not limited to:

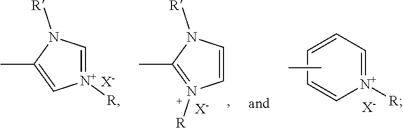

where R and R′ are each a suitable substituent as described in connection with “alkyl” above, and particularly alkyl (such as methyl, ethyl or propyl), arylalkyl (such as benzyl), optionally substituted with hydroxy (—OH), phosphonic acid (—PO₃H₂) or sulfonic acid (—SO₃H), and X⁻ is a counterion.

“Spiroalkyl” as used herein alone or as part of another group, refers to a straight or branched chain hydrocarbon, saturated or unsaturated, containing from 3 to 8 carbon atoms. Representative examples include, but are not limited to, —CH₂CH₂CH₂—, —CH₂CH₂CH₂CH₂—, —CH₂CH₂CH₂CH₂CH₂—, —CH₂CH₂CHCHCH₂—, —CH₂CH₂CH₂CH₂CH₂CH₂—, etc. The term “spiroalkyl” is intended to include both substituted and unsubstituted “spiroalkyl” unless otherwise indicated and these groups may be substituted with groups selected from halo, alkyl, haloalkyl, alkenyl, alkynyl, cycloalkyl, cycloalkylalkyl, aryl, arylalkyl, heterocyclo, heterocycloalkyl, hydroxyl, alkoxy, alkenyloxy, alkynyloxy, haloalkoxy, cycloalkoxy, cycloalkylalkyloxy, aryloxy, arylalkyloxy, heterocyclooxy, heterocycloalkyloxy, mercapto, alkyl-S(O)ₘ, haloalkyl-S(O)ₘ, alkenyl-S(O)ₘ, alkynyl-S(O)ₘ, cycloalkyl-S(O)ₘ, cycloalkylalkyl-S(O)ₘ, aryl-S(O)ₘ, arylalkyl-S(O)ₘ, heterocyclo-S(O)ₘ, heterocycloalkyl-S(O)ₘ, amino, alkylamino, alkenylamino, alkynylamino, haloalkylamino, cycloalkylamino, cycloalkylalkylamino, arylamino, arylalkylamino, heterocycloamino, heterocycloalkylamino, disubstituted-amino, acylamino, acyloxy, ester, amide, sulfonamide, urea, alkoxyacylamino, aminoacyloxy, nitro or cyano where m=0, 1 or 2.

As used herein, a “targeting group” Targeting groups such as antibodies, proteins, peptides, and nucleic acids may be attached by means of the linking group

“Treatment” as used herein means any manner in which one or more of the symptoms of a disease or disorder are ameliorated or otherwise beneficially altered. Treatment also encompasses any pharmaceutical use of the compositions herein, such as use for treating hyperproliferating tissue or neovascularization mediated diseases or disorders, or diseases or disorders in which hyperproliferating tissue or neovascularization is implicated. As used herein, amelioration of the symptoms of a particular disorder by administration of a particular compound or pharmaceutical composition refers to any lessening, whether permanent or temporary, lasting or transient that can be attributed to or associated with administration of the composition.

“Prodrug” as used herein is a compound that, upon in vivo administration, is metabolized by one or more steps or processes or otherwise converted to the biologically, pharmaceutically or therapeutically active form of the compound.

“Antibody” as used herein refers generally to immunoglobulins or fragments thereof that specifically bind to antigens to form immune complexes. The antibody may be whole immunoglobulin of any class, e.g., IgG, IgM, IgA, IgD, IgE, chimeric or hybrid antibodies with dual or multiple antigen or epitope specificities. It may be a polyclonal antibody, and in some embodiments may be an affinity-purified antibody from a human or an appropriate animal, e.g., a primate, goat, rabbit, mouse or the like. Monoclonal antibodies are also suitable for use in the present invention and may be used because of their high specificities. They are readily prepared by what are now considered conventional procedures of immunization of mammals with immunogenic antigen preparation, fusion of immune lymph or spleen cells with an immortal myeloma cell line, and isolation of specific hybridoma clones. More unconventional methods of preparing monoclonal antibodies are not excluded, such as interspecies fusions and genetic engineering manipulations of hypervariable regions, since it is primarily the antigen specificity of the antibodies that affects their utility. Newer techniques for production of monoclonals can also be used, e.g., human monoclonals, interspecies monoclonals, chimeric (e.g., human/mouse) monoclonals, genetically engineered antibodies and the like.

“Infecting agent” as used herein denotes invading microbes or parasites. As used herein, “microbe” denotes virus, bacteria, rickettsia, mycoplasma, protozoa, fungi and like microorganisms, and “parasite” denotes infectious, generally microscopic or very small multicellular invertebrates, or ova or juvenile forms thereof, which are susceptible to antibody-induced clearance or lytic or phagocytic destruction, e.g., malarial parasites, spirochetes and the like.

“Tumor” as used herein denotes a neoplasm and includes both benign and malignant tumors. This term particularly includes malignant tumors which can be either solid (such as a breast, liver, or prostate carcinoma) or non-solid (such as a leukemia). Tumors can also be further divided into subtypes, such as adenocarcinomas (e.g., of the breast, prostate, or lung).

“Target” as used herein denotes the object that is intended to be detected, diagnosed, impaired or destroyed by the methods provided herein, and includes target cells, target tissues, and target compositions. “Target tissues” and “target cells” as used herein are those tissues that are intended to be impaired or destroyed by this treatment method. Photosensitizing compounds bind to or collect in these target tissues or target cells; then when sufficient radiation is applied, these tissues or cells are impaired or destroyed. Target cells are cells in target tissue, and the target tissue includes, but is not limited to, vascular endothelial tissue, abnormal vascular walls of tumors, solid tumors such as (but not limited to) tumors of the head and neck, tumors of the eye, tumors of the gastrointestinal tract, tumors of the liver, tumors of the breast, tumors of the prostate, tumors of the lung, nonsolid tumors and malignant cells of the hematopoietic and lymphoid tissue, neovascular tissue, other lesions in the vascular system, bone marrow, and tissue or cells related to autoimmune disease. Also included among target cells are cells undergoing substantially more rapid division as compared to non-target cells.

“Non-target tissues” as used herein are all the tissues of the subject which are not intended to be impaired or destroyed by the treatment method. These non-target tissues include but are not limited to healthy blood cells, and other normal tissue, not otherwise identified to be targeted.

“Target compositions” as used herein are those compositions that are intended to be impaired or destroyed by this treatment method, and may include one or more pathogenic agents, including but not limited to bacteria, viruses, fungi, protozoa, and toxins as well as cells and tissues infected or infiltrated therewith. The term “target compositions” also includes, but is not limited to, infectious organic particles such as prions, toxins, peptides, polymers, and other compounds that may be selectively and specifically identified as an organic target that is intended to be impaired or destroyed by this treatment method.

“Hyperproliferative tissue” as used herein means tissue that grows out of control and includes neoplastic tissue, tumors and unbridled vessel growth such as blood vessel growth found in age-related macular degeneration and often occurring after glaucoma surgeries.

“Hyperproliferative disorders” as used herein denotes those conditions disorders sharing as an underlying pathology excessive cell proliferation caused by unregulated or abnormal cell growth and include uncontrolled angiogenesis. Examples of such hyperproliferative disorders include, but are not limited to, cancers or carcinomas, acute and membrano-proliferative glomerulonephritis, myelomas, psoriasis, atherosclerosis, psoriatic arthritis, rheumatoid arthritis, diabetic retinopathies, macular degeneration, corneal neovascularization, choroidal hemangioma, recurrence of pterygii, and scarring from excimer laser surgery and glaucoma filtering surgery.

“Therapeutically effective dose” as used herein is a dose sufficient to prevent advancement, or to cause regression of the disease, or which is capable of relieving symptoms caused by the disease.

“Irradiating” and “irradiation” as used herein includes exposing a subject to all wavelengths of light. In some embodiments, the irradiating wavelength is selected to match the wavelength(s) which excite the photosensitive compound. In some embodiments, the radiation wavelength matches the excitation wavelength of the photosensitive compound and has low absorption by the non-target tissues of the subject, including blood proteins.

Irradiation is further defined herein by its coherence (laser) or non-coherence (non-laser), as well as intensity, duration, and timing with respect to dosing using the photosensitizing compound. The intensity or fluence rate must be sufficient for the light to reach the target tissue. The duration or total fluence dose must be sufficient to photoactivate enough photosensitizing compound to act on the target tissue. Timing with respect to dosing with the photosensitizing compound is important, because 1) the administered photosensitizing compound requires some time to home in on target tissue and 2) the blood level of many photosensitizing compounds decreases with time. The radiation energy is provided by an energy source, such as a laser or cold cathode light source, that is external to the subject, or that is implanted in the subject, or that is introduced into a subject, such as by a catheter, optical fiber or by ingesting the light source in capsule or pill form (e.g., as disclosed in. U.S. Pat. No. 6,273,904 (2001)).

Some embodiments of the present invention are drawn to the use of light energy for administering photodynamic therapy (PDT) to destroy tumors, other forms of energy are within the scope of this invention, as will be understood by those of ordinary skill in the art. Such forms of energy include, but are not limited to: thermal, sonic, ultrasonic, chemical, light, microwave, ionizing (such as x-ray and gamma ray), mechanical, and electrical. For example, sonodynamically induced or activated agents include, but are not limited to: gallium-porphyrin complex (see Yumita et al., Cancer Letters 112: 79-86 (1997)), other porphyrin complexes, such as protoporphyrin and hematoporphyrin (see Umemura et al., Ultrasonics Sonochemistry 3: S187-S191 (1996)); other cancer drugs, such as daunorubicin and adriamycin, used in the presence of ultrasound therapy (see Yumita et al., Japan J. Hyperthermic Oncology 3(2):175-182 (1987)).

“Coupling agent” as used herein, refers to a reagent capable of coupling a photosensitizer to a targeting agent.

“Targeting group” refers to a compound that homes in on and/or associates and/or binds to a particular tissue, receptor, infecting agent or other area of the body of the subject to be treated, such as a target tissue or target composition, such as described above. Examples of a targeting group or agent include but are not limited to an antibody, a ligand (e.g., a drug), one member of a ligand-receptor binding pair, nucleic acids, proteins and peptides, and liposomal suspensions, including tissue-targeted liposomes.

“Specific binding pair” and “ligand-receptor binding pair” as used herein refers to two different molecules, where one of the molecules has an area on the surface or in a cavity which specifically attracts or binds to a particular spatial or polar organization of the other molecule, causing both molecules to have an affinity for each other. The members of the specific binding pair are referred to as ligand and receptor (anti-ligand). The terms ligand and receptor are intended to encompass the entire ligand or receptor or portions thereof sufficient for binding to occur between the ligand and the receptor. Examples of ligand-receptor binding pairs include, but are not limited to, hormones and hormone receptors, for example epidermal growth factor and epidermal growth factor receptor, tumor necrosis factor-α and tumor necrosis factor-receptor, and interferon and interferon receptor; avidin and biotin or antibiotin; antibody and antigen pairs; enzymes and substrates, drug and drug receptor; cell-surface antigen and lectin; two complementary nucleic acid strands; nucleic acid strands and complementary oligonucleotides; interleukin and interleukin receptor; and stimulating factors and their receptors, such as granulocyte-macrophage colony stimulating factor (GMCSF) and GMCSF receptor and macrophage colony stimulating factor (MCSF) and MCSF receptor.

“Biological materials” as used herein refers to both tissues (such as biopsy tissues) and cells, as well as biological fluids such as blood, urine, plasma, cerebrospinal fluid, mucus, sputum, etc.

Subjects to be treated by the methods of the present invention for diagnostic and/or therapeutic purposes include both human subjects and animal subjects (particularly mammalian subjects such as, e.g., dogs, cats, horses, monkeys, chimpanzees, etc.) for veterinary purposes.

Provided according to embodiments of the invention is a compound that includes at least one porphyrin linked to at least one hydroporphyrin. In some embodiments, a compound of the present invention is a luminescent compound. A “luminescent compound” as used herein refers to a compound that can emit light, wherein the compound includes at least one porphyrin linked to at least one hydroporphyrin. For example, a luminescent compound can emit light but the nature of the originating state (e.g., singlet, triplet, and/or another state) for the luminescent compound is not specified. Exemplary luminescent compounds include, but are not limited to, phosphors and/or fluorophores, which afford phosphorescence and/or fluorescence, respectively. In some embodiments, the luminescent compound can fluoresce (e.g., is a fluorescent compound). In some embodiments, a compound of the present invention includes a first porphyrin and a first hydroporphyrin, wherein the first porphyrin is attached to the first hydroporphyrin. In some embodiments, the first porphyrin is attached to the first hydroporphyrin via a linking group. In some embodiments, the first porphyrin is attached to the first hydroporphyrin via a direct bond. In some embodiments, two or more porphyrins are linked (either directly or via a linking group) to a hydroporphyrin (e.g., a first hydroporphyrin). A compound of the present invention includes at least one porphyrin that is a donor and at least one hydroporphyrin that is an acceptor. It was unexpectedly discovered that a compound of the present invention can provide a donor-acceptor (e.g., porphyrin-hydroporphyrin) energy transfer and, in some embodiments, a higher fluorescence quantum yield and/or simpler (e.g., less complex) spectra than would be expected for the fluorescence quantum yield and/or spectra based on the spectral properties of the porphyrin alone. Typically, a porphyrin would not be expected to be a suitable donor since porphyrins can have low fluorescence quantum yields and complex emission spectra (e.g., two or more emission peaks).

In some embodiments of the invention, a compound of the invention comprises a porphyrin having a structure of one of Formula Ia or Formula Ib:

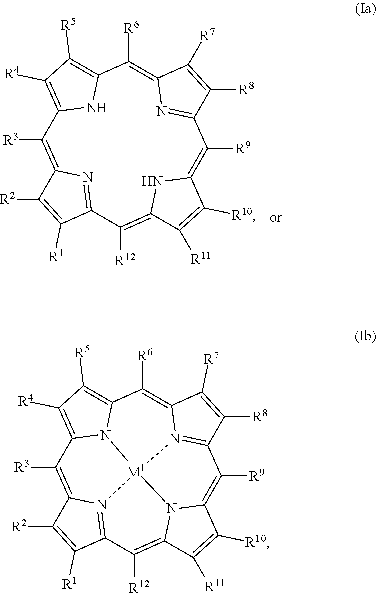

wherein:
R¹, R², R³, R⁴, R⁵, R⁶, R⁷, R⁸, R⁹, R¹⁰, R¹¹, and R¹² are each independently selected from the group consisting of a hydrogen, alkyl, alkenyl, alkynyl, cycloalkyl, cycloalkylalkyl, cycloalkylalkenyl, cycloalkylalkynyl, heterocyclo, heterocycloalkyl, heterocycloalkenyl, heterocycloalkynyl, aryl, aryloxy, arylalkyl, arylalkenyl, arylalkynyl, heteroaryl, heteroarylalkyl, heteroarylalkenyl, heteroarylalkynyl, alkoxy, halo, mercapto, azido, cyano, formyl, carboxylic acid, hydroxyl, nitro, acyl, alkylthio, amino, alkylamino, arylalkylamino, disubstituted amino, acylamino, acyloxy, ester, amide, sulfoxyl, sulfonyl, sulfonate, sulfonic acid, sulfonamide, urea, alkoxylacylamino, aminoacyloxy, hydrophilic groups, linking groups, bioconjugatable groups, surface attachment groups, and targeting groups;
or R¹ and R² together represent a fused aromatic or heteroaromatic ring system that is substituted or unsubstituted;
or R³ and R⁵ together represent a fused aromatic or heteroaromatic ring system that is substituted or unsubstituted;
   or R⁴ and R⁵ together represent a fused aromatic or heteroaromatic ring system that is substituted or unsubstituted;
   or R⁴ and R⁷ together represent a fused aromatic or heteroaromatic ring system that is substituted or unsubstituted;
or R⁷ and R⁸ together represent a fused aromatic or heteroaromatic ring system that is substituted or unsubstituted;
or R⁹ and R¹⁰ together represent a fused aromatic or heteroaromatic ring system that is substituted or unsubstituted; or
or R¹⁰ and R¹¹ together represent a fused aromatic or heteroaromatic ring system that is substituted or unsubstituted; and
M¹, if present, is a metal (e.g., zinc, magnesium, gold, aluminum, silicon, palladium, indium, tin, copper, or platinum), and
wherein at least one of R¹, R², R³, R⁴, R⁵, R⁶, R⁷, R⁸, R⁹, R¹⁰, R¹¹, and R¹² is an attachment point to a hydroporphyrin or a porphyrin via a linking group or a direct bond to the hydroporphyrin or porphyrin. A fused aromatic or heteroaromatic ring system of a compound of Formula Ia or Formula Ib may be substituted with one or more substituents such as, but not limited to, a substituent selected from the group consisting of halo, alkyl, haloalkyl, alkenyl, alkynyl, cycloalkyl, cycloalkylalkyl, aryl, arylalkyl, heterocyclo, heterocycloalkyl, hydroxyl, alkoxy (thereby creating a polyalkoxy such as polyethylene glycol), alkenyloxy, alkynyloxy, haloalkoxy, cycloalkoxy, cycloalkylalkyloxy, aryloxy, arylalkyloxy, heterocyclooxy, heterocycloalkyloxy, mercapto, alkyl-S(O)m, haloalkyl-S(O)m, alkenyl-S(O)m, alkynyl-S(O)m, cycloalkyl-S(O)m, cycloalkylalkyl-S(O)m, aryl-S(O)m, arylalkyl-S(O)m, heterocyclo-S(O)m, heterocycloalkyl-S(O)m, amino, carboxy, alkylamino, alkenylamino, alkynylamino, haloalkylamino, cycloalkylamino, cycloalkylalkylamino, arylamino, arylalkylamino, heterocycloamino, heterocycloalkylamino, disubstituted-amino, acylamino, acyloxy, ester, amide, sulfonamide, urea, alkoxyacylamino, aminoacyloxy, nitro, and cyano where m=0, 1, 2 or 3. In some embodiments, a fused aromatic or heteroaromatic ring system of a compound of Formula Ia or Formula Ib may be substituted with an ester or amine.

In some embodiments, one of R¹, R², R³, R⁴, R⁵, R⁶, R⁷, R⁸, R⁹, R¹⁰, R¹¹, and R¹² of Formula Ia or Formula Ib is bound to a hydroporphyrin via a direct bond. In some embodiments, one of R¹, R², R³, R⁴, R⁵, R⁶, R⁷, R⁸, R⁹, R¹⁰, R¹¹, and R¹² of Formula Ia or Formula Ib is bound to a linking group that is bound to a hydroporphyrin. In some embodiments, a first porphyrin having a structure of Formula Ia or Formula Ib is bound to a second porphyrin via a direct bond at one of R¹, R², R³, R⁴, R⁵, R⁶, R⁷, R⁸, R⁹, R¹⁰, R¹¹, and R¹² of Formula Ia or Formula Ib to the second porphyrin that optionally has the same or a different structure than the first porphryin. In some embodiments, a first porphyrin having a structure of Formula Ia or Formula Ib is bound to a linking group at one of R¹, R², R³, R⁴, R⁵, R⁶, R⁷, R⁸, R⁹, R¹⁰, R¹¹, and R¹² of Formula Ia or Formula Ib and the linking group is attached to a second porphyrin that optionally has the same or a different structure than the first porphryin. In some embodiments, one or more of R³, R⁶, R⁹, and R¹² of Formula Ia or Formula Ib is independently bound to a hydroporphyrin or a porphyrin via a linking group or a direct bond to the hydroporphyrin or porphyrin. In some embodiments, R⁶ of Formula Ia or Formula Ib is bound to a hydroporphyrin or a porphyrin via a linking group or a direct bond to the hydroporphyrin or porphyrin.

In some embodiments, a compound of the present invention comprises a porphyrin having a structure of Formula Ia. The porphyrin of Formula Ia is devoid of a metal ion in the center of the porphyrin (e.g., devoid of a metal ion in the cavity/core of the porphyrin) and is thus in the free base form. A porphyrin of Formula Ia may also be referred to herein as a free base porphyrin. In some embodiments, a compound of the present invention comprises a porphyrin having a structure of Formula Ib and M¹ is a metal that is optionally zinc, magnesium, gold, aluminum, silicon, palladium, indium, tin, copper, or platinum. In some embodiments, a compound of the present invention comprises a porphyrin having a structure of Formula Ib and M¹ is zinc. In some embodiments, a compound of the present invention comprises a porphyrin having a structure of Formula Ib and M¹ is magnesium.

In some embodiments of the invention, R¹, R², R³, R⁴, R⁵, R⁶, R⁷, R⁸, R⁹, R¹⁰, R¹¹, and R¹² of Formula Ia or Formula Ib are each independently selected from the group consisting of hydrogen, alkyl, alkenyl, alkynyl, cycloalkyl, cycloalkylalkyl, cycloalkylalkenyl, cycloalkylalkynyl, heterocyclo, heterocycloalkyl, heterocycloalkenyl, heterocycloalkynyl, aryl, aryloxy, arylalkyl, arylalkenyl, arylalkynyl, heteroaryl, heteroarylalkyl, heteroarylalkenyl, heteroarylalkynyl, alkoxy, halo, mercapto, azido, cyano, formyl, carboxylic acid, hydroxyl, nitro, acyl, alkylthio, amino, alkylamino, arylalkylamino, disubstituted amino, acylamino, acyloxy, ester, amide, sulfoxyl, sulfonyl, sulfonate, sulfonic acid, sulfonamide, urea, alkoxylacylamino, aminoacyloxy, hydrophilic groups, linking groups, bioconjugatable groups, surface attachment groups, and targeting groups; and M¹, if present, is a metal (e.g., zinc, magnesium, gold, aluminum, silicon, palladium, indium, tin, copper, or platinum), and wherein at least one of R¹, R², R³, R⁴, R⁵, R⁶, R⁷, R⁸, R⁹, R¹⁰, R¹¹, and R¹² is bound to a hydroporphyrin or a porphyrin via a linking group or a direct bond to the hydroporphyrin or porphyrin

In particular embodiments of the invention, R¹, R², R³, R⁴, R⁵, R⁶, R⁷, R⁸, R⁹, R¹⁰, R¹¹, and R¹² of Formula Ia or Formula Ib are each independently selected from the group consisting of hydrogen, halo, carboxy, cyano, carboxylic acid, alkyl, alkenyl, alkynl, acyl, acyloxy, sulfonyl, sulfoxyl, amino, amido, nitro, hydroxy, mercapto, alkoxy, ester, phenyl, substituted phenyl, surface attachment groups, linking groups, bioconjugatable groups, targeting groups, hydrophilic groups, and combinations thereof, and wherein at least one of R¹, R², R³, R⁴, R⁵, R⁶, R⁷, R⁸, R⁹, R¹⁰, R¹¹, and R¹² is bound to a hydroporphyrin or a porphyrin via a linking group or a direct bond to the hydroporphyrin or porphyrin, optionally wherein one of R³, R⁶, R⁹, and R¹² is bound to a hydroporphyrin or a porphyrin via a linking group or a direct bond to the hydroporphyrin or porphyrin. In some embodiments, at least one of R³, R⁶, R⁹, and R¹² is a halo, carboxy, cyano, carboxylic acid, alkyl, alkenyl, alkynl, acyl, acyloxy, sulfonyl, sulfoxyl, amino, amido, nitro, hydroxy, mercapto, alkoxy, ester, phenyl, substituted phenyl, a surface attachment group, a linking group, a bioconjugatable group, a targeting group, or a hydrophilic group, and one of R³, R⁶, R⁹, and R¹² is bound to a hydroporphyrin or a porphyrin via a linking group or a direct bond to the hydroporphyrin or porphyrin.

In some embodiments of the invention, R¹, R², R³, R⁴, R⁵, R⁶, R⁷, R⁸, R⁹, R¹⁰, R¹¹, and R¹² of Formula Ia or Formula Ib are each independently selected from the group consisting of hydrogen, alkylester (e.g., methyl ester, ethyl ester), alkylbenzoate, (e.g., 4-methylbenzoate, 4-ethylbenzoate), phenyl (e.g., a substituted or unsubstituted phenyl), carboxy(alkyl or ester)alkyl phenyl (e.g., 3-(4-carboxybutyl)phenyl), trimethylphenyl (e.g., mesityl), and combinations thereof, optionally wherein one of R³, R⁶, R⁹, and R¹² is bound to a hydroporphyrin or a porphyrin via a linking group or a direct bond to the hydroporphyrin or porphyrin. In some embodiments, at least one of R³, R⁶, R⁹, and R¹² is an alkylester (e.g., methyl ester, ethyl ester), alkylbenzoate, (e.g., 4-methylbenzoate, 4-ethylbenzoate), phenyl (e.g., a substituted or unsubstituted phenyl), carboxy(alkyl or ester)alkyl phenyl (e.g., 3-(4-carboxybutyl)phenyl), or trimethylphenyl (e.g., mesityl), and one of R³, R⁶, R⁹, and R¹² is bound to a hydroporphyrin or a porphyrin via a linking group or a direct bond to the hydroporphyrin or porphyrin.

In some embodiments, a compound of the present invention comprises a porphyrin having a structure of Formula Ia or Tb and the porphyrin is excited at 405 nm. In some embodiments, a compound of the present invention comprises a porphyrin having a structure of Formula Ia or Tb and the porphyrin is excited at 488 nm. In some embodiments, a compound of the present invention comprises a porphyrin having a structure of Formula Ia or Tb and the porphyrin is excited at 407 nm.

In some embodiments of the invention, the compound of Formula Ia or Tb includes two or less aryl or heteroaryl substituents. In some embodiments, the porphyrin of Formula Ta or Formula Ib has 1 or 2 aryl substituents. In some embodiments, the porphyrin of Formula Ta or Formula Ib has 1 or 2 heteroaryl substituents. In some embodiments, the compound of Formula Ia or Tb includes two or more aryl or heteroaryl substituents. For example, an exemplary porphyrin (such as a porphyrin that can be excited at about 488 nm) may have a structure of Formula Ib″:

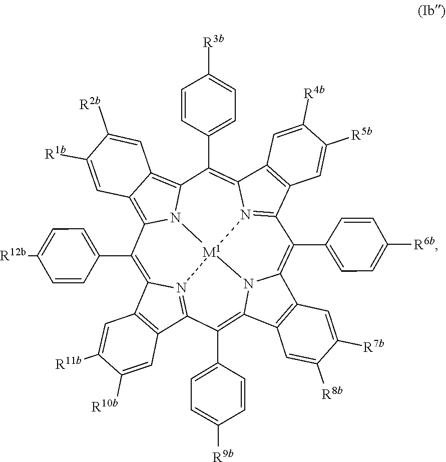

wherein M¹ is a metal (e.g., zinc, magnesium, gold, aluminum, silicon, palladium, indium, tin, copper, or platinum), and R^{1b}, R^{2b}, R^{3b}, R^{4b}, R^{5b}, R^{6b}, R^{7b}, R^{8b}, R^{9b}, R^{10b}, R^{11b}, and R^{12b} of Formula Ib″ are each independently a hydrogen, a substituent (e.g., an alkyl, ester, or amine), or a direct bond to a hydroporphyrin or a porphyrin. In some embodiments, R^{1b}, R^{2b}R^{4b}, R^{5b}, R^{7b}, R^{8b}, R^{10b}, and R^{11b} of Formula Ib″ are each independently a hydrogen, ester, or amine, and R^{3b}, R^{6b}, R^{9b}, and R^{12b} of Formula Ib″ are each independently a hydrogen or a direct bond to a hydroporphyrin or a porphyrin, wherein one or more of R^{3b}, R^{6b}, R^{9b}, and R^{12b} of Formula Ib″ is/are a direct bond to a hydroporphyrin or a porphyrin. In some embodiments, a porphyrin of Formula Ib″ is excited at about 488 nm.

In some embodiments, a porphyrin (such as a porphyrin that can be excited at about 445 nm) may have a structure of Formula Ia′″:

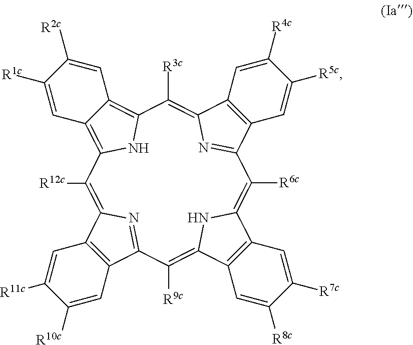

wherein R^{1c}, R^{2c}, R^{3c}, R^{4c}, R^{5c}, R^{6c}, R^{7c}, R^{8c}, R^{9c}, R^{10c}, R^{11c}, and R^{12c} of Formula Ia′″ are each independently a hydrogen, a substituent (e.g., an alkyl, ester, or amine), or a direct bond to a hydroporphyrin or a porphyrin. In some embodiments, one or more of R^{1c}, R^{2c}, R^{3c}, R^{4c}, R^{5c}, R^{7c}, R^{8c}, R^{10c}, and R^{11c} of Formula Ia′″ are each independently a hydrogen or an ester. In some embodiments, one or more of R^{1c}, R^{2c}, R^{3c}, R^{4c}, R^{5c}, R^{7c}, R^{8c}, R^{10c}, and R^{11c} of Formula Ia′″ is an ester, optionally an alkyl ester. In some embodiments, one or more of R^{1c}, R^{2c}, R^{3c}, R^{4c}, R^{5c}, R^{7c}, R^{8c}, R^{10c}, and R^{11c} of Formula Ia′″ is —CO₂(alkyl), optionally wherein the alkyl is methyl, ethyl, propyl, or butyl (e.g., n-butyl, sec-butyl, isobutyl, or tert-butyl). In some embodiments, R^{1c}, R^{2c}, R^{3c}, R^{4c}, R^{5c}, R^{7c}, R^{8c}, R^{11c}, and R^{11c} of Formula Ia′″ are each independently —CO₂CH₃ or —CO₂(CH₂)₃CH₃. In some embodiments, at least one of R^{3c}, R^{6c}, R^{9c}, and R^{12c} of Formula Ia′″ is bound to a hydroporphyrin via a linking group (e.g., a linking group comprising a phenyl) or is a direct bond to a hydroporphyrin. In some embodiments, a porphyrin of Formula Ia′″ is excited at about 445 nm. In some embodiments, a porphyrin of Formula Ib′″ is excited at about 447 nm.

In some embodiments, a compound of the present invention comprises a hydroporphyrin and the hydroporphyrin is a chlorin. In some embodiments, a compound of the present invention comprises a hydroporphyrin and the hydrophorphyrin is a bacteriochlorin. In some embodiments, the bacteriochlorin is an isobacteriochlorin or an azabacteriochlorin.

In some embodiments of the invention, a compound of the invention comprises a hydroporphyrin having a structure of one of Formula IIa-IId:

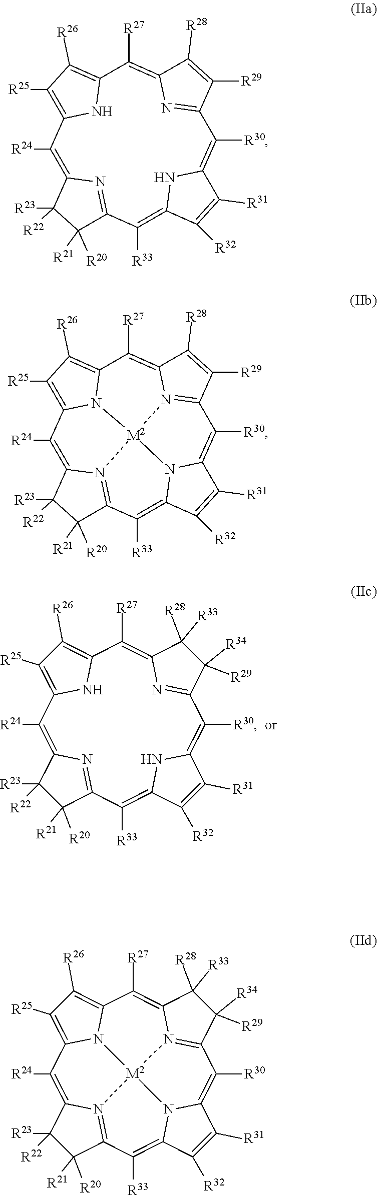

wherein:
R²⁰, R²¹, R²², R²³, R²⁴, R²⁵, R²⁶, R²⁷, R²⁸, R²⁹, R³⁰, R³¹, R³², R³³, and R³⁴ are each independently selected from the group consisting of hydrogen, alkyl, alkenyl, alkynyl, cycloalkyl, cycloalkylalkyl, cycloalkylalkenyl, cycloalkylalkynyl, heterocyclo, heterocycloalkyl, heterocycloalkenyl, heterocycloalkynyl, aryl, aryloxy, arylalkyl, arylalkenyl, arylalkynyl, heteroaryl, heteroarylalkyl, heteroarylalkenyl, heteroarylalkynyl, alkoxy, halo, mercapto, azido, cyano, formyl, carboxylic acid, hydroxyl, nitro, acyl, alkylthio, amino, alkylamino, arylalkylamino, disubstituted amino, acylamino, acyloxy, ester, amide, sulfoxyl, sulfonyl, sulfonate, sulfonic acid, sulfonamide, urea, alkoxylacylamino, aminoacyloxy, hydrophilic groups, linking groups, bioconjugatable groups, surface attachment groups, and targeting groups;
or R²⁰ and R²¹ together are ═O or spiroalkyl;
or R²² and R²³ together are ═O or spiroalkyl;
or R²⁸ and R³³ together are ═O or spiroalkyl;
or R²⁹ and R³³ together are ═O or spiroalkyl;
or R²⁴ and R²⁵ together represent a fused aromatic or heteroaromatic ring system that is substituted or unsubstituted;
or R²⁵ and R²⁶ together represent a fused aromatic or heteroaromatic ring system that is substituted or unsubstituted;
or R²⁶ and R²⁷ together represent a fused aromatic or heteroaromatic ring system that is substituted or unsubstituted;
or R³⁰ and R³¹ together represent a fused aromatic or heteroaromatic ring system that is substituted or unsubstituted;
or R³¹ and R³² together represent a fused aromatic or heteroaromatic ring system that is substituted or unsubstituted; or
or R³² and R³³ together represent a fused aromatic or heteroaromatic ring system that is substituted or unsubstituted; and
M², if present, is a metal (e.g., zinc, magnesium, gold, aluminum, silicon, palladium, indium, tin, copper, or platinum), and
wherein at least one of R²⁰, R²¹, R²², R²³, R²⁴, R²⁵, R²⁶, R²⁷, R²⁸, R²⁹, R³⁰, R³¹, R³², R³³, and R³⁴ is an attachment point to a hydroporphyrin or a porphyrin via a linking group or a direct bond to the hydroporphyrin or porphyrin. A fused aromatic or heteroaromatic ring system of a compound of Formula IIa-IId may be substituted with one or more substituents such as, but not limited to, a substituent selected from the group consisting of halo, alkyl, haloalkyl, alkenyl, alkynyl, cycloalkyl, cycloalkylalkyl, aryl, arylalkyl, heterocyclo, heterocycloalkyl, hydroxyl, alkoxy (thereby creating a polyalkoxy such as polyethylene glycol), alkenyloxy, alkynyloxy, haloalkoxy, cycloalkoxy, cycloalkylalkyloxy, aryloxy, arylalkyloxy, heterocyclooxy, heterocycloalkyloxy, mercapto, alkyl-S(O)m, haloalkyl-S(O)m, alkenyl-S(O)m, alkynyl-S(O)m, cycloalkyl-S(O)m, cycloalkylalkyl-S(O)m, aryl-S(O)m, arylalkyl-S(O)m, heterocyclo-S(O)m, heterocycloalkyl-S(O)m, amino, carboxy, alkylamino, alkenylamino, alkynylamino, haloalkylamino, cycloalkylamino, cycloalkylalkylamino, arylamino, arylalkylamino, heterocycloamino, heterocycloalkylamino, disubstituted-amino, acylamino, acyloxy, ester, amide, sulfonamide, urea, alkoxyacylamino, aminoacyloxy, nitro, and cyano where m=0, 1, 2 or 3.

In some embodiments, one of R²⁰, R²¹, R²², R²³, R²⁴, R²⁵, R²⁶, R²⁷, R²⁸, R²⁹, R³⁰, R³¹, R³², R³³, and R³⁴ of Formula IIa-IId is bound to a porphyrin via a direct bond. In some embodiments, one of R²⁰, R²¹, R²², R²³, R²⁴, R²⁵, R²⁶, R²⁷, R²⁸, R²⁹, R³⁰, R³¹, R³², R³³, and R³⁴ of Formula IIa-IId is bound to a linking group that is bound to a porphyrin. In some embodiments, a first hydroporphyrin having a structure of Formula IIa-IId is bound to a second hydroporphyrin via a direct bond at one of R²⁰, R²¹, R²², R²³, R²⁴, R²⁵, R²⁶, R²⁷, R²⁸, R²⁹, R³⁰, R³¹, R³², R³³, and R³⁴ of Formula IIa-IId to the second hydroporphyrin that optionally has the same or a different structure than the first hydroporphyrin. In some embodiments, a first hydroporphyrin having a structure of Formula IIa-IId is bound to a linking group at one of R²⁰, R²¹, R²², R²³, R²⁴, R²⁵, R²⁶, R²⁷, R²⁸, R²⁹, R³⁰, R³¹, R³², R³³, and R³⁴ of Formula Ia or Formula Ib and the linking group is attached to a second hydroporphyrin that optionally has the same or a different structure than the first hydroporphyrin.

In some embodiments, at least one of R²⁴, R²⁵, R²⁶, R²⁷, R³⁰, R³¹, R³², and R³³ of Formula IIa-IId is independently bound to a hydroporphyrin or a porphyrin via a linking group or a direct bond to the hydroporphyrin or porphyrin. In some embodiments, two or more (e.g., 2, 3, 4, 5, 6, 7, or 8) of R²⁴, R²⁵, R²⁶, R²⁷, R³⁰, R³¹, R³², and R³³ of Formula IIa-IId is independently bound to a hydroporphyrin or a porphyrin via a linking group or a direct bond to the hydroporphyrin or porphyrin. In some embodiments, one of R²⁴, R²⁷, R³⁰, and R³³ of Formula IIa-IId is bound to a hydroporphyrin or a porphyrin via a linking group or a direct bond to the hydroporphyrin or porphyrin. In some embodiments, one of R²⁵ and R²⁶ of Formula IIa-IId is bound to a hydroporphyrin or a porphyrin via a linking group or a direct bond to the hydroporphyrin or porphyrin. In some embodiments, one of R³¹ and R³² of Formula IIa-IId is bound to a hydroporphyrin or a porphyrin via a linking group or a direct bond to the hydroporphyrin or porphyrin. In some embodiments, R³⁰ of Formula IIa-IId is bound to a hydroporphyrin or a porphyrin via a linking group or a direct bond to the hydroporphyrin or porphyrin, optionally wherein the compound has a structure of Formula IIa or IIb. In some embodiments, R³³ of Formula IIa-IId is bound to a hydroporphyrin or a porphyrin via a linking group or a direct bond to the hydroporphyrin or porphyrin, optionally wherein the compound has a structure of Formula IIc or IId. In some embodiments, R³² of Formula IIa-IId is bound to a hydroporphyrin or a porphyrin via a linking group or a direct bond to the hydroporphyrin or porphyrin. In some embodiments, R²⁶ of Formula IIa-IId is bound to a hydroporphyrin or a porphyrin via a linking group or a direct bond to the hydroporphyrin or porphyrin. In some embodiments, R²⁵ and R³¹ of Formula IIa-IId are each independently bound to a hydroporphyrin or a porphyrin via a linking group or a direct bond to the hydroporphyrin or porphyrin. In some embodiments, R²⁶ and R³² of Formula IIa-IId are each independently bound to a hydroporphyrin or a porphyrin via a linking group or a direct bond to the hydroporphyrin or porphyrin.

In some embodiments, a compound of the present invention comprises a hydroporphyrin having a structure of Formula IIa-IId that is bound to a porphyrin having a structure of Formula Ia or Formula Ib via a direct bond at R²⁰, R²¹, R²², R²³, R²⁴, R²⁵, R²⁶, R²⁷, R²⁸, R²⁹, R³⁰, R³¹, R³², R³³, or R³⁴ of Formula IIa-IId to R¹, R², R³, R⁴, R⁵, R⁶, R⁷, R⁸, R⁹, R¹⁰, R¹¹, or R¹² of Formula Ia or Formula Ib. In some embodiments, a compound of the present invention comprises a hydroporphyrin having a structure of Formula IIa-IId that is bound to a porphyrin having a structure of Formula Ia or Formula Ib via a linking group that is bound to R²⁰, R²¹, R²², R²³, R²⁴, R²⁵, R²⁶, R²⁷, R²⁸, R²⁹, R³⁰, R³¹, R³², R³³, or R³⁴ of Formula IIa-IId and the linking group is bound to R¹, R², R³, R⁴, R⁵, R⁶, R⁷, R⁸, R⁹, R¹⁰, R¹¹, or R¹² of Formula Ia or Formula Ib. In some embodiments, a compound of the present invention comprises a hydroporphyrin having a structure of Formula IIa-IId that is bound to a porphyrin having a structure of Formula Ia or Formula Ib via a linking group that is bound to R²⁴, R²⁵, R²⁶, R²⁷, R³⁰, R³³, R³¹ or R³² of Formula IIa-IId and the linking group is bound to R³, R⁶, R⁹, or R¹² of Formula Ia or Formula Ib.

In some embodiments, a compound of the present invention comprises a hydroporphyrin having a structure of Formula IIa. In some embodiments, a compound of the present invention comprises a hydroporphyrin having a structure of Formula IIb and M¹ is a metal that is optionally zinc, magnesium, gold, aluminum, silicon, palladium, indium, tin, copper, or platinum. In some embodiments, a compound of the present invention comprises a hydroporphyrin having a structure of Formula IIc. In some embodiments, a compound of the present invention comprises a hydroporphyrin having a structure of Formula IId and M¹ is a metal that is optionally zinc, magnesium, gold, aluminum, silicon, palladium, indium, tin, copper, or platinum. In some embodiments, a compound of the present invention comprises a hydroporphyrin having a structure of Formula IIb or Formula IId and M¹ is zinc. In some embodiments, a compound of the present invention comprises a hydroporphyrin having a structure of Formula IIb or Formula IId and M¹ is magnesium. The hydroporphyrin of Formula IIa and the hydroporphyrin of Formula IIc are each devoid of a metal ion in the center of the hydroporphyrin (e.g., devoid of a metal ion in the cavity/core of the hydroporphyrin) and thus each is in the free base form. A hydroporphyrin of Formula IIa and/or Formula IIc may also be referred to herein as a free base hydroporphyrin.

In some embodiments of the invention, R²⁰, R²¹, R²², R²³, R²⁴, R²⁵, R²⁶, R²⁷, R²⁸, R²⁹, R³⁰, R³¹, R³², R³³, and R³⁴ of Formula IIa-IId are each independently selected from the group consisting of hydrogen, alkyl, alkenyl, alkynyl, cycloalkyl, cycloalkylalkyl, cycloalkylalkenyl, cycloalkylalkynyl, heterocyclo, heterocycloalkyl, heterocycloalkenyl, heterocycloalkynyl, aryl, aryloxy, arylalkyl, arylalkenyl, arylalkynyl, heteroaryl, heteroarylalkyl, heteroarylalkenyl, heteroarylalkynyl, alkoxy, halo, mercapto, azido, cyano, formyl, carboxylic acid, hydroxyl, nitro, acyl, alkylthio, amino, alkylamino, arylalkylamino, disubstituted amino, acylamino, acyloxy, ester, amide, sulfoxyl, sulfonyl, sulfonate, sulfonic acid, sulfonamide, urea, alkoxylacylamino, aminoacyloxy, hydrophilic groups, linking groups, bioconjugatable groups, surface attachment groups, and targeting groups, and wherein at least one of R²⁰, R²¹, R²², R²³, R²⁴, R²⁵, R²⁶, R²⁷, R²⁸, R²⁹, R³⁰, R³¹, R³², R³³, and R³⁴ is bound to a hydroporphyrin or a porphyrin via a linking group or a direct bond to the hydroporphyrin or porphyrin.

In some embodiments of the invention, R²⁰, R²¹, R²², R²³, R²⁴, R²⁵, R²⁶, R²⁷, R²⁸, R²⁹, R³⁰, R³¹, R³², R³³, and R³⁴ of Formula IIa-IId are each independently selected from the group consisting of hydrogen, alkyl, alkenyl, alkynyl, cycloalkyl, cycloalkylalkyl, cycloalkylalkenyl, cycloalkylalkynyl, heterocyclo, heterocycloalkyl, heterocycloalkenyl, heterocycloalkynyl, aryl, aryloxy, arylalkyl, arylalkenyl, arylalkynyl, heteroaryl, heteroarylalkyl, heteroarylalkenyl, heteroarylalkynyl, alkoxy, halo, mercapto, azido, cyano, formyl, carboxylic acid, hydroxyl, nitro, acyl, alkylthio, amino, alkylamino, arylalkylamino, disubstituted amino, acylamino, acyloxy, ester, amide, sulfoxyl, sulfonyl, sulfonate, sulfonic acid, sulfonamide, urea, alkoxylacylamino, aminoacyloxy, hydrophilic groups, linking groups, bioconjugatable group, surface attachment groups, and targeting groups, and wherein at least one of R²⁰, R²¹, R²², R²³, R²⁴, R²⁵, R²⁶, R²⁷, R²⁸, R²⁹, R³⁰, R³¹, R³², R³³, and R³⁴ is bound to a hydroporphyrin or a porphyrin via a linking group or a direct bond to the hydroporphyrin or porphyrin.

In some embodiments of the invention, R²⁰, R²¹, R²², R²³, R²⁴, R²⁵, R²⁶, R²⁷, R²⁸, R²⁹, R³⁰, R³¹, R³², R³³, and R³⁴ of Formula IIa-IId are each independently selected from the group consisting of hydrogen, halo, carboxy, cyano, carboxylic acid, alkyl, alkenyl, alkynl, acyl, acyloxy, sulfonyl, sulfoxyl, amino, amido, nitro, hydroxy, mercapto, alkoxy, ester, phenyl, substituted phenyl, surface attachment groups, linking groups, bioconjugatable groups, targeting groups, hydrophilic groups, and combinations thereof, and wherein at least one of R²⁰, R²¹, R²², R²³, R²⁴, R²⁵, R²⁶, R²⁷, R²⁸, R²⁹, R³⁰, R³¹, R³², R³³, and R³⁴ is bound to a hydroporphyrin or a porphyrin via a linking group or a direct bond to the hydroporphyrin or porphyrin. In some embodiments, at least one of R²⁴, R²⁵, R²⁶, R²⁷, R³⁰, and R³³ is a halo, carboxy, cyano, carboxylic acid, alkyl, alkenyl, alkynl, acyl, acyloxy, sulfonyl, sulfoxyl, amino, amido, nitro, hydroxy, mercapto, alkoxy, ester, phenyl, substituted phenyl, surface attachment group, linking group, targeting group, or hydrophilic group; and one of R²⁴, R²⁵, R²⁶, R²⁷, R³⁰, R³³, R³¹ and R³² is bound to a hydroporphyrin or a porphyrin via a linking group or a direct bond to the hydroporphyrin or porphyrin.

In some embodiments of the invention, R²⁰, R²¹, R²², R²³, R²⁴, R²⁵, R²⁶, R²⁷, R²⁸, R²⁹, R³⁰, R³¹, R³², R³³, and R³⁴ of Formula IIa-IId are each independently hydrogen, phenyl, halophenyl, alkoxy (e.g., methoxy, ethoxy), alkylester (e.g., methyl ester, ethyl ester), alkylbenzoate, (e.g., 4-methylbenzoate, 4-ethylbenzoate), carboxy(alkyl or ester)alkyl phenyl (e.g., 3-(4-carboxybutyl)phenyl), trimethylphenyl (e.g., mesityl), alkylcarboxylic acid, alkylalkylester, (alkylester)phenylethynyl, a linking group, a bioconjugatable group, a surface attachment group, a targeting group, and combinations thereof, and wherein at least one of R²⁰, R²¹, R²², R²³, R²⁴, R²⁵, R²⁶, R²⁷, R²⁸, R²⁹, R³⁰, R³¹, R³², R³³, and R³⁴ is bound to a hydroporphyrin or a porphyrin via a linking group or a direct bond to the hydroporphyrin or porphyrin. In some embodiments, at least one of R²⁴, R²⁵, R²⁶, R²⁷, R³⁰, and R³³ is a phenyl, halophenyl, alkoxy (e.g., methoxy, ethoxy), alkylester (e.g., methyl ester, ethyl ester), alkylbenzoate, (e.g., 4-methylbenzoate, 4-ethylbenzoate), carboxy(alkyl or ester)alkyl phenyl (e.g., 3-(4-carboxybutyl)phenyl), trimethylphenyl (e.g., mesityl), alkylcarboxylic acid, alkylalkylester, (alkylester)phenylethynyl, a linking group, a bioconjugatable group, a surface attachment group, or a targeting group; and one of R²⁴, R²⁵, R²⁶, R²⁷, R³⁰, R³³, R³¹, and R³² is bound to a hydroporphyrin or a porphyrin via a linking group or a direct bond to the hydroporphyrin or porphyrin.

In some embodiments, at least one of R²⁰, R²¹, R²², R²³, R²⁴, R²⁵, R²⁶, R²⁷, R²⁸, R²⁹, R³⁰, R³¹, R³², R³³, and R³⁴ of Formula IIa-IId has a structure of Formula A or Formula B

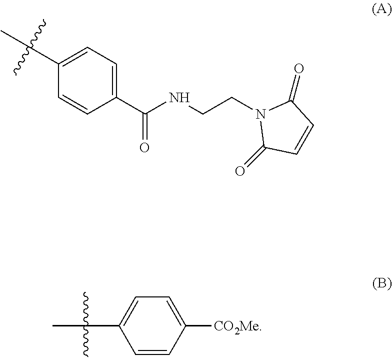 [SMILES: *c1ccc(C(=O)NCCN2C(=O)C=CC2=O)cc1 ; *c1ccc(C(=O)OC)cc1]

In some embodiments, in the compound of Formula IIa-IId, at least one of R²⁴, R²¹, R²⁶, R²⁷, R³⁰, and R³³ has a structure of Formula A or a structure of Formula B, and one of R²⁴, R²⁷, R³⁰, R³³, R³¹, and R³² is bound to a hydroporphyrin or a porphyrin via a linking group or a direct bond to the hydroporphyrin or porphyrin.

In some embodiments, a compound of the present invention comprises a porphyrin that is bound to a hydroporphyrin via a direct bond or a linking group at a meso position of the porphyrin and at a beta position of the hydroporphyrin. As such, in particular embodiments, a compound of the present invention may comprise a hydroporphyrin that is attached to a porphyrin having a structure of Formula Ia or Formula Ib, wherein the hydroporphyrin (optionally at a beta position of the hydroporphyrin) is attached (via a direct bond or linking group) to R³, R⁶, R⁹, or R¹² of Formula Ia or Formula Ib. In some embodiments, a compound of the present invention comprises a porphyrin that is bound to a hydroporphyrin via a direct bond or a linking group at a meso position of the porphyrin and at a meso position of the hydroporphyrin. In some embodiments, a compound of the present invention comprises a porphyrin that is bound to a hydroporphyrin via a direct bond or a linking group at a beta position of the porphyrin and at a meso position of the hydroporphyrin. In some embodiments of the invention, a compound of the present invention comprises a hydroporphyrin that is bound to a porphyrin via a direct bond or a linking group at a beta position of the hydroporphyrin and at a beta position of the porphyrin.

In some embodiments of the invention, a compound of the present invention comprises a first hydroporphyrin that has a structure of Formula IIa or IIb, and R³² is a direct bond to a first porphyrin (e.g., having a structure of Formula Ia or Formula Ib) or a bond to a linking group that is bonded to the first porphyrin; and R³⁰ is a bioconjugatable group, such as a carboxylic acid or ester thereof, amine, isothiocyanate, isocyanate, maleimide, and iodoacetamide. In some embodiments, R³⁰ of Formula IIa or IIb has a structure of Formula A or Formula B.

In some embodiments of the invention, a compound of the present invention comprises a first hydroporphyrin that has a structure of Formula IIc or IId, and R³² is a direct bond to a first porphyrin (e.g., having a structure of Formula Ia or Formula Ib) or a bond to a linking group that is bonded to the first porphyrin; and R³⁰ is a bioconjugatable group, such as a carboxylic acid or ester thereof, amine, isothiocyanate, isocyanate, maleimide, and iodoacetamide. In some embodiments, R³⁰ of Formula IIc or IId has a structure of Formula A or Formula B.

In some embodiments of the invention, a compound of the present invention comprises a first hydroporphyrin that has a structure of Formula IIa or Formula IIb and R²⁰, R²¹, R²², and R²³ are each independently hydrogen or alkyl (e.g., methyl). In some embodiments, one, two, three, or all of R²⁰, R²¹, R²², and R²³ of Formula IIa or Formula IIb is/are an alkyl (e.g., methyl). In some embodiments, a compound of the present invention comprises a first hydroporphyrin that has a structure Formula IIc or Formula IId and R²⁰, R²¹, R²², R²³, R²⁸, R²⁹, R³³, and R³⁴ are each independently hydrogen or alkyl (e.g., methyl). In some embodiments, one, two, three, four, five, six, seven, or all of R²⁰, R²¹, R²², R²³, R²⁸, R²⁹, R³³, and R³⁴ of Formula IIc or Formula IId is/are an alkyl (e.g., methyl). In some embodiments, a compound of the present invention comprises a first hydroporphyrin (e.g., a hydroporphyrin that has a structure of one of Formula IIa-IId) having a geminal dialkyl group in each reduced, pyrroline ring, optionally wherein the hydroporphyrin comprises a geminal dimethyl group.

In some embodiments, a compound of the present invention includes one or more porphyrin(s) (e.g., 1, 2, 3, 4, or more porphyrins). In some embodiments, a compound of the invention includes a first porphyrin, a second porphyrin, and a first hydroporphyrin, optionally wherein the first hydroporphyrin is a chlorin or a bacteriochlorin. In some embodiments, a first hydroporphyrin is between first and second porphyrins. In some embodiments, a second porphyrin is between a first porphyrin and a first hydroporphyrin. In particular embodiments of the invention, the first porphyrin and/or second porphyrin has a structure of Formula ha and/or the first hydroporphyrin has a structure of Formula IIa or Formula IIc. In some embodiments of the invention, the first porphyrin and/or second porphyrin has a structure of Formula ha and/or the first hydroporphyrin has a structure of Formula IIb or Formula IId, optionally wherein M¹ and/or M² is zinc or magnesium. In particular embodiments of the invention, the first porphyrin and/or second porphyrin has a structure of Formula Ib and/or the first hydroporphyrin has a structure of Formula IIb or Formula IId, optionally wherein M¹ and/or M² is zinc or magnesium. In some embodiments of the invention, the first porphyrin and/or second porphyrin has a structure of Formula Ib and/or the first hydroporphyrin has a structure of Formula IIa or Formula IIc.

In some embodiments, a compound of the present invention may comprise a large cluster or an array of porphyrins (e.g., having the structure of Formula Ia or Formula Ib) that is linked to at least one hydroporphyrin (e.g., having the structure of one of Formula IIa-d). For example, in some embodiments, provided is a linear array of porphyrins (e.g., 2, 3, 4, 5, or more), optionally linked at a meso position of the porphyrin ring, with a hydroporphyrin as the acceptor, optionally linked at the beta position of the hydroporphyrin ring. In some embodiments, the hydroporphyrin is at the end (e.g., terminus) of the compound. As another example, a compound may comprise a dendritic cluster (e.g., 4, 5, 6, 7, 8, or more) of porphyrins, optionally linked at a meso position of the porphyrin ring, that is attached to a hydroporphyrin optionally at the center of the compound and optionally linked at a beta position of the hydroporphyrin ring, such that the energy of the porphyrins is funneled to the acceptor hydroporphyrin. In some embodiments, a compound of the present invention comprises a star shaped structure having multiple porphyrins (e.g., 3, 4, 5, or more), optionally linked at a meso position of the porphyrin rings, that is attached to a hydroporphyrin, optionally linked at a beta position of the hydroporphyrin ring, such that the energy of the porphyrins is funneled to the acceptor hydroporphyrin.

In some embodiments, if two or more porphyrins are attached to a hydroporphyrin, the hydroporphyrin is attached to each of the porphyrins at a beta position on the hyrdroporhyrin ring via a direct bond or a linking group. In some embodiments, each of the two or more porphyrins is attached via a direct bond or a linking group to the hydroporphyrin at a meso position of the porphyrin ring.

As used herein, a “linking group”, “linker”, and “attachment moiety” are used interchangeably herein and refer to a functional group that provides a reactive site for conjugation and/or is used to attach (e.g.) two compounds (e.g., is used to and/or facilitates attachment of a porphyrin and a hydroporphyrin). In some embodiments, a linking group attaches a porphyrin to a hydroporphyrin, a porphyrin to another porphyrin or a hydroporphyrin to another hydroporphyrin (collectively, porphyrins and hydroporphyrins may be referred to herein as heterocyclic macrocycles). As described above, in some embodiments, no linking group is present between heterocyclic macrocycles in a compound of the present invention. However, in some embodiments, a compound of the present invention includes a linking group between at least two of the macrocycles (e.g., between the first porphyrin and the first hydroporphyrin) and the linking group attaches the two macrocycles. In some embodiments, the linking group includes at least one substituent (e.g., ethynyl), which may modify an emission wavelength of the compound compared to an emission wavelength of the compound in the absence of the substituent. In some embodiments, the linking group includes at least one site (e.g., functional group or substituent) for bioconjugation.

In some embodiments of the invention, the linking group between two heterocyclic macrocycles (e.g., the first porphyrin and the first hydroporphyrin) is an alkyl (e.g., a C1-C20 alkyl), alkenyl (e.g., a C2-C20 alkenyl), alkynyl (e.g., a C2-C20 alkynyl), cycloalkyl (e.g., a C3-C20 cycloalkyl), aryl, alkenylaryl, alkynylaryl, alkynylalknyl, heterocyclo, heteroaryl, amino, amido, and/or peptidyl group, each of which may be substituted or unsubstituted. In some embodiments, the linking group is a nucleic acid (e.g., RNA and/or DNA such as single stranded DNA (ssDNA)), a polymer, a biomolecule (e.g., a peptide, etc.), alkyl (e.g., a C1-C20 alkyl), alkenyl (e.g., a C2-C20 alkenyl), alkynyl (e.g., a C2-C20 alkynyl), cycloalkyl (e.g., a C3-C20 cycloalkyl), aryl, alkenylaryl, alkynylaryl, alkynylalknyl, heterocyclo, heteroaryl, amino, amido, and any combination thereof. In some embodiments, the linker comprises a nucleic acid, optionally a ssDNA. In some embodiments, the linker is ethyne, ethane, p-phenylene, 4,4′-biphenyl, 4,4″-terphenyl, 1,4-diphenylethyne, phenylethyne, thienyl, or peptidyl group that is optionally substituted or unsubstituted. In some embodiments, the linker is phenylethyne that is optionally substituted or unsubstituted. In some embodiments, a linking group is an aromatic or aliphatic group (which may be substituted or unsubstituted and may optionally contain heteroatoms such as N, O, or S) such as, but are not limited to, aryl, alkyl, heteroaryl, heteroalkyl (e.g., oligoethylene glycol), peptide, and/or polysaccharide linkers.

Energy transfer from a donor to an acceptor in a compound of the present invention may occur through bond (i.e., through bond energy transfer (TBET)) or may occur through space. A linking group between two heterocyclic macrocycles in a compound of the present invention may affect the type of energy transfer. In some embodiments, energy transfer in a compound of the present invention from a donor (e.g., porphyrin) to an acceptor (e.g., hydroporphyrin) occurs via Forster resonance energy transfer (FRET). In some embodiments, energy transfer in a compound of the present invention is via FRET and the compound comprises a linking group that includes at least one, and in some embodiments, at least two, rotatable bonds. In some embodiments, energy transfer in a compound of the present invention from a donor (e.g., porphyrin) to an acceptor (e.g., hydroporphyrin) occurs via Dexter energy transfer. In such cases where Dexter energy transfer occurs in a compound of the present invention, a linker present in the compound may provide for conjugation and/or electron delocalization.

Non-limiting examples of linking groups include:

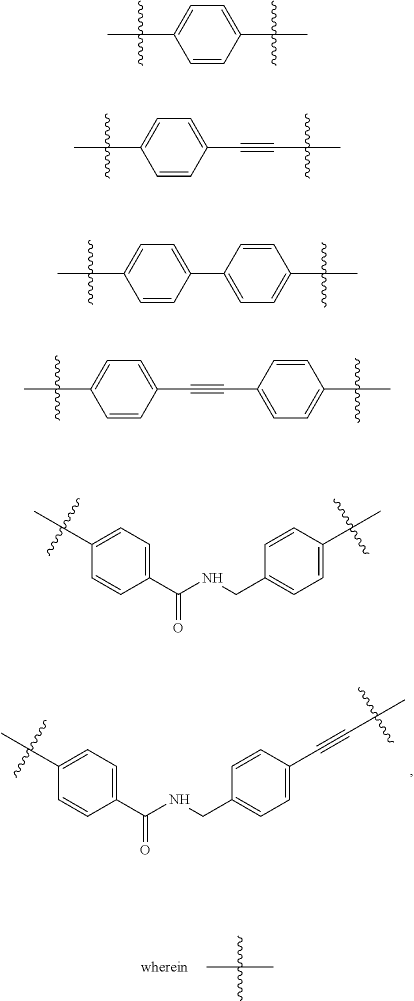

is an attachment point to another compound (e.g., a porphyrin or hydroporphyrin). In some embodiments, the left attachment point in one or more of the above exemplary linkers is attached to a porphyrin and the right attachment point in one or more of the above exemplary linkers is attached to a hydroporphyrin.

In some embodiments, a linking group attaches at least one portion of a compound of the present invention to another compound or object. A “linking group” may connect two or more heterocyclic macrocycles in a compound of the present invention, and, in some embodiments, a linking group may alternatively or in addition be used to connect the compound of the present invention to a different compound or object. For example, a compound of the present invention may include a linking group that provides a reactive site for conjugation so that the compound may be coupled to and/or conjugated to other compounds or groups such as proteins, peptides, targeting agents (e.g., antibodies), polymers, particles (e.g., nanoparticles, organic, polymeric or inorganic beads, other solid support surfaces, etc.) and the like. In some embodiments, these other compounds or groups may be attached to a linking group of the compound of the present invention optionally via linkages that include, for example, aryl, alkyl, heteroaryl, heteroalkyl (e.g., oligoethylene glycol), peptide, polysaccharide functional groups, and the like. The linking group may be simply a reactive attachment group (e.g., —R′ where R′ is a reactive group such as bromo) or may comprise a combination of an intervening group coupled to a reactive group (e.g., —R″R′, where R′ is a reactive group and R′ is an intervening group such as a hydrophilic group). In some embodiments, a compound of the present invention may comprise a first linking group that is attached to at least one heterocylic macrocycle of the compound and to a protein, peptide, targeting agent (e.g., antibody), polymer, or particle (e.g., nanoparticle, organic, polymeric or inorganic bead, other solid support surface, etc.), and the compound may optionally comprise a second linking group that attaches two heterocylic macrocycles.

For bioconjugation purposes, the choice of water-solubilizing group(s) and conjugation groups may be made so as to achieve orthogonal coupling. For example, if a carboxylic acid is used for water solubility, an aldehyde might be used for bioconjugation (via reductive amination with an amino-substituted biomolecule). If a carboxylic acid is used for bioconjugation (via carbodiimide-activation and coupling with an amino-substituted biomolecule), then a complementary group may be used for water solubility (e.g., sulfonic acid, guanidinium, pyridinium). Bioconjugatable groups include, but are not limited to, carboxylic acids or esters thereof, amines (including amine derivatives) such as isocyanates, isothiocyanates, iodoacetamides, azides, diazonium salts, etc., acids or acid derivatives such as N-hydroxysuccinimide esters (more generally, active esters derived from carboxylic acids; e.g., p-nitrophenyl ester), acid hydrazides, etc., and other linking groups such as aldehydes, sulfonyl chlorides, sulfonyl hydrazides, epoxides, hydroxyl groups, thiol groups, maleimides, aziridines, acryloyls, halo groups, biotin, 2-iminobiotin, etc. Linking groups such as the foregoing are known and described in U.S. Pat. Nos. 6,728,129; 6,657,884; 6,212,093; and 6,208,553.

Other functional groups that may be attached to a compound of the present invention (e.g., a compound that includes a porphyrin of Formula Ia or Ib attached to a hydroporphyrin of Formula IIa-IId) and/or that may tune or adjust the solubility properties of the compound include, but are not limited to, hydrophobic groups, hydrophilic groups, polar groups, and/or amphipathic groups. Polar groups include carboxylic acid, sulfonic acid, guanidinium, carbohydrate, hydroxy, amino acid, pyridinium, imidazolium, etc. Such groups may be attached to substituents that are linear or branched alkyl (e.g., swallowtail), aryl, heteroaryl, heteroalkyl (e.g., oligoethylene glycol), peptide, polysaccharide, etc. Suitable hydrophilic groups may include polyols or polyalkylene oxide groups, including straight and/or branched-chain polyols, with particular examples including, but not limited to, poly(propylene glycol), polyethylene-polypropylene glycol, and/or poly(ethylene glycol). In some embodiments, the hydrophilic group may have a number average molecular weight of 20,000 to 40,000 or 60,000. Suitable hydrophilic groups and the manner of coupling thereof are known and described in, for example, U.S. Pat. Nos. 4,179,337; 5,681,811; 6,524,570; 6,656,906; 6,716,811; and 6,720,306. For example, compounds can be pegylated using a single 40,000 molecular weight polyethylene glycol moiety that is attached to a compound of the present invention. Suitable hydrophilic groups also include linear or branched alkyl groups substituted with ionic or polar groups, examples of which include but are not limited to swallowtail groups such as described in Borbas and Lindsey, U.S. Pat. No. 8,530,459. In some embodiments, a hydrophilic group may be coupled at one or more sites of a porphyrin or hydroporphyrin of the invention, e.g., covalently coupled thereto, to facilitate delivery thereof, or improve stability, in accordance with known techniques (e.g., to the N-terminus of the peptide).

Targeting groups include biomolecules such as antibodies, proteins, peptides, and nucleic acids, each of which may be attached by means of a linking group. Particles such as nanoparticles, glass beads, etc., may be attached by means of a linking group. Where such additional compounds are attached to a compound of the invention it may be directly to the compound or attached by means of an intervening group such as a linker or hydrophilic group.

In some embodiments of the invention, a compound of the invention further includes an auxochrome, optionally wherein the auxochrome is attached to an atom of a porphyrin (e.g., a first porphyrin) and/or to an atom of a hydroporphyrin (e.g., a first hydroporphyrin) present in the compound. While any of the substituents described with reference to Formula Ia-b and Formula IIa-d may be auxochromes, in some embodiments of the invention, the auxochrome is an acyl, acyloxy, ester (e.g., alkyloxycarbonyl or aryloxycarbonyl), carboxylic acid, cyano, sulfonyl, sulfoxyl, alkene, alkyne, arene, amino, nitro, hydroxy, mercapto, and/or alkoxy group that is optionally substituted or unsubstituted. In some embodiments, auxochromes may be on any meso- or 3-site on the perimeter of the heterocyclic macrocycle. In some embodiments, an auxochrome may be present on the 2, 3, 12, and/or 13 positions of the hydroporphyrin.

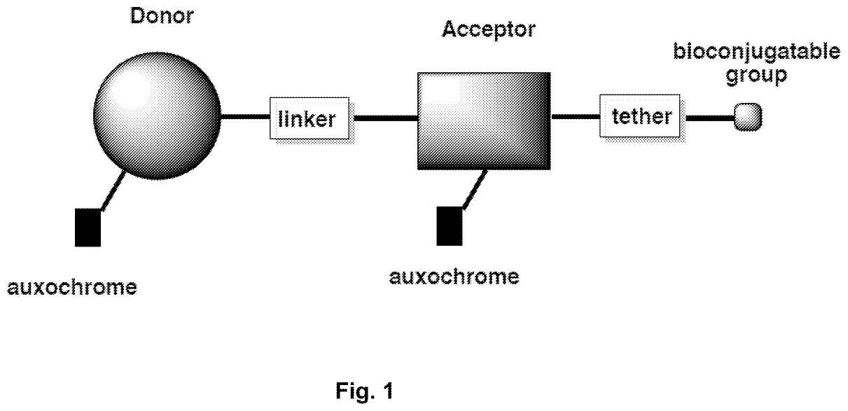

In some embodiments, a compound of the invention may have the general structure shown in FIG. 1, wherein the donor is at least one porphyrin (e.g., having Formula Ia or Ib) and the acceptor is a hydroporphyrin (e.g., having Formula IIa-IId) and the tether is a linking group that provides space (distance) between the hydroporphyrin and the bioconjugatable group.

A surface attachment group may be a reactive group coupled directly to a porphyrin or hydroporphyrin or coupled to a porphyrin or hydroporphyrin by means of an intervening linker. A surface attachment group may be in protected or unprotected form. Linking groups that link to a surface attachment group may include, for example, aryl, alkyl, alkenyl, alkynyl, heteroaryl, heteroalkyl, oligoethylene glycol (e.g., PEG), peptide, polysaccharide, etc. Examples of surface attachment groups (with the reactive site or group in unprotected form) include, but are not limited to, alkene, alkyne, alcohol, thiol, selenyl, phosphono, telluryl, cyano, amino, formyl, halo, boryl, and carboxylic acid surface attachment groups such as:
4-carboxyphenyl, carboxymethyl, 2-carboxyethyl, 3-carboxypropyl, 2-(4-carboxyphenyl)ethynyl, 4-(2-(4-carboxyphenyl)ethynyl)phenyl, 4-carboxymethylphenyl, 4-(3-carboxypropyl)phenyl, 4-(2-(4-carboxymethylphenyl)ethynyl)phenyl; 4-hydroxyphenyl, hydroxymethyl, 2-hydroxyethyl, 3-hydroxypropyl, 2-(4-hydroxyphenyl)ethynyl, 4-(2-(4-hydroxyphenyl)ethynyl)phenyl, 4-hydroxymethylphenyl, 4-(2-hydroxyethyl)phenyl, 4-(3-hydroxypropyl)phenyl, 4-(2-(4-hydroxymethylphenyl)ethynyl)phenyl; 4-mercaptophenyl, mercaptomethyl, 2-mercaptoethyl, 3-mercaptopropyl, 2-(4-mercaptophenyl)ethynyl, 4-(2-(4-mercaptophenyl)ethynyl)phenyl, 4-mercaptomethylphenyl, 4-(2-mercaptoethyl)phenyl, 4-(3-mercaptopropyl)phenyl, 4-(2-(4-mercaptomethylphenyl)ethynyl)phenyl; 4-selenylphenyl, selenylmethyl, 2-selenylethyl, 3-selenylpropyl, 2-(4-selenylphenyl)ethynyl, 4-selenylmethylphenyl, 4-(2-selenylethyl)phenyl, 4-(3-selenylpropyl)phenyl, 4-selenylmethylphenyl, 4-(2-(4-selenylphenyl)ethynyl)phenyl; 4-tellurylphenyl, tellurylmethyl, 2-tellurylethyl, 3-tellurylpropyl, 2-(4-tellurylphenyl)ethynyl, 4-(2-(4-tellurylphenyl)ethynyl)phenyl, 4-tellurylmethylphenyl, 4-(2-tellurylethyl)phenyl, 4-(3-tellurylpropyl)phenyl, 4-(2-(4-tellurylmethylphenyl)ethynyl)phenyl;
4-(dihydroxyphosphoryl)phenyl, (dihydroxyphosphoryl)methyl, 2-(dihydroxyphosphoryl) ethyl, 3-(dihydroxyphosphoryl)propyl, 2-[4-(dihydroxyphosphoryl)phenyl]ethynyl, 4-[2-[4-(dihydroxyphosphoryl)phenyl]ethynyl]phenyl, 4-[(dihydroxyphosphoryl)methyl]phenyl, 4-[2-(dihydroxyphosphoryl)ethyl]phenyl, 4-[2-[4-(dihydroxyphosphoryl)methylphenyl]ethynyl]phenyl; 4-(hydroxy(mercapto)phosphoryl)phenyl, (hydroxy(mercapto)phosphoryl)methyl, 2-(hydroxy(mercapto)phosphoryl)ethyl, 3-(hydroxy(mercapto)phosphoryl)propyl, 2-[4-(hydroxy(mercapto)phosphoryl)phenyl]ethynyl, 4-[2-[4-(hydroxy(mercapto)phosphoryl)phenyl]ethynyl]phenyl, 4-[(hydroxy(mercapto)phosphoryl)methyl]phenyl, 4-[2-(hydroxy(mercapto)phosphoryl)ethyl]phenyl, 4-[2-[4-(hydroxy(mercapto)phosphoryl)methylphenyl]ethynyl]phenyl;
4-cyanophenyl, cyanomethyl, 2-cyanoethyl, 3-cyanopropyl, 2-(4-cyanophenyl)ethynyl, 4-[2-(4-cyanophenyl)ethynyl]phenyl, 4-(cyanomethyl)phenyl, 4-(2-cyanoethyl)phenyl, 4-[2-[4-(cyanomethyl)phenyl]ethynyl]phenyl;
4-cyanobiphenyl; 4-aminophenyl, aminomethyl, 2-aminoethyl, 3-aminopropyl, 2-(4-aminophenyl)ethynyl, 4-[2-(4-aminophenyl)ethynyl]phenyl, 4-aminobiphenyl;
4-formylphenyl, 4-bromophenyl, 4-iodophenyl, 4-vinylphenyl, 4-ethynylphenyl, 4-allylphenyl, 4-[2-(trimethylsilyl)ethynyl]phenyl, 4-[2-(triisopropylsilyl)ethynyl]phenyl, 4-(4,4,5,5-tetramethyl-1,3,2-dioxaborolan-2-yl)phenyl;
formyl, bromo, iodo, bromomethyl, chloromethyl, ethynyl, vinyl, allyl; 4-(ethynyl)biphen-4□-yl, 4-[2-(triisopropylsilyl)ethynyl]biphen-4□-yl, 3,5-diethynylphenyl;
4-(bromomethyl)phenyl, and 2-bromoethyl.

In addition to the monodentate linker-surface attachment groups described above, multidentate linkers may be employed [Nikitin, K. Chem. Commun. 2003, 282-283; Hu, J.; Mattern, D. L. J. Org. Chem. 2000, 65, 2277-2281; Yao, Y.; Tour, J. M. J. Org. Chem. 1999, 64, 1968-1971; Fox, M. A. et al. Langmuir, 1998, 14, 816-820; Galoppini, E.; Guo, W. J. Am. Chem. Soc. 2001, 123, 4342-4343; Deng, X. et al. J. Org. Chem. 2002, 67, 5279-5283; Hector Jr., L. G. et al. Surface Science, 2001, 494, 1-20; Whitesell, J. K.; Chang, H. K. Science, 1993, 261, 73-76; Galoppini, E. et al. J. Am. Chem. Soc. 2002, 67, 7801-7811; Siiman, O. et al. Bioconjugate Chem. 2000, 11, 549-556]. Tripodal linkers bearing thiol, carboxylic acid, alcohol, or phosphonic acid units are particularly attractive for firmly anchoring a molecular device on a planar surface. Specific examples of such linkers are built around the triphenylmethane or tetraphenylmethane unit, including the following:
1,1,1-tris[4-(S-acetylthiomethyl)phenyl]methyl,
4-{1,1,1-tris[4-(S-acetylthiomethyl)phenyl]methyl}phenyl,
1,1,1-tris[4-(dihydroxyphosphoryl)phenyl]methyl,
4-{1,1,1-tris[4-(dihydroxyphosphoryl)phenyl]methyl}phenyl,
1,1,1-tris[4-dihydroxyphosphorylmethyl)phenyl]methyl, and
4-{1,1,1-tris[4-(dihydroxyphosphorylmethyl)phenyl]methyl}phenyl;
   All as described in Balakumar, Muthukumaran and Lindsey, U.S. patent application Ser. No. 10/867,512 (filed Jun. 14, 2004). See also Lindsey, Loewe, Muthukumaran, and Ambroise, US Patent Application Publication No. 20050096465 (Published May 5, 2005), particularly paragraph 51 thereof. Additional examples of multidentate linkers include but are not limited to:
   Alkene surface attachment groups (2, 3, 4 carbons) such as:
3-vinylpenta-1,4-dien-3-yl,
4-(3-vinylpenta-1,4-dien-3-yl)phenyl,
4-(3-vinylpenta-1,4-dien-3-yl)biphen-4′-yl,
4-allylhepta-1,6-dien-4-yl,
4-(4-allylhepta-1,6-dien-4-yl)phenyl,
4-(4-allylhepta-1,6-dien-4-yl)biphen-4′-yl,
5-(1-buten-4-yl)nona-1,8-dien-5-yl, 4-[5-(1-buten-4-yl)nona-1,8-dien-5-yl]phenyl, 4-[5-(1-buten-4-yl)nona-1,8-dien-5-yl]biphen-4′-yl, etc.
   Alkyne surface attachment groups (2, 3, 4 carbons) such as:
3-ethynylpenta-1,4-diyn-3-yl,
4-(3-ethynylpenta-1,4-diyn-3-yl)phenyl,
4-(3-ethynylpenta-1,4-diyn-3-yl)biphen-4′-yl,
4-propargylhepta-1,6-diyn-4-yl,
4-(4-propargylhepta-1,6-diyn-4-yl)phenyl,
4-(4-propargylhepta-1,6-diyn-4-yl)biphen-4-yl,
5-(1-butyn-4-yl)nona-1,8-diyn-5-yl,
4-[5-(1-butyn-4-yl)nona-1,8-diyn-5-yl]phenyl,
4-[5-(1-butyn-4-yl)nona-1,8-diyn-5-yl]biphen-4-yl,
   Alcohol surface attachment groups (1, 2, 3 carbons), such as:
2-(hydroxymethyl)-1,3-dihydroxyprop-2-yl,
4-[2-(hydroxymethyl)-1,3-dihydroxyprop-2-yl]phenyl,
4-[2-(hydroxymethyl)-1,3-dihydroxyprop-2-yl]biphen-4-yl,
3-(2-hydroxyethyl)-1,5-dihydroxypent-3-yl,
4-[3-(2-hydroxyethyl)-1,5-dihydroxypent-3-yl]phenyl,
4-[3-(2-hydroxyethyl)-1,5-dihydroxypent-3-yl]biphen-4-yl,
4-(3-hydroxypropyl)-1,7-dihydroxyhept-4-yl,
4-[4-(3-hydroxypropyl)-1,7-dihydroxyhept-4-yl]phenyl,
4-[4-(3-hydroxypropyl)-1,7-dihydroxyhept-4-yl]biphen-4-yl, etc.,
   Thiol surface attachment groups (1, 2, 3 carbons) such as:
2-(mercaptomethyl)-1,3-dimercaptoprop-2-yl,
4-[2-(mercaptomethyl)-1,3-dimercaptoprop-2-yl]phenyl,
4-[2-(mercaptomethyl)-1,3-dimercaptoprop-2-yl]biphen-4-yl,
3-(2-mercaptoethyl)-1,5-dimercaptopent-3-yl
4-[3-(2-mercaptoethyl)-1,5-dimercaptopent-3-yl]phenyl,
4-[3-(2-mercaptoethyl)-1,5-dimercaptopent-3-yl]biphen-4-yl,
4-(3-mercaptopropyl)-1,7-dimercaptohept-4-yl,
4-[4-(3-mercaptopropyl)-1,7-dimercaptohept-4-yl]phenyl,
4-[4-(3-mercaptopropyl)-1,7-dimercaptohept-4-yl]biphen-4-yl etc.,
   Selenyl surface attachment groups (1, 2, 3 carbons), such as:
2-(selenylmethyl)-1,3-diselenylprop-2-yl,
4-[2-(selenylmethyl)-1,3-diselenylprop-2-yl]phenyl,
4-[2-(mercaptomethyl)-1,3-dimercaptoprop-2-yl]biphen-4-yl,
3-(2-selenylethyl)-1,5-diselenylpent-3-yl,
4-[3-(2-selenylethyl)-1,5-diselenylpent-3-yl]phenyl,
4-[3-(2-selenylethyl)-1,5-diselenylpent-3-yl]biphen-4-yl,
4-(3-selenylpropyl)-1,7-diselenylhept-4-yl,
4-[4-(3-selenylpropyl)-1,7-diselenylhept-4-yl]phenyl,
4-[4-(3-selenylpropyl)-1,7-diselenylhept-4-yl]biphen-4-yl, etc.
   Phosphono surface attachment groups (1, 2, 3 carbons), such as:
2-(phosphonomethyl)-1,3-diphosphonoprop-2-yl,
4-[2-(phosphonomethyl)-1,3-diphosphonoprop-2-yl]phenyl,
4-[2-(phosphonomethyl)-1,3-diphosphonoprop-2-yl]biphen-4□-yl,
3-(2-phosphonoethyl)-1,5-diphosphonopent-3-yl,
4-[3-(2-phosphonoethyl)-1,5-diphosphonopent-3-yl]phenyl,
4-[3-(2-phosphonoethyl)-1,5-diphosphonopent-3-yl]biphen-4-yl,
4-(3-phosphonopropyl)-1,7-diphosphonohept-4-yl,
4-[4-(3-phosphonopropyl)-1,7-diphosphonohept-4-yl]phenyl,
4-[4-(3-phosphonopropyl)-1,7-diphosphonohept-4-yl]biphen-4-yl, etc., and
   Carboxylic acid surface attachment groups (1, 2, 3 carbons), such as:
2-(carboxymethyl)-1,3-dicarboxyprop-2-yl,
4-[2-(carboxymethyl)-1,3-dicarboxyprop-2-yl]phenyl,
4-[2-(carboxymethyl)-1,3-dicarboxyprop-2-yl]biphen-4-yl,
3-(2-carboxyethyl)-1,5-dicarboxypent-3-yl,
4-[3-(2-carboxyethyl)-1,5-dicarboxypent-3-yl]phenyl,
4-[3-(2-carboxyethyl)-1,5-dicarboxypent-3-yl]biphen-4-yl,
4-(3-carboxypropyl)-1,7-dicarboxyhept-4-yl,
4-[4-(3-carboxypropyl)-1,7-dicarboxyhept-4-yl]phenyl,
4-[4-(3-carboxypropyl)-1,7-dicarboxyhept-4-yl]biphen-4-yl, etc.

It is to be understood that the compounds provided herein may contain chiral centers. Such chiral centers may be of either the (R) or (S) configuration or may be a mixture thereof. Thus, the compounds provided herein may be enantiomerically pure, or be stereoisomeric or diastereomeric mixtures. It is to be understood that the chiral centers of the compounds provided herein may undergo epimerization in vivo. As such, one of skill in the art will recognize that administration of a compound in its (R) form is equivalent, for compounds that undergo epimerization in vivo, to administration of the compound in its (S) form.

In some embodiments of the invention, a compound of the invention further includes at least one additional chromophore, optionally wherein the at least one additional chromophore is a perylene, carotenoid, dipyrrinatoborondifluoride, or bis(dipyrrinato)metal complex.

Also provided according to embodiments of the invention is a particle that includes a compound of the invention. In some embodiments, the particle is a microparticle or a nanoparticle. Also provided herein is a plurality of such particles.

In some embodiments, the particle includes a shell and a core. In some embodiments, a compound of the present invention is present in the core. In some embodiments, a compound of the present invention is encapsulated in a polymer and the polymer forms the shell, optionally wherein the polymer comprises one or more hydrophobic unit(s) and one or more hydrophilic unit(s), and optionally comprises a bioconjugate group. In some embodiments, the particle maintains a compound of the invention in a non-aggregated state. In some embodiments, foldamers, or single chain nanoparticles (SCNPs), may be used to encapsulate or contain a compound of the present invention. A compound of the present invention may be used as the dye and/or as the acceptor dye and donor luminophore(s) of the polymers and/or particles described in U.S. Application Publication No. 2020/0385583, International Application No. PCT/US19/054008, and International Application No. PCT/US20/61285, which are incorporated herein by reference in their entirety. In some embodiments, a polymer and/or particle of the present invention has a structure as described in U.S. Application Publication No. 2020/0385583, International Application No. PCT/US19/054008, and International Application No. PCT/US20/61285, which are incorporated herein by reference in their entirety.

In particular embodiments of the invention, a particle of the invention includes a compound of the invention attached to a polymer so that the resulting compound has the structure of Formula IIIa or Formula IIIb:

A-B—C (IIIa), or

C-A-B (IIIb)
wherein A is the compound of the present invention; B is the polymer; and C is an optional bioconjugate group. In some embodiments, C is not present in the particle (i.e., the particle is devoid of a bioconjugate group), so the particle has a structure of A-B, wherein A is the compound of the present invention and B is the polymer. In some embodiments, C is present in the particle. In some embodiments, the polymer B has a molecular weight in a range of about 1,000 Da to about 175,000 Da, including a molecular weight of about 1,000, 5,000, 10,000, 20,000, 30,000, 40,000, 50,000, 60,000, 70,000, 80,000, 90,000, 100,000, 110,000, 120,000, 130,000, 140,000, 150,000, 160,000, 170,000, 175,000 Da, and any range defined therebetween.

In some embodiments of the invention, a compound of the present invention is attached to a surface of a particle (e.g., a nanoparticle). In some embodiments, the particle includes polystyrene and/or silica. In some embodiments, a compound of the present invention is attached to a particle and/or bead as described in U.S. Patent Application Publication No. 2019/0264102, which is incorporated herein by reference in its entirety.

In some embodiments of the invention, a particle of the present invention is soluble in water or an aqueous solution. In particular embodiments, a particle of the present invention has a solubility in water at room temperature in a range of about 1 mg/mL to about 10, 50, or 100 mg/mL or more (e.g., 1, 2, 3, 4, 5, 6, 7, 8, 9, 10, 20, 30, 40, 50, 60, 70, 80, 90, or 100 mg/L, or any range defined therebetween).

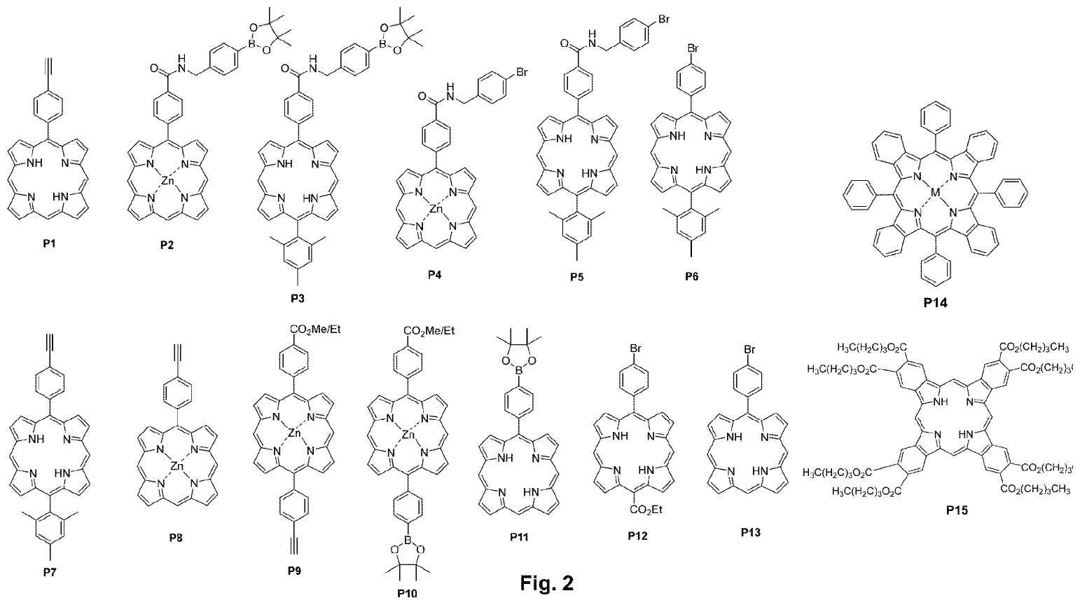

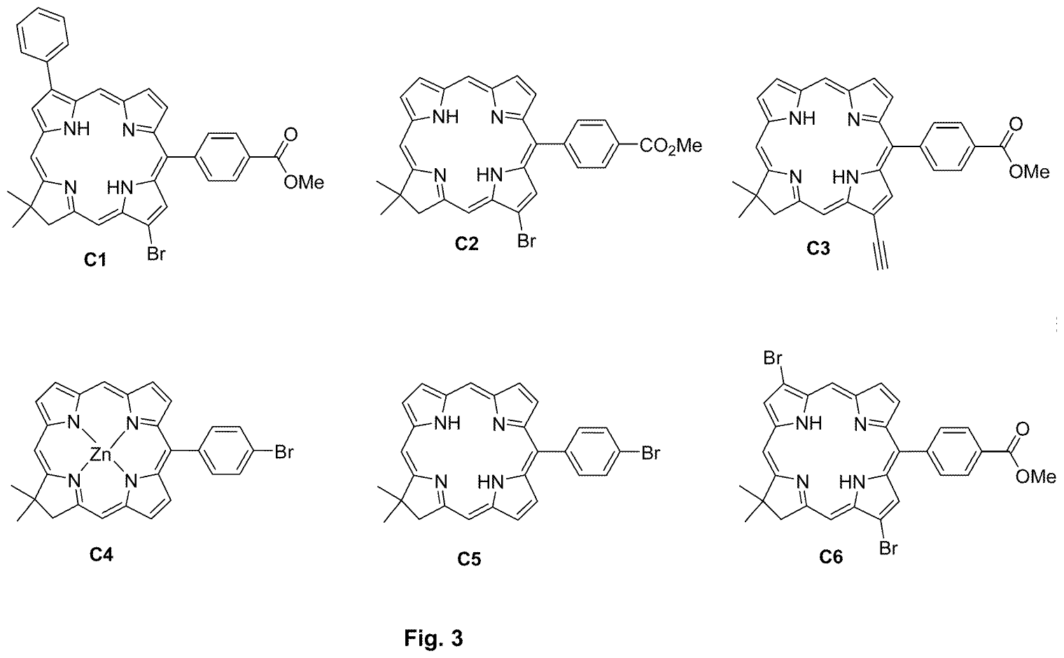

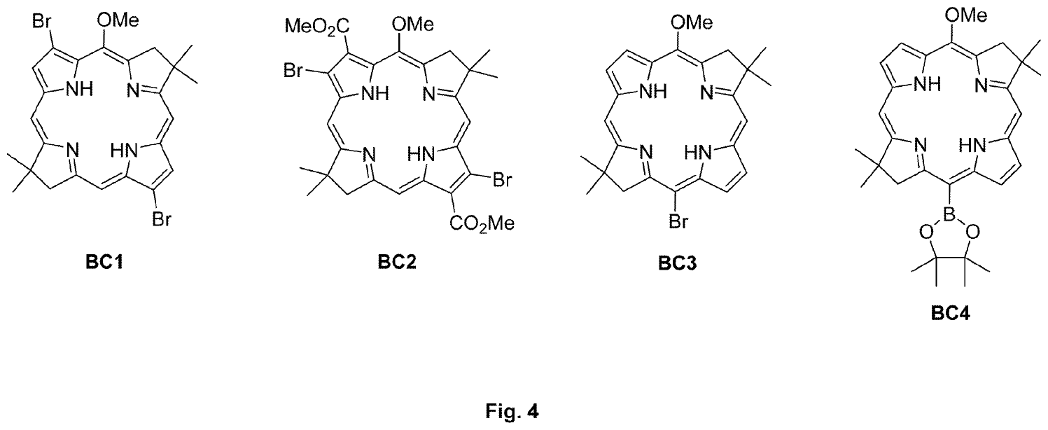

A method of the present invention may provide for the synthesis of a compound of the invention. In some embodiments, at least one porphyrin of Formula Ia-Ib and at least one hydroporphyrin of Formula IIa-IId may be reacted to attach the at least two compounds to thereby form a compound of the invention. In some embodiments, an intermediate used in forming a compound of the present invention may have a structure as shown in FIG. 2, which shows exemplary porphyrins optionally including one or more substituent(s) (e.g., an auxochrome, linking group, bioconjugatable group, etc.). In some embodiments, an intermediate used in forming a compound of the present invention may have a structure as shown in FIG. 3 and FIG. 4, which show exemplary chlorins and bacteriochlorins, respectively, optionally including one or more substituent(s) (e.g., an auxochrome, linking group, bioconjugatable group, etc.).

General methods of porphyrin and hydroporphyrin synthesis, including incorporation of functional groups on the ring, are known in the art. Examples include, but are not limited to, compounds and methods described in U.S. Pat. Nos. 8,097,609, 10,919,904, and 10,836,774 and International Application Publication Nos. WO2020/236828 and WO2020/236818, which are incorporated herein by reference in their entirety. A porphyrin having a first substituent or group thereon may be reacted with a hydroporphyrin having a second substituent or group thereon that is reactive with the first substituent or group of the porphyrin. In some embodiments, the first substituent or group, the second substituent or group, or a reaction product thereof, once reacted, forms a direct bond or linking group between the porphyrin and hydroporphyrin. Any of the linking groups described herein may be formed in this manner. The linking groups, attachment groups, bioconjugatable groups, and targeting groups may be added prior to attaching any two of the heterocyclic macrocycles, during the attachment of heterocyclic macrocycles, and/or after attaching such heterocyclic macrocycles.

The methods and intermediates described herein may be useful for the synthesis of compounds, as described herein. Such compounds may be useful per se or in further modified form (e.g., as a salt, metalated compound, conjugate, and/or prodrug) for diagnostic and/or therapeutic purposes in like manner as other compounds described for photodynamic therapy, such as described in US Patent Application Publication No. 2004/0044197 to Pandey et al. and as set forth in further detail below. In some embodiments, a compound of the present invention may be used in an application where wavelength tuning and/or bioconjugation is utilized and/or sought. A method and/or compound of the present invention may provide for one or more (e.g., 1, 2, 3, 4, 5, or more) different substituents to be attached at one or more (e.g., 1, 2, 3, 4, 5, or more) locations on a compound of the present invention (e.g., on the AD half, but not the BC half or vice versa), which may be advantageous in applications including, but not limited to, wavelength tuning and/or bioconjugation.

The compounds of the invention may have desirable photophysical properties. In some embodiments of the invention, for a compound of the invention comprising a porphyrin (e.g., a first porphyrin) and a hydroporphyrin (e.g., a first hydroporphyrin), the lowest-energy singlet excited state of the porphyrin is greater than the lowest-energy singlet excited state of the hydroporphyrin. This facilitates singlet energy transfer from the porphyrin to the hydroporphyrin. In some embodiments, the energy transfer pathway is sufficiently effective that it dominates over intrinsic excited state de-excitation pathways of the porphyrin, including fluorescence emission. Instead, fluorescence occurs from the acceptor hydroporphyrin after receiving the energy transfer from the porphyrin. Thus, in some embodiments, the fluorescence characteristics of the compound may not be substantially affected by the porphyrin.

In some embodiments, a compound of the present invention is excited at a wavelength in the violet region of the visible light spectrum. In some embodiments, a compound of the present invention is excited at a wavelength in a range of about 350 nm to about 500, 600, 700, 800, 900, 1000, 1100, 1200, or 1300 nm, including at about 350, 360, 370, 380, 390, 400, 410, 420, 430, 440, 450, 460, 470, 480, 490, 500, 600, 700, 800, 900, 1000, 1100, 1200, or 1300 nm, and any range defined therebetween. A compound of the present invention may be excited with a laser (e.g., a laser emitting a wavelength in a range of about 350 nm to about 500, 600, 700, 800, 900, 1000, 1100, 1200, or 1300 nm). In some embodiments, a compound of the present invention is excited at a wavelength in a range of about 350 nm to about 500 nm. In some embodiments, a compound of the present invention is excited at a wavelength in a range of about 375 nm to about 440 nm. In some embodiments, a compound of the present invention is excited at a wavelength of about 405 nm. In some embodiments, a compound of the present invention is excited at a wavelength of about 445 nm. In some embodiments, a compound of the present invention is excited with a violet laser, optionally at a wavelength of about 407 nm. In some embodiments, a compound of the present invention is excited at a wavelength for use in flow cytometry and/or is used in a flow cytometry. In some embodiments, a compound of the present invention is exposed to light having a wavelength in a range of about 675 nm to about 1300 nm and/or may be used in and/or for photoacoustic imaging.

In some embodiments, a compound of the present invention emits light at a wavelength in the red and/or near-infrared region of the visible light spectrum. In some embodiments, a compound of the present invention emits light (e.g., a maximum emission) at a wavelength in a range of about 610 nm to about 2500 nm, and in some cases, in a range of about 610 or 625 nm to about 810 nm. In some embodiments, a compound of the present invention emits light at a wavelength in a range of about 610, 620, 630, 640, 650, 660, 670, 680, 690, 700, 710, 720, 730, 740, 750, 760, 770, 780, 790, 800, 810, 850, 900, 950, 1000, 1250, 1500, 1750, 2000, 2250, or 2500 nm, and any range defined therebetween. In some embodiments, a compound of the present invention emits heat, which may be detected by methods known in the art (e.g., using ultrasound and/or photoacoustic imaging).

In some embodiments, a compound of the present invention comprises a first porphyrin and a first hydroporphyrin and the compound has a brightness that is greater than the brightness of the first porphyrin alone and/or greater than the brightness of the first hydroporphyrin alone, optionally wherein the brightness of the compound is greater than the sum of the brightness of the first porphyrin and the first hydroporphyrin. In some embodiments, a compound of the present invention comprises a first porphyrin and a first hydroporphyrin and the compound has a brightness that is increased compared to the brightness of the first hydroporphyrin alone, optionally wherein the brightness of the compound is increased by about 2, 4, 6, 8, 10, 20, 30, 40, 50, 60, 70, 80, 90, 100 times or more compared to the brightness of the hydroporphyrin alone. As used herein, the term “brightness” refers to the product of molar absorption coefficient and fluorescence quantum yield (ε×ϕ). See, e.g., Lavis D L, Raines R T (2008) “Bright Ideas for Chemical Biology” ACS Chem. Biol. 3:142-155.

In some embodiments, a compound of the present invention has a brightness at the absorbance maximum in the range of about 10,000 M⁻¹cm⁻¹ to about 110,000, 200,00, 300,00, 400,000 or 500,000 M⁻¹cm⁻¹, including 10,000, 11,000, 12,000, 13,000, 14,000, 15,000, 20,000, 30,000, 40,000, 50,000, 60,000, 70,000, 80,000, 90,000, 100,000, 150,000, 200,000, 250,000, 300,000, 350,000, 400,000, 450,000, or 500,000 M⁻¹cm⁻¹, and any range defined therebetween. In some embodiments, a compound of the present invention has a brightness at 405 nm excitation in a range of about 8,000 M⁻¹cm⁻¹ to about 90,000 M⁻¹cm⁻¹, such as at about 8,000, 9,000, 10,000, 15,000, 20,000, 25,000, 30,000, 35,000, 40,000, 45,000, 50,000, 55,000, 60,000, 65,000, 70,000, 75,000, 80,000, 85,000, 88,000, or 90,000, 100,000, 150,000, 200,000, 250,000, 300,000, 350,000, 400,000, 450,000, or 500,000 M⁻¹cm⁻¹, and any range defined therebetween.

In some embodiments, a compound of the present invention comprises a first porphyrin and a first hydroporphyrin and the compound has an emission wavelength for the first porphyrin that is reduced compared to the emission wavelength of the first porphyrin alone or the emission wavelength for the first porphyrin is absent.

In some embodiments, a compound of the present invention comprises a first porphyrin and a first hydroporphyrin and the compound has a fluorescence quantum yield of energy transfer from the first porphyrin to the first hydroporphyrin of at least 50%, 60%, 70%, 80%, 90%, or 95%, optionally wherein an emission wavelength for the first hydroporphyrin is different than and/or distinguishable from an emission wavelength of the first porphyrin.

In some embodiments, a compound of the present invention has an energy transfer from the first porphyrin to the first hydroporphyrin that is about 100 picoseconds or less.

In some embodiments, the first porphyrin has an emission peak having a first intensity and the first hydroporphyrin has an emission peak having a second intensity and the first intensity is 5% or less than the second intensity.

Surprisingly, in some embodiments, compounds of the invention show a significant reduction in the absorbance and/or emission between the absorbance and emission peaks (“middle bands”), which may be advantageous for certain uses, such as multiplex applications. In some embodiments, the compound comprises a first porphyrin and a first hydroporphyrin and the compound has an absorption and emission spectra comprising an emission peak from the first hydroporphyrin having a second intensity and between the excitation wavelength of the compound and the emission peak there is no additional emission peak or no emission peak having an intensity greater than the second intensity.

In some embodiments, the compounds of the invention surprisingly show relatively distinct emission bands. In some embodiments, the first hydroporphyrin of the compound has an emission wavelength with a full width half maximum in a range of about 10 to about 50 nm, including 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, and any range defined therebetween. In some embodiments, the full width half maximum for the compound is in a range of about 14 to about 31 nm. Full width half maximum is the distance between the rise of the peak at half of the maximum amplitude and fall of the peak at half of the maximum amplitude.

In some embodiments, the first porphyrin alone has a molar absorption coefficient of at least about 200,000 M⁻¹cm⁻¹ (including at least about 220,000, 250,000, 300,000, and 350,000 M⁻¹cm⁻¹) at maximum absorbance. In some embodiments, the first porphyrin alone has a molar absorption coefficient of at least about 200,000 M⁻¹cm⁻¹ (including at least about 205,000, 220,000, 230,000, and 250,000 M⁻¹cm⁻¹) at 405 nm.

In some embodiments, a compound of the present invention has a molar absorption coefficient and/or fluorescence quantum yield that is greater than the molar absorption coefficient and/or fluorescence quantum yield, respectively, of the first hydroporphyrin alone. In some embodiments, the compound has a molar absorption coefficient at maximum absorbance in a range of about 120,000 M⁻¹cm⁻¹ to about 450,000, 750,000, 1,000,000, or 1,250,000 M⁻¹cm⁻¹, including 124,000, 130,000, 140,000, 150,000, 160,000, 170,000, 180,000, 190,000, 200,000, 210,000, 220,000, 230,000, 240,000, 250,000, 260,000, 270,000, 280,000, 290,000, 300,000, 310,000, 320,000, 330,000, 340,000, 350,000, 360,000, 370,000, 380,000, 390,000, 400,000, 410,000, 420,000, 430,000, 440,000, 450,000, 500,000, 550,000, 600,000, 650,000, 700,000, 750,000, 800,000, 850,000, 900,000, 950,000, 1,000,000, 1,100,000, 1,200,000, or 1,250,000 M⁻¹cm⁻¹ and any range defined therebetween. A compound of the present invention can be placed into a solution to determine its peak molar absorption coefficient at the indicated wavelength; and the compound may exhibit additional peaks outside of this range, or multiple peaks within this range. In some embodiments, a compound of the present invention has a molar absorption coefficient at a wavelength of about 350 or 375 nm to about 440 or 500 nm in a range of about 110,000 M⁻¹cm⁻¹ to about 350,000, 450,000, 750,000, 1,000,000, or 1,250,000 M⁻¹cm⁻¹, including 115,000, 120,000, 130,000, 140,000, 150,000, 160,000, 170,000, 180,000, 190,000, 200,000, 210,000, 220,000, 230,000, 240,000, 250,000, 260,000, 270,000, 280,000, 290,000, 300,000, 310,000, 320,000, 330,000, 340,000, 350,000, 360,000, 370,000, 380,000, 390,000, 400,000, 410,000, 420,000, 430,000, 440,000, 450,000, 500,000, 550,000, 600,000, 650,000, 700,000, 750,000, 800,000, 850,000, 900,000, 950,000, 1,000,000, 1,100,000, 1,200,000, or 1,250,000 M⁻¹cm⁻¹ and any range defined therebetween. In some embodiments, a compound of the present invention has a molar absorption coefficient at 405 nm in a range of about 110,000 M⁻¹cm⁻¹ to about 350,000, 380,000, 450,000, 750,000, 1,000,000, or 1,250,000 M⁻¹cm⁻¹, including 115,000, 120,000, 130,000, 140,000, 150,000, 160,000, 170,000, 180,000, 190,000, 200,000, 210,000, 220,000, 230,000, 240,000, 250,000, 260,000, 270,000, 280,000, 290,000, 300,000, 310,000, 320,000, 330,000, 340,000, 350,000, 360,000, 370,000, 380,000, 390,000, 400,000, 410,000, 420,000, 430,000, 440,000, 450,000, 500,000, 550,000, 600,000, 650,000, 700,000, 750,000, 800,000, 850,000, 900,000, 950,000, 1,000,000, 1,100,000, 1,200,000, or 1,250,000 M⁻¹cm⁻¹, and any range defined therebetween.

In some embodiments, a compound of the present invention has a fluorescence quantum yield at 405 nm in a range of 0.04 to 0.35, including 0.04, 0.10, 0.15, 0.20, 0.25, 0.30, 0.34, 0.35, and any range defined there between.

In some embodiments, a compound of the present invention has a second lowest (Qx) energy absorption band that is red-shifted (e.g., by at least 20 nm) relative to an excitation wavelength of the first porphyrin. In some embodiments, a compound of the invention is red shifted so that it does not interact or does not substantially interact with 488 nm laser emissions.

In some embodiments, a compound of the present invention has a peak emission wavelength and a peak excitation wavelength and the difference between the peak emission wavelength and peak excitation wavelength is at least 50 nm, and in some embodiments, at least 80 nm.

In some embodiments, a compound of the present invention has an emission wavelength from the first porphyrin that does not overlap with an emission wavelength from the first hydroporphyrin. In some embodiments, a compound of the present invention has an emission wavelength from the first porphyrin that does not overlap with the peak emission wavelength from the first hydroporphyrin.

Also provided according to embodiments of the invention is a composition that includes a compound (also referred to herein as the “active compound”) and/or a particle of the invention. In some embodiments, the composition includes water and the compound and/or the particle are present in water, optionally wherein the compound and/or particle has a solubility in water at room temperature in a range of about 1 mg/mL to about 10, 20, 30, 40, 50, 60, 70, 80, 90, or 100 mg/mL or more.

In some embodiments, the composition is devoid of an organic solvent. In some embodiments, a composition of the invention includes a first compound (e.g., a first luminescent compound) having a first absorption and emission spectra comprising a first emission wavelength and a second compound (e.g., a second luminescent compound) having a second absorption and emission spectra comprising a second emission wavelength, wherein the first and second emission wavelengths are different and/or distinct and the first and second 1 compounds are both compounds of the invention. In some embodiments, the first and second compounds are each excited by the same excitation wavelength. As such, in such embodiments, a single absorbance wavelength may be used to produce a variety of different emission wavelengths.

In some embodiments, additional chromophores and/or compounds (e.g., luminescent compounds) may be included in a composition of the invention. Examples of such compounds include, but are not limited to, perylenes, carotenoid, dipyrrinoatoborondifluoride, or bid(dipyrrinato)metal complexes. In some embodiments, such additional chromophores and/or compounds may enhance absorption in selected spectral regions.

Compounds of the present invention may be provided as pharmaceutically acceptable salts. Such salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethyl-benzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and other metal salts, such as but not limited to sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates and fumarates. Pharmaceutically acceptable esters include, but are not limited to, alkyl, alkenyl, alkynyl, aryl, heteroaryl, aralkyl, heteroaralkyl, cycloalkyl and heterocyclyl esters of acidic groups, including, but not limited to, carboxylic acids, phosphoric acids, phosphinic acids, sulfonic acids, sulfinic acids and boronic acids.

Active compounds of the present invention include prodrugs of the compounds described herein. As noted above, a “prodrug” is a compound that, upon in vivo administration, is metabolized by one or more steps or processes or otherwise converted to the biologically, pharmaceutically or therapeutically active form of the compound. To produce a prodrug, the pharmaceutically active compound is modified such that the active compound will be regenerated by metabolic processes. The prodrug may be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady (1985) Medicinal Chemistry A Biochemical Approach, Oxford University Press, New York, pages 388-392).

In some embodiments, a “neat” composition consisting of an active compound of the present invention or the pharmaceutically acceptable salts, prodrugs, or conjugates thereof (e.g., with a targeting agent such as a protein, peptide or antibody) may be provided.

In some embodiments, the present invention may provide compositions comprising or consisting essentially of an active compound of the present invention (or the pharmaceutically acceptable salts, prodrugs, or conjugates thereof (e.g., with a targeting agent such as a protein, peptide or antibody)) in a solvent. The amount of solvent is not critical and may comprise from about 0.01 or 1 to about 99 or 99.99 percent by weight of the composition. It will be appreciated that agitation may be required to break agglomerated particles back into solution prior to determining molar absorption, but that some level of agglomeration may actually be desired for practical use of the composition. Suitable solvents depend upon the particular compound and intended use for that compound, but include both organic solvents, aqueous solvents and combinations thereof.

The compositions, either in the “neat” form or mixed with a solvent, may have or exhibit a loss of not more than 10, 15 or 20 percent by weight of the compound of the present invention (due to degradation thereof) when stored in a sealed vessel (e.g., a flask ampoule or vial), at room temperature in the absence of ambient light for at least 3 or 4 months. Degradation can be determined by spectroscopy, thin-layer chromatography, NMR spectroscopy, and/or mass spectrometry, in accordance with known techniques.

According to some embodiments provided are pharmaceutical compositions. A pharmaceutical composition of the present invention may comprise a therapeutically effective amount of one or more of the compounds of the present invention, which may be useful in the prevention, treatment, and/or amelioration of one or more of the symptoms of diseases or disorders associated with hyperproliferating tissue or neovascularization, or in which hyperproliferating tissue or neovascularization is implicated, in a pharmaceutically acceptable carrier. Diseases or disorders associated with hyperproliferating tissue or neovascularization include, but are not limited to, cancer, psoriasis, atherosclerosis, heart disease, and age-related macular degeneration. Pharmaceutical carriers suitable for administration of the compounds provided herein include any such carriers known to those skilled in the art to be suitable for the particular mode of administration.

Pharmaceutical compositions may exhibit the absorption characteristics and/or storage and/or stability characteristics described herein.

In addition, the compounds may be formulated as the sole pharmaceutically active ingredient in the composition or may be combined with other active ingredients.

The compositions may contain one or more compounds of the present invention. In some embodiments, the compounds may be formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. In one embodiment, the compounds described above are formulated into pharmaceutical compositions using techniques and procedures well known in the art (see, e.g., Ansel, Introduction to Pharmaceutical Dosage Forms, Fourth Edition 1985, 126).

In the compositions, effective concentrations of one or more compounds or pharmaceutically acceptable derivatives thereof may be (are) mixed with a suitable pharmaceutical carrier. The compounds may be derivatized as the corresponding salts, esters, enol ethers or esters, acetals, ketals, orthoesters, hemiacetals, hemiketals, acids, bases, solvates, hydrates or prodrugs prior to formulation, as described above. The concentrations of the compounds in the compositions may be effective for delivery of an amount, upon administration, that treats, prevents, and/or ameliorates one or more of the symptoms of diseases or disorders associated with hyperproliferating tissue or neovascularization or in which hyperproliferating tissue or neovascularization is implicated.

In one embodiment, the compositions are formulated for single dosage administration. To formulate a composition, the weight fraction of a compound of the present invention is dissolved, suspended, dispersed, or otherwise mixed in a selected carrier at an effective concentration such that the treated condition is relieved, prevented, or one or more symptoms may be ameliorated.

The active compound may be included in the pharmaceutically acceptable carrier in an amount sufficient to exert a therapeutically useful effect in the absence of undesirable side effects on the subject treated. The therapeutically effective concentration may be determined empirically by testing the compounds in in vitro and in vivo systems described herein and in U.S. Pat. No. 5,952,366 to Pandey et al. (1999) and then extrapolated therefrom for dosages for humans.

The concentration of active compound in the pharmaceutical composition may depend on absorption, inactivation and excretion rates of the active compound, the physicochemical characteristics of the compound, the dosage schedule, and/or the amount administered as well as other factors known to those of skill in the art. For example, the amount that is delivered may be sufficient to ameliorate one or more of the symptoms of diseases or disorders associated with hyperproliferating tissue or neovascularization or in which hyperproliferating tissue or neovascularization is implicated, as described herein.

In one embodiment, a therapeutically effective dosage should produce a serum concentration of the active ingredient of from about 0.1 ng/ml to about 50-100 μg/ml. In one embodiment, a therapeutically effective dosage is from 0.001, 0.01 or 0.1 to 10, 100 or 1000 mg of active compound per kilogram of body weight per day. Pharmaceutical dosage unit forms may be prepared to provide from about 0.01 mg, 0.1 mg or 1 mg to about 500 mg, 1000 mg or 2000 mg, and in one embodiment from about 10 mg to about 500 mg of the active ingredient or a combination of essential ingredients per dosage unit form.

The active ingredient may be administered at once or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

In instances in which the compounds exhibit insufficient solubility, methods for solubilizing compounds may be used. Such methods are known to those of skill in this art, and include, but are not limited to, using cosolvents, such as dimethylsulfoxide (DMSO), using surfactants, such as TWEEN™, or dissolution in aqueous sodium bicarbonate. Derivatives of the compounds, such as prodrugs of the compounds may also be used in formulating effective pharmaceutical compositions.

Upon mixing or addition of the compound(s), the resulting mixture may be a solution, suspension, emulsion or the like. The form of the resulting mixture depends upon a number of factors, including the intended mode of administration and the solubility of the compound in the selected carrier or vehicle. The effective concentration may be sufficient for ameliorating the symptoms of the disease, disorder or condition treated and may be empirically determined.

The pharmaceutical compositions may be provided for administration to humans and/or animals in unit dosage forms, such as tablets, capsules, pills, powders, granules, sterile parenteral solutions or suspensions, and oral solutions or suspensions, and oil-water emulsions containing suitable quantities of the compounds or pharmaceutically acceptable derivatives thereof. The pharmaceutically therapeutically active compounds and derivatives thereof are, in one embodiment, formulated and administered in unit-dosage forms or multiple-dosage forms. Unit-dose forms as used herein refers to physically discrete units suitable for human and animal subjects and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampoules and syringes and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

Liquid pharmaceutically administrable compositions may, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, solubilizing agents, pH buffering agents and the like, for example, acetate, sodium citrate, cyclodextrin derivatives, sorbitan monolaurate, triethanolamine sodium acetate, triethanolamine oleate, and other such agents.

Actual methods of preparing such dosage forms are known, or will be apparent, to those skilled in this art; for example, see Remington's Pharmaceutical Sciences, Mack Publishing Company, Easton, Pa., 15th Edition, 1975.

Dosage forms or compositions containing active ingredient in the range of 0.005% to 100% with the balance made up from non-toxic carrier may be prepared. Methods for preparation of these compositions are known to those skilled in the art. The contemplated compositions may contain 0.001%-100% active ingredient, in one embodiment 0.1-95%, in another embodiment 75-85%.

In some embodiments, a composition of the present invention may be suitable for oral administration. Oral pharmaceutical dosage forms are either solid, gel or liquid. The solid dosage forms are tablets, capsules, granules, and bulk powders. Types of oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art.

In certain embodiments, the formulations are solid dosage forms, in one embodiment, capsules or tablets. The tablets, pills, capsules, troches and the like may contain one or more of the following ingredients, or compounds of a similar nature: a binder; a lubricant; a diluent; a glidant; a disintegrating agent; a coloring agent; a sweetening agent; a flavoring agent; a wetting agent; an emetic coating; and a film coating. Examples of binders include microcrystalline cellulose, gum tragacanth, glucose solution, acacia mucilage, gelatin solution, molasses, polvinylpyrrolidine, povidone, crospovidones, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof, and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic-coatings include fatty acids, fats, waxes, shellac, ammoniated shellac and cellulose acetate phthalates. Film coatings include hydroxyethylcellulose, gellan gum, sodium carboxymethylcellulose, polyethylene glycol 4000 and cellulose acetate phthalate.

The compound, or pharmaceutically acceptable derivative thereof, may be provided in a composition that protects it from the acidic environment of the stomach. For example, the composition may be formulated in an enteric coating that maintains its integrity in the stomach and releases the active compound in the intestine. The composition may also be formulated in combination with an antacid or other such ingredient. When the dosage unit form is a capsule, it may contain, in addition to material of the above type, a liquid carrier such as a fatty oil. In addition, dosage unit forms may contain various other materials which modify the physical form of the dosage unit, for example, coatings of sugar and other enteric agents. The compounds may be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

The active materials may also be mixed with other active materials which do not impair the desired action, or with materials that supplement the desired action, such as antacids, H2 blockers, and diuretics. The active ingredient is a compound or pharmaceutically acceptable derivative thereof as described herein. Higher concentrations, up to about 98% by weight of the active ingredient may be included.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wefting agents. Pharmaceutically acceptable substances used in effervescent granules, to be reconstituted into a liquid oral dosage form, include organic acids and a source of carbon dioxide. Coloring and flavoring agents are used in all of the above dosage forms. Solvents include glycerin, sorbitol, ethyl alcohol and syrup. Examples of preservatives include glycerin, methyl and propylparaben, benzoic acid, sodium benzoate and alcohol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, xanthan gum, Veegum and acacia. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic acids include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation. For a solid dosage form, the solution or suspension, in for example propylene carbonate, vegetable oils or triglycerides, is in one embodiment encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi-solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include those set forth in U.S. Pat. Nos. RE28,819 and 4,358,603. Briefly, such formulations include, but are not limited to, those containing a compound provided herein, a dialkylated mono- or poly-alkylene glycol, including, but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(loweralkyl) acetals of loweralkyl aldehydes such as acetaldehyde diethyl acetal.

Parenteral administration, in one embodiment characterized by injection, either subcutaneously, intramuscularly or intravenously is also contemplated herein. Injectables may be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Implantation of a slow-release or sustained-release system, such that a constant level of dosage is maintained (see, e.g., U.S. Pat. No. 3,710,795) is also contemplated herein. Briefly, a compound provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, xanthan gum, hydroxypropyl methylcellulose and polyvinylpyrrolidone. Emulsifying agents include Polysorbate 80 (TWEEN™ 80). A sequestering or chelating agent of metal ions includes EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

The concentration of the pharmaceutically active compound is adjusted so that an injection provides an effective amount to produce the desired pharmacological effect. The exact dose depends on the age, weight and condition of the subject or animal as is known in the art.

The unit-dose parenteral preparations are packaged in an ampoule, a vial or a syringe with a needle. All preparations for parenteral administration must be sterile, as is known and practiced in the art.

Illustratively, intravenous or intraarterial infusion of a sterile aqueous solution containing an active compound is an effective mode of administration. Another embodiment is a sterile aqueous or oily solution or suspension containing an active material injected as necessary to produce the desired pharmacological effect.

Injectables are designed for local and systemic administration. In one embodiment, a therapeutically effective dosage is formulated to contain a concentration of at least about 0.1% w/w up to about 90% w/w or more, in certain embodiments more than 1% w/w of the active compound to the treated tissue(s).

The compound may be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

Lyophilized powders, which can be reconstituted for administration as solutions, emulsions and other mixtures, may also be used to carry out the present invention. They may also be reconstituted and formulated as solids or gels.

The sterile, lyophilized powder is prepared by dissolving a compound provided herein, or a pharmaceutically acceptable derivative thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides the desired formulation. In one embodiment, the resulting solution will be apportioned into vials for lyophilization. Each vial will contain a single dosage or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carrier. The precise amount depends upon the selected compound. Such amount can be empirically determined.

Topical mixtures may be prepared as described for the local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds or pharmaceutically acceptable derivatives thereof may be formulated as aerosols for topical application, such as by inhalation (see, e.g., U.S. Pat. Nos. 4,044,126; 4,414,209; and 4,364,923, which describe aerosols for delivery of a steroid useful for treatment of inflammatory diseases, particularly asthma). These formulations for administration to the respiratory tract may be in the form of an aerosol or solution for a nebulizer, or as a microfine powder for insufflation, alone or in combination with an inert carrier such as lactose. In such a case, the particles of the formulation will, in one embodiment, have diameters of less than 50 microns, in one embodiment less than 10 microns.

The compounds may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients may be administered. These solutions, particularly those intended for ophthalmic use, may be formulated as 0.01%-10% isotonic solutions, pH about 5-7, with appropriate salts.

Other routes of administration, such as transdermal patches, including iontophoretic and electrophoretic devices, and rectal administration, are also contemplated herein.

Transdermal patches, including iontophoretic and electrophoretic devices, are well known to those of skill in the art. For example, such patches are disclosed in U.S. Pat. Nos. 6,267,983; 6,261,595; 6,256,533; 6,167,301; 6,024,975; 6,010715; 5,985,317; 5,983,134; 5,948,433 and 5,860,957.

For example, pharmaceutical dosage forms for rectal administration are rectal suppositories, capsules and tablets for systemic effect. Rectal suppositories as used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The weight of a rectal suppository, in one embodiment, is about 2 to 3 gm.

Tablets and capsules for rectal administration are manufactured using the same pharmaceutically acceptable substance and by the same methods as for formulations for oral administration.

The compounds provided herein, or pharmaceutically acceptable derivatives thereof, may also be formulated to be targeted to a particular tissue, receptor, infecting agent or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652; 6,274,552; 6,271,359; 6,253,872; 6,139,865; 6,131,570; 6,120,751; 6,071,495; 6,060,082; 6,048,736; 6,039,975; 6,004,534; 5,985,307; 5,972,366; 5,900,252; 5,840,674; 5,759,542 and 5,709,874.

In one embodiment, liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS. In some embodiments of the invention, provided is a use of a compound, particle, composition, and/or kit of the invention in flow cytometry. Flow cytometry is known and described in, for example, U.S. Pat. Nos. 5,915,925; 6,248,590; 6,589,792; 6,890,487, 8,980,565, and 9,417,245. In some embodiments, a target (e.g., compound, particle, cell, etc.) is labelled with a compound of the invention and the labelled target is subsequently detected such as in a flow cytometry method. Labelling can be carried out by any suitable technique such as coupling the compound of the invention to another compound such as an antibody which in turn specifically binds to the particle or cell, by uptake or internalization of the compound into the cell or particle, by non-specific adsorption of the compound to the cell or particle, etc. A compound described herein may be useful in flow cytometry, and flow cytometry techniques (including fluorescent activated cell sorting or FACS) may be carried out in accordance with known techniques or variations thereof which will be apparent to those skilled in the art based upon the instant disclosure.

In some embodiments, a method of the present invention comprises one, two, or more (e.g., 1, 2, 3, 4, 5, 6, 7, or more) labelled targets. A “labelled target” as used herein refers to a target (e.g., a compound, particle, cell, etc.) that is associated with (e.g., bound to such as covalently bound to or non-covalently bound to) a detectable compound. The detectable compound may be excited at an excitation wavelength band and emit light at an emission wavelength band. In some embodiments, a labelled target and/or detectable compound may comprise a compound of the present invention. In some embodiments, a method of the present invention comprises a first labelled target and a second labelled target, wherein the first labelled target comprises a compound of the present invention and the second labelled target is different than the first labelled target. The first labelled target and the second labelled target each comprise a detectable compound that is excited at an excitation wavelength band (optionally excited at the same excitation wavelength or a different excitation wavelength) and the first labelled target may have a different emission wavelength band than an emission wavelength band of the second labeled target. In some embodiments, emission wavelength bands of the first and second labelled targets are characterized by peaks that are separated from one another by at least 5 nanometers (i.e., the first labelled target has a peak emission wavelength band that is at least 5 nanometers away from the peak emission wavelength band of the second labelled target). The method may comprise detecting the first labelled target, detecting the second labelled target, and distinguishing the first labelled target and the second labelled target from each other, optionally wherein the distinguishing is carried out by detecting and/or determining an emission wavelength band associated with the first labelled target and/or detecting and/or determining an emission wavelength band associated with the second labelled target. In some embodiments, the first labelled target comprises a first compound of the present invention and the second labelled target comprises a second compound of the present invention, wherein the first and second compounds have a different emission wavelength band (e.g., the peak emission wavelength band of the first compound is different than (e.g., at least 5 nanometers away from) the peak emission wavelength band of the second compound). In some embodiments, the first labelled target comprises a first compound of the present invention and the second labelled target comprises detectable compound that is not a compound of the present invention (e.g., that is not a dyad of the present invention), optionally wherein the detectable compound of the second labelled target is a chlorin, bacteriochlorin, isobacteriochlorin, or porphyrin.

Also provided herein are methods for using compounds, compositions, particles, and kits of the invention. In some embodiments, provided are methods of detecting cells and/or particles using flow cytometry. In some embodiments, a method includes labeling cells and/or particles with a compound, particle, composition, and/or kit of the invention; and detecting the compound by flow cytometry, thereby detecting the cells and/or particles.

For multicolor applications, members of a set of porphyrinic pigments can be tuned to absorb/emit at different wavelengths through use of auxochromes. In particular, utilizing the same porphyrin donor but different hydroporphyrin acceptors in a set may allow all members to be excited at the same absorption wavelength but provide a gradation of emission wavelengths, all with large absorption fluorescence spacings. In typical multicolor applications, a set of antibodies is labeled with a set of fluorophores (one type of fluorophore for a given monoclonal antibody). The ability to discriminate multicolors would enable the set of antibodies to be employed in parallel against a heterogeneous pool of cells. The present invention provides for spectrally distinct, stable fluorophores. Alternatively, a series of spectrally distinct donor porphyrins can be employed in conjunction with a single type of acceptor. In this case, one relies on a single wavelength of detection but different wavelengths of excitation. Regardless of experimental design, the spectral tuning can be achieved through use of (i) different pigments, (ii) different metals in porphyrinic pigments, and (iii) use of auxochromes.

Also provided are methods of detecting a tissue and/or agent (e.g., a cell, infecting agent, etc.) in a subject. In some embodiments, the method includes administering to the subject a compound, a particle, a composition, or kit of the invention, optionally wherein the compound associates with the tissue and/or agent; and detecting the compound within the subject, thereby detecting the tissue and/or agent. For optical imaging, in some embodiments, two porphyrinic chromophores can be linked with a hydroporphyrin to form a dyad possessing a large shift (e.g., >50 nm) between the absorption and fluorescence emission maxima. The large spectral spacing minimizes artifacts (due to scattered excitation light reaching the fluorescence detection system) that compromise imaging quality, especially in deep tissue applications. The excitation-light rejection efficiency may increase with increasing absorption-fluorescence spacing. In some embodiments, compounds that include bacteriochlorins as the acceptor enable excitation at the maximum of the relative sharp (<20 nm) and intense NIR band of the energy absorber/donor subunit and detection at the NIR peak of the emitter/acceptor. In some embodiments, spectral features of bacteriochlorins are shifted (e.g., over at least 50 nm) to yield donors and acceptors that may be useful for optical imaging.

Also provided are methods of treating a cell and/or tissue (e.g., a diseased cell and/or tissue) in a subject in need thereof. In some embodiments, a method includes administering a compound, a particle, a composition, or kit of the invention, optionally wherein the compound associates with the cell and/or tissue and irradiating the subject or a portion thereof (e.g., a location where the cell and/or tissue are present) with light of a wavelength and intensity sufficient to treat the cell and/or tissue, optionally wherein the light activates the compound or a part thereof. In some embodiments, the cell and/or tissue is a hyperproliferative tissue (e.g., a tumor).

In some embodiments of the invention, provided is a use of a compound, particle, composition, and/or kit of the invention in imaging (e.g., photoacoustic imaging) and/or microscopy.

Also provided herein are methods of imaging a tissue and/or agent (e.g., a cell, infecting agent, etc.) in a subject. In some embodiments, a method includes administering to the subject a compound, particle, composition, and/or kit of the invention; and detecting the compound within the subject, thereby imaging the tissue and/or agent. In particular embodiments, detecting the compound within the subject includes irradiating the subject or a portion thereof (e.g., a location where the compound is present and/or a location to be imaged) with light of a wavelength and intensity sufficient to produce an ultrasonic wave (e.g., an ultrasonic pressure wave), optionally wherein the irradiating is performed using a laser and/or by exposing the subject to one or more non-ionizing laser pulse(s). In some embodiments, detecting the compound within the subject includes detecting an ultrasound wave, optionally using an ultrasound detector. In some embodiments, the method of imaging the tissue and/or agent in the subject comprises photoacoustic imaging of the tissue and/or agent.

In some embodiments, compounds of the invention may be used in photodynamic therapy applications. In this application, it will be the acceptor chromophore will be tuned by choice of central metal and peripheral substituents to have (1) a high yield of formation of the excited triplet state from the excited singlet state, (2) long triplet excited-state lifetime, and (3) high yield of energy transfer from the triplet excited state to oxygen to form the reactive oxygen species (ROS), namely its lowest singlet delta excited state. In some embodiments, to enhance the first factor, a different metal ion (a heavy one such as palladium) may be utilized in the acceptor chromophore to enhance spin orbit coupling and thus the rate of singlet-triplet intersystem crossing. Thus, fluorescence may be virtually eliminated, but that is not essential for this application. However, it is noteworthy that the tetrapyrrole chromophores generally have high triplet excited state yields even in the absence of such a heavy atom effect. Thus, one may be able to utilize the same acceptor chromophore for both detecting its presence in the proper location (cancer cell, membrane compartment, etc.) by optical imaging at low light intensity and then for photodynamic therapy at high light intensity. This may benefit the ability to assess both the localization and effect of the photodynamic therapy reagent. These applications will be facilitated by the use of the compounds of the invention over monomeric reagents due to the ability to independently tune absorption properties (e.g., of the porphyrin) and the reactive properties of the acceptor (e.g., hydroporphyrin) essential for cell killing.

In another embodiment, the disclosed compounds may be targeted to specific target tissues or target compositions using ligands specific for the target tissue or target composition, for example, using ligands or ligand-receptor pairs such as antibodies and antigens. Antibodies against tumor antigens and against pathogens are known. For example, antibodies and antibody fragments which specifically bind markers produced by or associated with tumors or infectious lesions, including viral, bacterial, fungal and parasitic infections, and antigens and products associated with such microorganisms have been disclosed, inter alia, in Hansen et al., U.S. Pat. No. 3,927,193 and Goldenberg, U.S. Pat. Nos. 4,331,647; 4,348,376; 4,361,544; 4,468,457; 4,444,744; 4,818,709 and 4,624,846. Antibodies against an antigen, e.g., a gastrointestinal, lung, breast, prostate, ovarian, testicular, brain or lymphatic tumor, a sarcoma or a melanoma, may be used.

A wide variety of monoclonal antibodies against infectious disease agents have been developed, and are summarized in a review by Polin, in Eur. J. Clin. Microbiol., 3(5): 387-398 (1984), showing ready availability. These include monoclonal antibodies (MAbs) against pathogens and their antigens such as the following: Anti-bacterial Mabs such as those against Streptococcus agalactiae, Legionella pneumophilia, Streptococcus pyogenes, Esherichia coli, Neisseria gonorrhosae, Neisseria meningitidis, Pneumococcus, Hemophilis influenzae B, Treponema pallidum, Lyme disease, spirochetes, Pseudomonas aeruginosa, Mycobacterium leprae, Brucella abortus, Mycobacterium tuberculosis, Tetanus toxin, Anti-protozoan Mabs such as those against Plasmodium falciparum, Plasmodium vivax, Toxoplasma gondii, Trypanosoma rangeli, Trypanosoma cruzi, Trypanosoma rhodesiensei, Trypanosoma brucei, Schistosoma mansoni, Schistosoma japanicum, Mesocestoides corti, Emeria tenella, Onchocerca volvulus, Leishmania tropica, Trichinella spiralis, Theileria parva, Taenia hydatigena, Taenia ovis, Taenia saginata, Anti-viral MAbs such as those against HIV-1, -2, and -3, Hepatitis A, B, C, D, Rabies virus, Influenza virus, Cytomegalovirus, Herpes simplex I and II, Human serum parvo-like virus, Respiratory syncytial virus, Varicella-Zoster virus, Hepatitis B virus, Measles virus, Adenovirus, Human T-cell leukemia viruses, Epstein-Barr virus, Mumps virus, Sindbis virus, Mouse mammary tumor virus, Feline leukemia virus, Lymphocytic choriomeningitis virus, Wart virus, Blue tongue virus, Sendai virus, Reo virus, Polio virus, Dengue virus, Rubella virus, Murine leukemia virus, Antimycoplasmal MAbs such as those against Acholeplasma laidlawii, Mycoplasma arthritidis, M. hyorhinis, M. orale, M. arginini, M. pneumonia; etc.

Suitable MAbs have been developed against most of the micro-organisms (bacteria, viruses, protozoa, other parasites) responsible for the majority of infections in humans, and many have been used previously for in vitro diagnostic purposes. These antibodies, and newer MAbs that can be generated by conventional methods, may be appropriate for use as target agents with the compounds provided herein.

MAbs against malaria parasites can be directed against the sporozoite, merozoite, schizont and gametocyte stages. Monoclonal antibodies have been generated against sporozoites (circumsporozoite antigen), and have been shown to neutralize sporozoites in vitro and in rodents (N. Yoshida et al., Science 207: 71-73 (1980)). Monoclonal antibodies to T. gondii, the protozoan parasite involved in toxoplasmosis have been developed (Kasper et al., J. Immunol. 129: 1694-1699 (1982). MAbs have been developed against schistosomular surface antigens and have been found to act against schistosomulae in vivo or in vitro (Simpson et al., Parasitology 83: 163-177 (1981); Smith et al., Parasitology 84: 83-91 (1982); Gryzch et al., J. Immunol. 129: 2739-2743 (1982); Zodda et al., J. Immunol. 129: 2326-2328 (1982); Dissous et al., J. Immunol. 129: 2232-2234 (1982).

It should be noted that mixtures of antibodies and immunoglobulin classes may be used, as may hybrid antibodies. Multispecific, including bispecific and hybrid, antibodies and antibody fragments may be used in the methods of the present invention for detecting and treating target tissue and may comprise at least two different substantially monospecific antibodies or antibody fragments, wherein at least two of the antibodies or antibody fragments specifically bind to at least two different antigens produced or associated with the targeted lesion or at least two different epitopes or molecules of a marker substance produced or associated with the target tissue. Multispecific antibodies and antibody fragments with dual specificities can be prepared analogously to the anti-tumor marker hybrids disclosed in U.S. Pat. No. 4,361,544. Other techniques for preparing hybrid antibodies are disclosed in, e.g., U.S. Pat. Nos. 4,474,893 and 4,479,895, and in Milstein et al., Immunol. Today 5: 299 (1984).

Antibody fragments useful in the present invention include F(ab′)₂, F(ab)₂, Fab′, Fab, Fv and the like including hybrid fragments. In some embodiments, fragments are Fab′, F(ab′)₂, Fab, and F(ab)₂. Also useful are any subfragments retaining the hypervariable, antigen-binding region of an immunoglobulin and having a size similar to or smaller than a Fab′ fragment. This will include genetically engineered and/or recombinant proteins, whether single-chain or multiple-chain, which incorporate an antigen-binding site and otherwise function in vivo as targeting vehicles in substantially the same way as natural immunoglobulin fragments. Such single-chain binding molecules are disclosed in U.S. Pat. No. 4,946,778, which is hereby incorporated by reference. Fab′ antibody fragments may be conveniently made by reductive cleavage of F(ab′)₂ fragments, which themselves may be made by pepsin digestion of intact immunoglobulin. Fab antibody fragments may be made by papain digestion of intact immunoglobulin, under reducing conditions, or by cleavage of F(ab)₂ fragments which result from careful papain digestion of whole immunoglobulin.

A ligand or one member of a ligand-receptor binding pair may be conjugated to the compounds provided herein for targeting the compounds to specific target tissues or target compositions. Examples of ligand-receptor binding pairs are set out in U.S. Pat. Nos. 4,374,925 and 3,817,837, the teachings of which are incorporated herein by reference.

Many compounds that can serve as targets for ligand-receptor binding pairs, and more specifically, antibodies, have been identified, and the techniques to construct conjugates of such ligands with photosensitizers are well known to those of ordinary skill in this art. For example, Rakestraw et al. teaches conjugating Sn(IV) chlorine6 via covalent bonds to monoclonal antibodies using a modified dextran carrier (Rakestraw, S. L., Tompkins, R. D., and Yarmush, M. L., Proc. Nad. Acad. Sci. USA 87: 4217-4221 (1990). The compounds disclosed herein may also be conjugated to a ligand, such as an antibody, by using a coupling agent. Any bond which is capable of linking the components such that they are stable under physiological conditions for the time needed for administration and treatment is suitable. In some embodiments, the bond may be a covalent linkage. The link between two components may be direct, e.g., where a photosensitizer is linked directly to a targeting agent, or indirect, e.g., where a photosensitizer is linked to an intermediate and that intermediate being linked to the targeting agent.

A coupling agent should function under conditions of temperature, pH, salt, solvent system, and other reactants that substantially retain the chemical stability of the photosensitizer, the backbone (if present), and the targeting agent. Coupling agents should link component moieties stably, but such that there is only minimal or no denaturation or deactivation of the photosensitizer or the targeting agent. Many coupling agents react with an amine and a carboxylate, to form an amide, or an alcohol and a carboxylate to form an ester. Coupling agents are known in the art (see, e.g., M. Bodansky, “Principles of Peptide Synthesis”, 2nd ed., and T. Greene and P. Wuts, “Protective Groups in Organic Synthesis,” 2nd Ed, 1991, John Wiley, NY).

The conjugates of the compounds provided herein with ligands such as antibodies may be prepared by coupling the compound to targeting moieties by cleaving the ester on the “E” ring and coupling the compound via peptide linkages to the antibody through an N terminus, or by other methods known in the art. A variety of coupling agents, including cross-linking agents, may be used for covalent conjugation. Examples of cross-linking agents include N,N′-dicyclohexylcarbodiimide (DCC), N-succinimidyl-S-acetyl-thioacetate (SATA), N-succinimidyl-3-(2-pyridyidi-thio)propionate (SPDP), ortho-phenylene-dimaleimide (o-PDM), and sulfosuccinimidyl 4-(N-maleimido-methyl)-cyclohexane-1-carboxylate (sulfo-SMCC). See, e.g., Karpovsky et al., J. Exp. Med. 160:1686 (1984); and Liu, M A et al., Proc. Natl. Acad. Sci. USA 82: 8648 (1985). Other methods include those described by Brennan et al., Science 229: 81-83 (1985) and Glennie et al., J. Immunol. 139: 2367-2375 (1987). A large number of coupling agents for peptides and proteins, along with buffers, solvents, and methods of use, are described in the Pierce Chemical Co. catalog, pages 0-90 to 0-110 (1995, Pierce Chemical Co., 3747 N. Meridian Rd., Rockford Ill., 61105, U.S.A.), which catalog is hereby incorporated by reference.

For example, DCC is a useful coupling agent that may be used to promote coupling of the alcohol NHS to chlorin e6 in DMSO forming an activated ester which may be cross-linked to polylysine. DCC is a carboxy-reactive cross-linker commonly used as a coupling agent in peptide synthesis, and has a molecular weight of 206.32. Another useful cross-linking agent is SPDP, a heterobifunctional cross-linker for use with primary amines and sulfhydryl groups. SPDP has a molecular weight of 312.4, a spacer arm length of 6.8 angstroms, is reactive to NHS-esters and pyridyldithio groups, and produces cleavable cross-linking such that, upon further reaction, the agent is eliminated so the photosensitizer may be linked directly to a backbone or targeting agent. Other useful conjugating agents are SATA for introduction of blocked SH groups for two-step cross-linking, which is deblocked with hydroxylamine-HCl, and sulfo-SMCC, reactive towards amines and sulfhydryls. Other cross-linking and coupling agents are also available from Pierce Chemical Co. Additional compounds and processes, particularly those involving a Schiff base as an intermediate, for conjugation of proteins to other proteins or to other compositions, for example to reporter groups or to chelators for metal ion labeling of a protein, are disclosed in EPO 243,929 A2 (published Nov. 4, 1987).

Photosensitizers which contain carboxyl groups may be joined to lysine F-amino groups in the target polypeptides either by preformed reactive esters (such as N-hydroxy succinimide ester) or esters conjugated in situ by a carbodiimide-mediated reaction. The same applies to photosensitizers which contain sulfonic acid groups, which may be transformed to sulfonyl chlorides which react with amino groups. Photosensitizers which have carboxyl groups may be joined to amino groups on the polypeptide by an in situ carbodiimide method. Photosensitizers may also be attached to hydroxyl groups, of serine or threonine residues or to sulfhydryl groups of cysteine residues.

Methods of joining components of a conjugate, e.g., coupling polyamino acid chains bearing photosensitizers to antibacterial polypeptides, may use heterobifunctional cross linking reagents. These agents bind a functional group in one chain and to a different functional group in the second chain. These functional groups typically are amino, carboxyl, sulfhydryl, and aldehyde. There are many permutations of appropriate moieties which will react with these groups and with differently formulated structures, to conjugate them together. See the Pierce Catalog, and Merrifield, R. B. et al., Ciba Found Symp. 186: 5-20 (1994).

The compounds or pharmaceutically acceptable derivatives thereof may be packaged as articles of manufacture containing packaging material, a compound or pharmaceutically acceptable derivative thereof provided herein, which is effective for modulating the activity of hyperproliferating tissue or neovascularization, or for treatment, prevention or amelioration of one or more symptoms of hyperproliferating tissue or neovascularization mediated diseases or disorders, or diseases or disorders in which hyperproliferating tissue or neovascularization activity, is implicated, within the packaging material, and a label that indicates that the compound or composition, or pharmaceutically acceptable derivative thereof, is used for modulating the activity of hyperproliferating tissue or neovascularization, or for treatment, prevention or amelioration of one or more symptoms of hyperproliferating tissue or neovascularization mediated diseases or disorders, or diseases or disorders in which hyperproliferating tissue or neovascularization is implicated.

The articles of manufacture provided herein contain packaging materials. Packaging materials for use in packaging pharmaceutical products are well known to those of skill in the art. See, e.g., U.S. Pat. Nos. 5,323,907; 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the compounds and compositions provided herein are contemplated as are a variety of treatments for any disease or disorder in which hyperproliferating tissue or neovascularization is implicated as a mediator or contributor to the symptoms or cause.

In some embodiments, a compound of the present invention may be a photosensitizing compound. A photosensitizing compound may be administered to a subject before a target tissue, target composition and/or subject is subjected to illumination. The photosensitizing compound may be administered as described elsewhere herein.

The dose of the photosensitizing compound may be determined clinically. Depending on the photosensitizing compound used, an equivalent optimal therapeutic level may need to be established. A certain length of time may be allowed to pass for the circulating or locally delivered photosensitizer to be taken up by the target tissue. The unbound photosensitizer is cleared from the circulation during this waiting period, or additional time may optionally be provided for clearing of the unbound compound from non-target tissue. The waiting period may be determined clinically and may vary from compound to compound.

At the conclusion of this waiting period, a laser light source or a non-laser light source (including but not limited to artificial light sources such as fluorescent or incandescent light, or natural light sources such as ambient sunlight) may be used to activate the bound photosensitizer. The area of illumination may be determined by the location and/or dimension of the pathologic region to be detected, diagnosed or treated. The duration of illumination period may depend on whether detection or treatment is being performed, and may be determined empirically. A total or cumulative period of time anywhere from between about 4 minutes and 72 hours may be used. In one embodiment, the illumination period may be between about 60 minutes and 148 hours. In another embodiment, the illumination period may be between about 2 hours and 24 hours.

In some embodiments, the total fluence or energy of the light used for irradiating, as measured in Joules, may be between about 10 Joules and about 25,000 Joules; in some embodiments, between about 100 Joules and about 20,000 Joules; and in some embodiments, between about 500 Joules and about 10,000 Joules. Light of a wavelength and fluence sufficient to produce the desired effect may be selected, whether for detection by luminescence (e.g., fluorescence or phosphorescence) or for therapeutic treatment to destroy or impair a target tissue or target composition. Light having a wavelength corresponding at least in part with the characteristic light absorption wavelength of the photosensitizing agent may be used for irradiating the target issue.

The intensity or power of the light used may be measured in watts, with each Joule equal to one watt-sec. Therefore, the intensity of the light used for irradiating in the present invention may be substantially less than 500 mW/cm². Since the total fluence or amount of energy of the light in Joules is divided by the duration of total exposure time in seconds, the longer the amount of time the target is exposed to the irradiation, the greater the amount of total energy or fluence may be used without increasing the amount of the intensity of the light used. The present invention employs an amount of total fluence of irradiation that is sufficiently high to activate the photosensitizing agent.

In one embodiment of using compounds disclosed herein for photodynamic therapy, the compounds are injected into the mammal, e.g. human, to be diagnosed or treated. The level of injection may be between about 0.1 and about 0.5 umol/kg of body weight. In the case of treatment, the area to be treated is exposed to light at the desired wavelength and energy, e.g. from about 10 to 200 J/cm². In the case of detection, luminescence is determined upon exposure to light at a wavelength sufficient to cause the compound to fluoresce and/or phosphoresce at a wavelength different than that used to illuminate the compound. The energy used in detection is sufficient to cause fluorescence and/or phosphorescenece and is usually significantly lower than is required for treatment.

Also provided according to embodiments of the invention is a kit that includes a composition, compound and/or particle of the invention. In some embodiments, the kit includes one or more (or all) all compositions that are devoid of organic solvent. In some embodiments, a kit of the invention includes a first compound having a first absorption and emission spectra comprising a first emission wavelength and a second compound a second absorption and emission spectra comprising a second emission wavelength, wherein the first and second emission wavelengths are different and/or distinct and the first and second compounds are both a compound of the invention. In some embodiments, the first and second compounds are each excited by the same excitation wavelength.

Any one of the photosensitizing compounds disclosed herein or a pharmaceutically acceptable derivative thereof may be supplied in a kit along with instructions on conducting any of the methods disclosed herein. Instructions may be in any tangible form, such as printed paper, a computer disk that instructs a person how to conduct the method, a video cassette containing instructions on how to conduct the method, or computer memory that receives data from a remote location and illustrates or otherwise provides the instructions to a person (such as over the Internet). A person may be instructed in how to use the kit using any of the instructions above or by receiving instructions in a classroom or in the course of treating a subject using any of the methods disclosed herein, for example.

In some embodiments, provided is a method for using a compound of the present invention in photodynamic therapy (PDT) and/or photodynamic inactivation (PDI). Additional examples and specific examples of methods of using compounds and compositions of the present invention include, but are not limited to, the following:
(i) Treatment of opportunistic infections. Compounds, compositions and methods of the invention may be useful for PDT of opportunistic infections, particularly of soft tissue. For antimicrobial treatment (via PDT) of infections, particularly wound infections, the infecting organism can include (as non limiting examples) Staphylococcus aureus, Pseudomonas aeruginosa, and Escherichia coli. In nosocomial infections, P. aeruginosa is responsible for 8% of surgical-wound infections and 10% of bloodstream infections. In some embodiments the subjects are immunocompromised subjects, such as those afflicted with AIDS or undergoing treatment with immunosupressive agents.
(ii) Treatment of burns. Infections by S. aureus and gram-positive bacteria in general are particularly pronounced in burns (Lambrechts, 2005). The multidrug resistance of S. aureus presents significant medical challenges. In this regard, compounds, compositions and methods of the invention may be useful for the treatment of opportunistic infections of burns.
(iii) Sepsis. Compounds, compositions and methods of the invention may be useful for the PDT treatment of subjects afflicted with opportunistic infections of Vibrio vulnificus. V. vulnificus, a gram-negative bacterium, causes primary sepsis, wound infections, and gastrointestinal illness in humans.
(iv) Ulcers. Compounds, compositions and methods of the invention may be useful for PDT treatment of the bacterium that causes ulcers (Helicobacter pylori). In the clinic, treatment may be affected in any suitable manner, such as by insertion of a fiber optic cable (akin to an endoscope but with provisions for delivery of red or near-IR light) into the stomach or afflicted region.
(v) Periodontal disease. Compounds, compositions and methods of the invention may be useful in PDT for the treatment of periodontal disease, including gingivitis. Periodontal disease is caused by the overgrowth of bacteria, such as the gram-negative anaerobe Porphyromonas gingivalis. As with many PDT treatments, targeting or solubilizing entities in conjunction with the photoactive species are essential for appropriate delivery of the photoactive species to the desired cells. The oral pathogens of interest for targeting include Porphyromonas gingivalis, Actinobacillus actinomycetemcomitans, Bacteroides forsythus, Campylobacter rectus, Eikenella corrodens, Fusobacterium nucleatum subsp. Polymorphum, Actinomyces viscosus, and the streptococci. For such applications the compounds or compositions of the present invention may be topically applied (e.g., as a mouthwash or rinse) and then light administered with an external device, in-the-mouth instrument, or combination thereof.
(vi) Atherosclerosis. Compounds, compositions and methods of the invention may be useful in PDT to treat vulnerable atherosclerotic plaque. Without wishing to be bound to any particular theory, invading inflammatory macrophages are believed to secrete metalloproteinases that degrade a thin layer of collagen in the coronary arteries, resulting in thrombosis, which often is lethal (Demidova and Hamblin, 2004). Bacteriochlorins targeted to such inflammatory macrophages may be useful for PDT of vulnerable plaque.
(vii) Cosmetic and dermatologic applications. Compounds, compositions and methods of the invention may be useful in PDT to treat a wide range of cosmetic dermatological problems, such as hair removal, treatment of psoriasis, or removal of skin discoloration. Ruby lasers are currently used for hair removal; in many laser treatments melanin is the photosensitized chromophore. Such treatments work reasonably well for fair-skinned individuals with dark hair. Compounds, compositions and methods of the invention may be used as near-IR sensitizers for hair removal, which enables targeting a chromophore with a more specific and sharp absorption band.
(viii) Acne. Compounds, compositions and methods of the invention may be useful in PDT to treat acne. Acne vulgaris is caused by Propionibacterium acnes, which infects the sebaceous gland; some 80% of young people are affected. Here again, the growing resistance of bacteria to antibiotic treatment is leading to an upsurge of acne that is difficult to treat. Current PDT treatments of acne typically rely on the addition of aminolevulinic acid, which in the hair follicle or sebaceous gland is converted to free base porphyrins. Compounds and compositions of the invention may be administered to subjects topically or parenterally (e.g., by subcutaneous injection) depending upon the particular condition.
(ix) Infectious diseases. Compounds, compositions and methods of the invention may be useful in PDT to treat infectious diseases. For example, Cutaneous leishmaniasis and sub-cutaneous leishmaniasis, which occurs extensively in the Mediterranean and Mideast regions, is currently treated with arsenic-containing compounds. PDT has been used to reasonable effect recently, at least in one case, on a human subject. The use of compounds and compositions of the present invention are likewise useful, and potentially offer advantages such as ease of synthesis and better spectral absorption properties.
(x) Tissue sealants. Compounds, compositions and methods of the invention may be useful in PDT as tissue sealants in subjects in need thereof. Light-activated tissue sealants are attractive for sealing wounds, bonding tissue, and closing defects in tissue. There are many applications where sutures or staples are undesirable, and use of such mechanical methods of sealing often leads to infection and scarring.
(xi) Neoplastic disease. Compounds, compositions and methods of the invention may be useful in PDT for treating neoplastic diseases or cancers, including skin cancer, lung cancer, colon cancer, breast cancer, prostate cancer, cervical cancer, ovarian cancer, basal cell carcinoma, leukemia, lymphoma, squamous cell carcinoma, melanoma, plaque-stage cutaneous T-cell lymphoma, and Kaposi sarcoma.

Further, in the modern medical field, there are a variety of treatments including magnetic resonance imaging (MRI) for the diagnosis of diseases. Detection of cancer in its early stages should improve the ability to cure eliminate the cancerous tissue. Early diagnosis of precancerous regions and minute cancer are important subject matters in modern cancer treatments. MRI has emerged as a powerful tool in clinical settings because it is noninvasive and yields an accurate volume rendering of the subject. The image is created by imposing one or more orthogonal magnetic field gradients upon the subject or specimen while exciting nuclear spins with radio frequency pulses as in a typical nuclear magnetic resonance (NMR) experiment. After collection of data with a variety of gradient fields, deconvolution yields a one, two, or three dimensional image of the specimen/subject. Typically, the image is based on the NMR signal from the protons of water where the signal intensity in a given volume element is a function of the water concentration and relaxation times. Local variation in these parameters provide the vivid contrast observed in MR images.

MRI contrast agents act by increasing the rate of relaxation, thereby increasing the contrast between water molecules in the region where the imaging agent accretes and water molecules elsewhere in the body. However, the effect of the agent is to decrease both T₁ and T₂, the former resulting in greater contrast while the latter results in lesser contrast. Accordingly, the phenomenon is concentration-dependent, and there is normally an optimum concentration of a paramagnetic species for maximum efficacy. This optimal concentration will vary with the particular agent used, the locus of imaging, the mode of imaging, i.e., spin-echo, saturation-recovery, inversion-recovery and/or various other strongly T₁-dependent or T₂-dependent imaging techniques, and the composition of the medium in which the agent is dissolved or suspended. These factors, and their relative importance are known in the art. See, e.g., Pykett, Scientific American 246: 78 (1982); Runge et al., Am. J. Radiol. 141: 1209 (1983). When MRI contrast agents are used diagnostically, they may be vascularly perfused, enhancing the contrast of blood vessels and reporting on organ lesions and infiltration. However, the labeling of specific tissues for diagnostic radiology remains a difficult challenge for MRI. Efforts to develop cell and tissue-specific MRI image enhancing agents by modifying existing immunological techniques has been the focus of much research in diagnostic radiology. For example, antibodies labeled with paramagnetic ions, generally the gadolinium chelate Gd-DTPA, have been generated and tested for their effects on MRI contrast of tumors and other tissues (U.S. Pat. No. 5,059,415). Unfortunately, the relaxivity of Gd bound to antibodies has been found to be only slightly better than that of unbound Gd-DTPA (Paajanen et al., Magn. Reson. Med 13: 38-43 (1990)).

MRI is generally used to detect ¹H nuclei in the living body. However, MRI is capable of detecting NMR spectrums of other nuclear species, including ¹³C, ¹⁵N, ³¹P, and ¹⁹F. The ¹⁹F is not abundant in the living body. By incorporating isotopes useful in MRI, such as ¹³C, ¹⁵N, ³¹P or ¹⁹F, and particularly ¹⁹F in the compositions provided herein and administering to a subject, the compounds provided herein would accumulate in target tissue, and subsequent MR imaging would produce NMR data with enhanced signal from the targeted tissue or target compositions due to the presence of the accumulated compound with the MRI recognizable isotope, such as ¹⁹F. Thus, the disclosed compounds may be used as image enhancing agents and provide labeling of specific target tissues or target compositions for diagnostic radiology, including MRI.

In some embodiments, a composition of the present invention may be used to detect target cells, target tissue, and/or target compositions in a subject. Any type of cells, tissue, and/or composition (e.g., normal or healthy cells and/or tissue, diseased cells and/or tissue, cancer cells, hyperproliferative cells and/or tissue, benign tumors, malignant tumors, aneurysms, etc.) may be detected in a subject. In some embodiments, a composition of the present invention may be used to detect the presence of target cells, target tissue, and/or target compositions in a subject. When the compounds provided herein are to be used for detection of target tissue or target composition, the compounds may be introduced into the subject and sufficient time may be allowed for the compounds to accumulate in the target tissue and/or to become associated with the target composition. The area of treatment is then irradiated, generally using light of an energy sufficient to cause luminescence (e.g., fluorescence or phosphorescence) of the compound, and the energy used is usually significantly lower than is required for photodynamic therapy treatment. Luminescence is determined upon exposure to light at the desired wavelength, and the amount of luminescence can be correlated to the presence of the compound, qualitatively or quantitatively, by methods known in the art.

In some embodiments, a composition of the present invention may be used to diagnose the presence of an infecting agent and/or the identity of an infecting agent in a subject. The compounds provided herein may be conjugated to one or more ligands specific for an infecting agent, such as an antibody or antibody fragment, that selectively associates with the infecting agent, and after allowing sufficient time for the targeted compound to associate with the infecting agent and to clear from non-target tissue, the compound may be visualized, such as, e.g., by exposing the tissue and/or compound to light of an energy sufficient to cause luminescence of the compound or to cause the generation of heat and/or ultrasonic waves, or by imaging using diagnostic radiology, including MRI. By way of example, any one of the compounds provided herein may be conjugated to an antibody that is targeted against a suitable Helicobacter pylori antigen, and formulated into a pharmaceutical preparation that, when introduced into a subject, releases the conjugated compound to a gastric mucus/epithelial layer where the bacterium is found. After sufficient time for the compound to selectively associate with the target infecting agent, and for any unbound compound to clear from non-target tissue, the subject may be examined to determine whether any Helicobacter pylori is present. This may be done by MRI to detect accumulated compound because of the presence of ¹⁹F substituents, for example, or by irradiating the suspect target area with light of an energy sufficient to cause luminescence of the compound, such as by using fiberoptics, and detecting any luminescence of the targeted compound.

According to some embodiments of the present invention, a compound of the invention may be used as a chromophore (also referred to as photosensitizers or simply sensitizers) in solar cells, including but not limited to high surface area colloidal semiconductor film solar cells (Gratzel cells), as described in, for example, U.S. Pat. Nos. 5,441,827; 6,420,648; 6,933,436; 6,924,427; 6,913,713; 6,900,382; 6,858,158; and 6,706,963.

In some embodiments, a compound of the invention may be used as a chromophore in the light harvesting rods described in U.S. Pat. Nos. 6,407,330 and 6,420,648 (incorporated herein by reference). The light harvesting rod may comprise one or more compound(s) of the invention coupled to one or two adjacent chromophores depending upon the position thereof in the light harvesting rod. Such light harvesting rods may be utilized to produce light harvesting arrays as described in U.S. Pat. No. 6,420,648 and solar cells as described in U.S. Pat. No. 6,407,330.

In some embodiments, a compound of the invention may be useful immobilized to a substrate for making charge storage molecules and information storage devices containing the same, either individually or as linked polymers thereof, either optionally including additional compounds to add additional oxidation states. Such charge storage molecules and information storage devices are known and described in, for example, U.S. Pat. No. 6,208,553 to Gryko et al.; U.S. Pat. No. 6,381,169 to Bocian et al.; and U.S. Pat. No. 6,324,091 to Gryko et al. The bacteriochlorins of the invention may comprise a member of a sandwich coordination compound in the information storage molecule, such as described in U.S. Pat. No. 6,212,093 to Li et al. or U.S. Pat. No. 6,451,942 to Li et al.

The present invention is explained in greater detail in the following non-limiting experimental section.

### EXAMPLES

The labeling/numbering of compounds provided in the examples sections is relevant to the examples section only and may not correspond to the labeling/numbering provided throughout the rest of the present application. Thus, the labeling/numbering of compounds in the examples section is not to be confused with the labeling/numbering of compounds throughout the rest of the application (e.g., in the summary and detailed description sections and claims).

Abbreviations may include: round bottom flask (RBF), dichloromethane (DCM, or CH₂Cl₂), ethyl acetate (EtOAc), hexanes (hex), methanol (MeOH), isopropanol (IPA), diethyl ether (Et₂O), acetic acid (AcOH), 1-2-dichloroethane (1,2-DCE), tetrahydrofuran (THF), dimethylformamide (DMF), 1,8-diazabicyclo[5.4.0]undec-7-ene (DBU), triethylamine (TEA, or Et₃N), cesium carbonate (Cs₂CO₃), sodium sulfate (Na₂SO₄), and silica (SiO₂).

### Example 1: P2-C1 Dyad, Methyl Ester

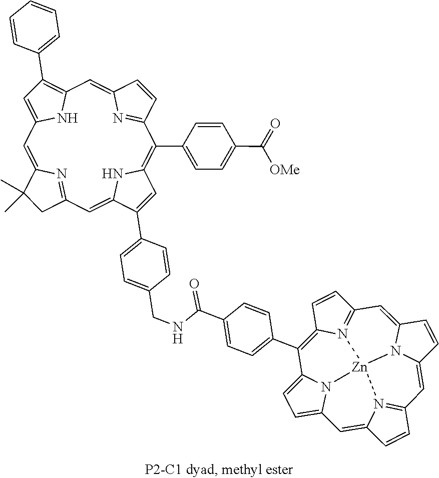

A sample of chlorin C1 (45.0 mg, 72 μmol), porphyrin P2 (57 mg, 80.5 μmol, 1.1 equiv.), Pd(PPh₃)₄ (25 mg, 21.4 μmol), and Cs₂CO₃ (70 mg, 214 μmol) were placed in an oven-dried (50 mL) Schlenk flask equipped with stir bar. The flask was evacuated for 1.0 h and deaerated by three evacuation refill-cycles. The mixture was dissolved by addition of argon purged anhydrous toluene/DMF (24 mL, 2:1) using a syringe filled with argon. The resulting mixture was stirred at 90° C. for 17.0 h. Removed reaction flask from heat and add ˜112 mg of palladium scavenger and stirred for 60 min at room temperature. Washed with EtOAC, brine, dried anhydrous Na₂SO₄, filtered and concentrated. The resulting residue was purified using 12 g SiO₂ column with minimum amount of CH₂Cl₂ and eluted in 0-5% MeOH in CH₂Cl₂ for 20 min to give a dark brown solid in 77 mg (96%) yield.

### Example 2: P2-C1 Dyad, Maleimide

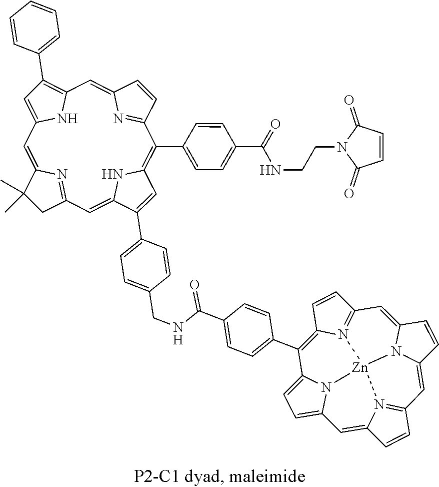

A sample of P2-C1 dyad, methyl ester (77 mg, 68 μmol) in THE (10.0 mL) and MeOH (5.0 mL) was treated with 5N aqueous NaOH (5.0 mL). The reaction mixture was refluxed at 70° C. for 2.0 h. Added 1N aqueous HCl (50.0 mL) and stirred for 10 min. Next, added CH₂Cl₂, washed with brine, dried with anhyd. Na₂SO₄, filtered and concentrated. The resulting P2-C1 dyad carboxylic acid (76 mg, 68 μmol) and TSTU (31 mg, 102 μmol), were dissolved in 17 mL DMF and TEA (83 mg, 820 μmol, 114 μL) was added. The solution was stirred for 1.0 h under argon atmosphere in the dark at RT. Added 2-maleimidethylamine·HCl (18 mg, 102 μmol) and stirred under argon overnight at room temperature. Quenched reaction mixture with CH₂Cl₂, washed with brine, dried over anhydrous Na₂SO₄, filtered, and concentrated. Dissolved residue in minimum amount of CH₂Cl₂ and made silica cake. Eluted in 24 g SiO₂ column with 0-75% EtOAc in hexane for approximately 40 min to give brown solid in 54 mg (64%) yield.

### Example 3: P2-CuC1 Dyad, Maleimide

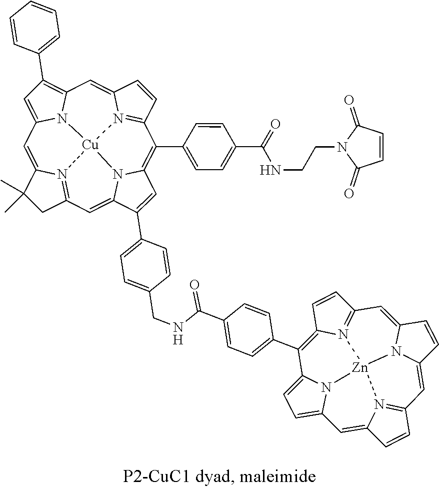

P2-C1 dyad, maleimide (12.6 mg, 10.2 μmol) in anhydrous DMF (4.0 mL) was treated with Cu(OAc)₂ (18.4 mg, 101 μmol). After 2 min of refluxing at 80° C., the brownish solution turns brilliant violet. After 10 min of refluxing, removed reaction flask from heat, and allowed to cool down to RT. Quenched reaction mixture with EtOAc, washed with brine (2×), dried anhydrous Na₂SO₄, filtered, and concentrated. Dissolved residue in minimum amount of CH₂Cl₂ and eluted in 4 g SiO₂ column with 0-90% EtOAc in hexane for approximately 17 min to give dark solid in 10.3 mg (77%) yield.

### Example 4: P3-C1 Dyad

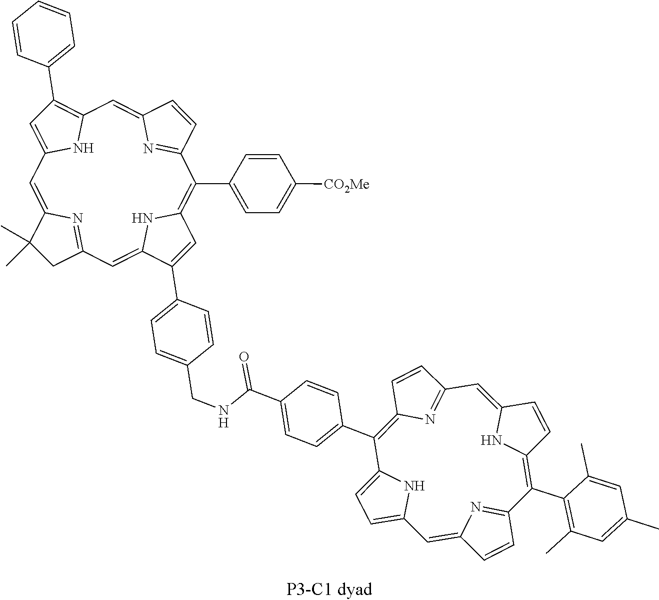

Chlorin C1 (10.0 mg, 16 μmol), porphyrin P3 (14 mg, 19 μmol, 1.2 equiv.), Pd(PPh₃)₄ (6.0 mg, 4.8 μmol), and Cs₂CO₃ (16 mg, 48 mol) were placed in an oven-dried (25 mL) Schlenk flask equipped with stir bar. The flask was evacuated for 1.0 h and deaerated by three evacuation refill-cycles. The mixture was dissolved by addition of argon purged anhydrous tol/DMF (9.0 mL, 2:1) using a syringe filled with argon. The resulting mixture was stirred at 90° C. for 20.5 h. The reaction flask was removed from heat and added 255 mg of palladium scavenger and stirred for 60 min at room temperature. Concentrated and redissolved in minimum amount and eluted in a 12 g SiO₂ column using 0-5% MeOH in CH₂Cl₂ for 20 min to give a dark brown solid in 15.3 mg (81%) yield.

### Example 5: P4-C3 Dyad

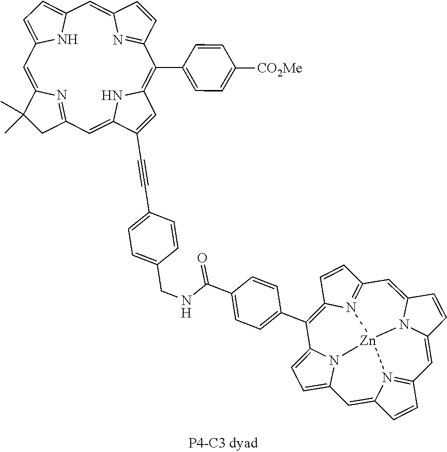

Chlorin C3 (10 mg, 20 μmol), porphyrin P4 (16 mg, 24 μmol) Pd₂(dba)₃ (7.3 mg, 8.0 μmol), and P(o-tol)₃ (7.3 mg, 24 μmol) were placed in an oven-dried (10 mL) Schlenk flask equipped with stir bar. The flask was evacuated for 60 min and deaerated by three evacuation refill-cycles. The mixture was dissolved by addition of argon purged anhydrous toluene/TEA 6 mL, 5:1) using a syringe filled with argon. The resulting mixture was stirred at 60° C. for 19.0 h. Removed reaction flask from heat and added ˜150 mg of palladium scavenger and stirred for 60 min at room temperature. Added EtOAc to reaction mixture (30 mL), washed with brine (30 mL), dried over anhydrous Na₂SO₄, filtered, and concentrated. The residue was dissolved in a minimum amount of CH₂Cl₂ and purified using 12 g SiO₂ column and eluted in 0-2% MeOH in CH₂Cl₂ for 15 min to give a dark red solid in ˜17 mg (76%) yield.

### Example 6: P5-C3 Dyad

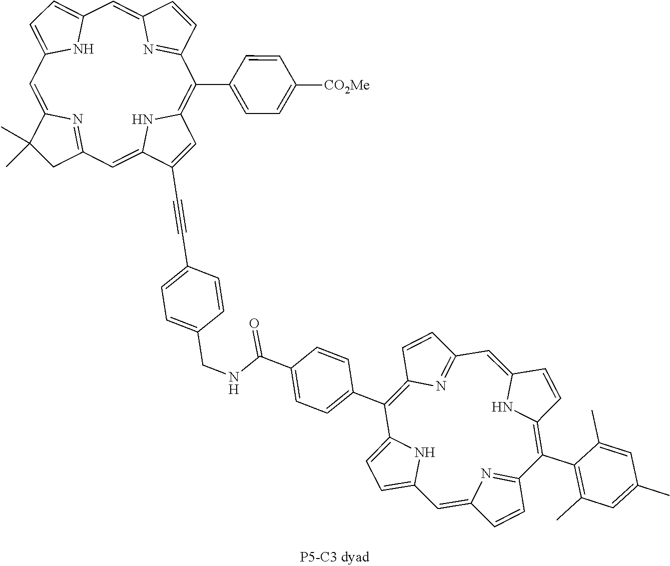

Chlorin C3 (10 mg, 20 μmol), porphyrin P5 (17 mg, 24 μmol), Pd₂(dba)₃ (7.3 mg, 8.0 μmol), and P(o-tol)₃ (7.3 mg, 24 μmol) were placed in an oven-dried (10 mL) Schlenk flask equipped with stir bar. The flask was evacuated for 60 min and deaerated by three evacuation refill-cycles. The mixture was dissolved by addition of argon purged anhydrous toluene/TEA 6 mL, 5:1) using a syringe filled with argon. The resulting mixture was stirred at 60° C. for 19.0 h. Removed reaction flask from heat and add ˜150 mg of palladium scavenger and stirred for 60 min at room temperature. Added EtOAc to reaction mixture (30 mL), washed with brine (30 mL), dried anhydrous Na₂SO₄, filtered, and concentrated. The residue was dissolved in a minimum amount of CH₂Cl₂ and purified using 12 g SiO₂ column and eluted in 0-2% MeOH in CH₂Cl₂ for 15 min to give a dark red solid in ˜17 mg (73%) yield.

### Example 7: P2-C2 Dyad

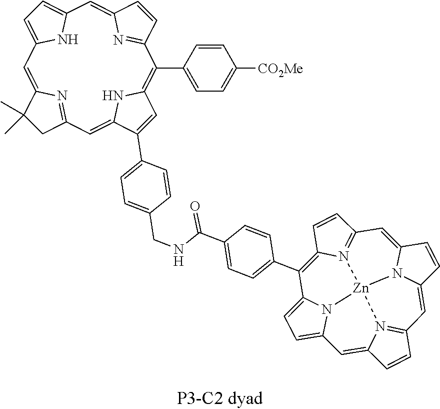

Chlorin C2 (15.0 mg, 27 μmol), porphyrin P2 (23 mg, 33 μmol, 1.2 equiv.), Pd(PPh₃)₄ (9.4 mg, 8.1 μmol), and Cs₂CO₃ (27 mg, 81 μmol) were placed in an oven-dried (50 mL) Schlenk flask equipped with stir bar. The flask was evacuated for 1.0 h and deaerated by three evacuation refill-cycles. The mixture was dissolved by addition of argon purged anhydrous tol/DMF (12 mL, 2:1) using a syringe filled with argon. The resulting mixture was stirred at 90° C. for 17.0 h. Removed reaction flask from heat and added ˜150 mg of palladium scavenger and stirred for 60 min at room temperature. Washed with EtOAC, brine, dried over anhydrous Na₂SO₄, filtered and concentrated. The resulting residue was purified using 12 g SiO₂ column with minimum amount of CH₂Cl₂ and eluted in 0-70% EtOAc in hexane for 33 min to give a brown solid in 22 mg (77%) yield.

### Example 8: P2-ZnC2 Dyad

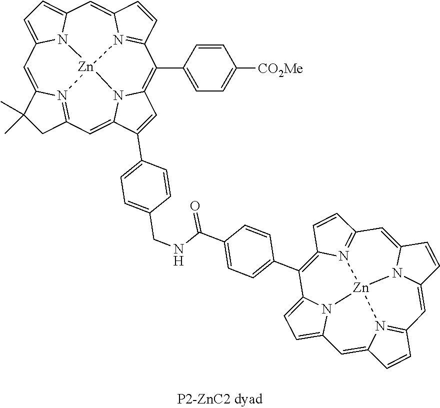

P2-C2 dyad (6.4 mg, 6.1 μmol) in CHCl₃/MeOH (5:1, 6.0 mL) was treated with Zn(OAc)₂ (9.9 mg, 54 μmol, 9.0 equiv). The resulting mixture was refluxed at 50° C. for 60 min, the brownish solution turns violet blue after 10 min of refluxing. Removed reaction flask from heat and allowed to cool to room temperature, concentrated on rotary evaporator, and dissolved the residue in a minimum amount of CH₂Cl₂ and eluted in 4 g SiO₂ column with 0-70% EtOAc in hexane for approximately 20 min to give blue solid in 4.6 mg (68%) yield.

### Example 9: P6-C3 Dyad

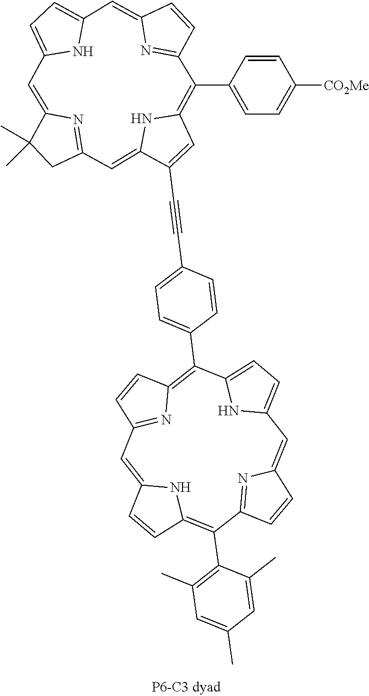 [SMILES: COC(=O)c1ccc(-c2c3nc(cc4ccc(cc5nc(cc6[nH]c2cc6C#Cc2ccc(-c6c7nc(cc8ccc([nH]8)c(-c8c(C)cc(C)cc8C)c8nc(cc9ccc6[nH]9)C=C8)C=C7)cc2)CC5(C)C)[nH]4)C=C3)cc1]

Chlorin C3 (10 mg, 20 μmol), porphyrin P6 (14 mg, 24 μmol) Pd₂(dba)₃ (7.3 mg, 8.0 μmol), and P(o-tol)₃ (7.3 mg, 24 μmol) were placed in an oven-dried (10 mL) Schlenk flask equipped with stir bar. The flask was evacuated for 60 min and deaerated by three evacuation refill-cycles. The mixture was dissolved by addition of argon purged anhydrous toluene/TEA 6 mL, 5:1) using a syringe filled with argon. The resulting mixture was stirred at 60° C. for 19.0 h. Removed reaction flask from heat and added ˜50 mg of palladium scavenger and stirred for 60 min at room temperature. Added EtOAc to reaction mixture (20 mL), washed with brine (20 mL), dried over anhydrous Na₂SO₄, filtered, and concentrated. The residue was dissolved in a minimum amount of CH₂Cl₂ and purified using 12 g SiO₂ column and eluted in 0-40% EtOAc in CH₂Cl₂ for 30 min to give a brown solid in ˜3.5 mg (18%) yield.

### Example 10: P7-C2 Dyad

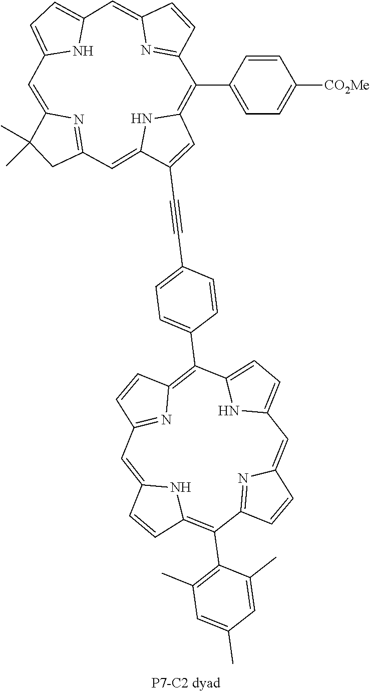 [SMILES: COC(=O)c1ccc(-c2c3nc(cc4ccc(cc5nc(cc6[nH]c2cc6C#Cc2ccc(-c6c7nc(cc8ccc([nH]8)c(-c8c(C)cc(C)cc8C)c8nc(cc9ccc6[nH]9)C=C8)C=C7)cc2)CC5(C)C)[nH]4)C=C3)cc1]

Chlorin C2 (15 mg, 27 μmol), porphyrin P7 (˜16 mg, 30 μmol, 1.1 equiv), Pd₂(dba)₃ (10 mg, 11.0 μmol), and P(o-tol)₃ (10 mg, 33 μmol) were placed in an oven-dried (25 mL) Schlenk flask equipped with stir bar. The flask was evacuated for 60 min and deaerated by three evacuation refill-cycles. The mixture was dissolved by addition of argon purged anhydrous toluene/TEA 12 mL, 5:1) using a syringe filled with argon. The resulting mixture was stirred at 60° C. for 18.0 h. Removed reaction flask from heat and added ˜75 mg of palladium scavenger and stirred for 60 min at room temperature. Added EtOAc to reaction mixture (30 mL), washed with brine (30 mL), dried anhydrous Na₂SO₄, filtered, and concentrated. The residue was dissolved in a minimum amount of CH₂Cl₂ and purified using 12 g SiO₂ column and eluted in 0-60% EtOAc in hexane for 25 min to give a brown solid in ˜20 mg (75%) yield.

### Example 11: P8-C2 Dyad, Methyl Ester

Porphyrin P8 (˜57 mg, 119 μmol, 1.1 equiv), chlorin C2 (60 mg, 108 μmol), Pd₂(dba)₃ (40 mg, 43.0 μmol), and P(o-tol)₃ (40 mg, 130 μmol) were placed in an oven-dried (50 mL) Schlenk flask equipped with stir bar. The flask was evacuated for 60 min and deaerated by three evacuation refill-cycles. The mixture was dissolved by addition of argon purged anhydrous toluene/TEA 24 mL, 5:1) using a syringe filled with argon. The resulting mixture was stirred at 60° C. for 18.0 h. Removed reaction flask from heat and add ˜55 mg of palladium scavenger and stirred for 60 min at room temperature. Added EtOAc to reaction mixture, washed with brine, dried over anhydrous Na₂SO₄, filtered, and concentrated. The residue was dissolved in minimum amount of CH₂Cl₂ to make a silica cake and purified using 24 g SiO₂ column and eluted in 0-50% EtOAc in hexane for 40 min to give a brown solid in 53 mg (51%) yield.

### Example 12: P8-C2 Dyad, Maleimide

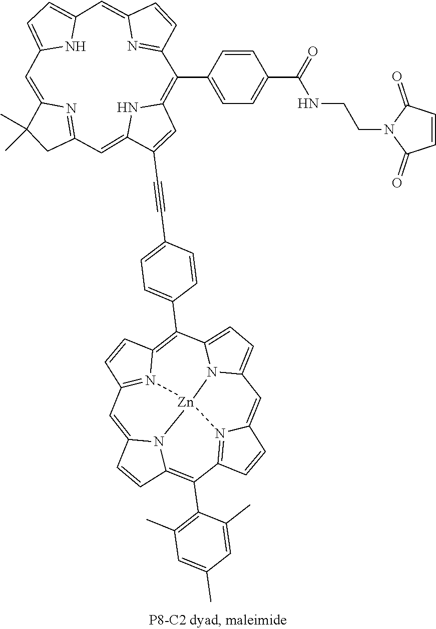

P8-C2 dyad, methyl ester (46 mg, 49 μmol) in THE (10.0 mL), MeOH (5.0 mL) was treated with 5N aqueous NaOH (5.0 mL). The reaction mixture was reflux at 70° C. for 2.0 h. Added 1N aqueous HCl (50.0 mL) and stirred for 10 min. Next, add EtOAc, washed with brine, dried anhydrous Na₂SO₄, filtered and concentrated. Used as it is for next step. The P8-C2 dyad acid (45 mg, 49 μmol), TSTU (22 mg, 73 μmol), and TEA (30 mg, 292 μmol, 41 μL) were dissolved in 12 mL DMF and stirred for 1.0 h under argon atmosphere in the dark at RT. Added 2-maleimidethylamine hydrochloride (13 mg, 73 μmol) and stirred under argon overnight at room temperature. Quenched reaction mixture with CH₂Cl₂, washed with brine, dried over anhydrous Na₂SO₄, filtered, and concentrated. Dissolved residue in minimum amount of CH₂Cl₂ to make a silica cake. Eluted cake in 24 g SiO₂ column with 0-75% EtOAc in hexane for approximately 42 min to give a brown solid in 44 mg (85%) yield.

### Example 13: P8-ZnC2 Dyad

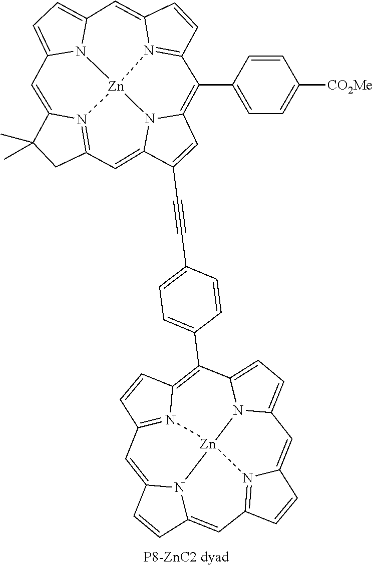

P8-C2 dyad, methyl ester (3.9 mg, 4.1 μmol) in CHCl₃/MeOH (5:1, 3.0 mL) was treated with Zn(OAc)₂ (1.5 mg, 8.1 μmol, 2.0 equiv). The resulting mixture was refluxed at 50° C. for 30 min. The brownish solution turned greenish after 10 min of refluxing. Removed reaction flask from heat, and allowed to cool down to room temperature, concentrated on rotary evaporator and dissolved the residue in a minimum amount of CH₂Cl₂ and eluted in 4 g SiO₂ column with 0-100% EtOAc in hexane for approximately 10 min to give a green solid in 2.6 mg (63%) yield.

### Example 14: P9-C4 Dyad

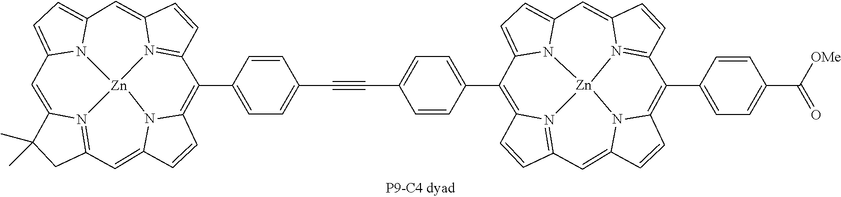

Chlorin C4 (60.0 mg, 107 μmol), porphyrin P9 (78 mg, 129 μmol, 1.2 equiv.) Pd₂(dba)₃ (39 mg, 43.0 μmol), and P(o-tol)₃ (39 mg, 129 μmol) were placed in an oven-dried (50 mL) Schlenk flask equipped with stir bar. The flask was evacuated for 45 min and deaerated by three evacuation refill-cycles. The mixture was dissolved by addition of argon purged anhydrous toluene/TEA 24 mL, 5:1) using a syringe filled with argon. The resulting mixture was stirred at 60° C. for 17.0 h. Removed reaction flask from heat and allowed to cool to room temperature. Added EtOAc to reaction mixture, washed with brine, dried over anhydrous Na₂SO₄, filtered, and concentrated. The residue was dissolved in a minimum amount of CH₂Cl₂, and purified using 24 g SiO₂ column and eluted in 0-100% CH₂Cl₂ in hexane for 27 min and switch to 50% EtOAc in CH₂Cl₂ for 5 min for total of 32 min to give a green purple solid in 68 mg (58%) yield.

### Example 15: P10-C5 Dyad

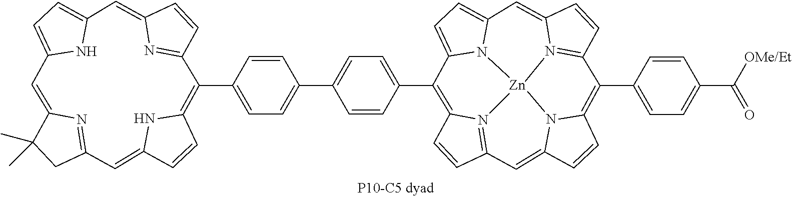

Chlorin C5 (40.0 mg, 81 μmol), porphyrin P10 (63 mg, 89 μmol, 1.1 equiv.) Pd(PPh₃)₄ (28 mg, 24 μmol), and CS₂CO₃ (79 mg, 242 μmol) were placed in an oven-dried (50 mL) Schlenk flask equipped with stir bar. The flask was evacuated for 1.0 h and deaerated by three evacuation refill-cycles. The mixture was dissolved by addition of argon purged anhydrous toluene/TEA 24 mL, 5:1) using a syringe filled with argon. The resulting mixture was stirred at 90° C. for 20 h. Removed reaction flask from hot oil bath and allowed to cool down to room temperature. Added ˜78 mg of palladium scavenger and stirred for 60 min at room temperature. Added EtOAc to reaction mixture, washed with brine, dried anhydrous Na₂SO₄, filtered, and concentrated. The residue was dissolved in minimum amount of CH₂Cl₂ to make a silica cake and purified using 12 g SiO₂ column and eluted in 0-3% MeOH in CH₂Cl₂ for 18 min to give a dark brown solid in 32 mg (40%) yield.

### Example 16: P1-C6 Dyad, Methyl Ester

Chlorin C6 (20.0 mg, 32 μmol), porphyrin P1 (29 mg, 70 μmol, 2.2 equiv.), and PdCl₂(PPh₃)₂ (11.2 mg, 16.0 μmol) were placed in an oven-dried (50 mL) Schlenk flask equipped with stir bar. The flask was evacuated for 1.0 h and deaerated by three evacuation refill-cycles. The mixture was dissolved by addition of argon purged anhydrous toluene/TEA 15 mL, 2:1) using a syringe filled with argon. Next, temperature was raised to 100° C. and reflux for 2.5 h. Removed reaction flask from heat and allowed to cool to room temperature. Added EtOAc to quench, washed with brine, dried over anhydrous Na₂SO₄, filtered, and concentrated. The residue was dissolved in a minimum amount of CH₂Cl₂ and purified using 24 g SiO₂ column and eluted in 0-100% CH₂Cl₂ in hexane for 39 min, then switched to 1% MeOH in CH₂Cl₂ for 8 min for total of 47 min to give a red solid in 41 mg (100%) yield.

### Example 17: P1-C6 Dyad, Maleimide

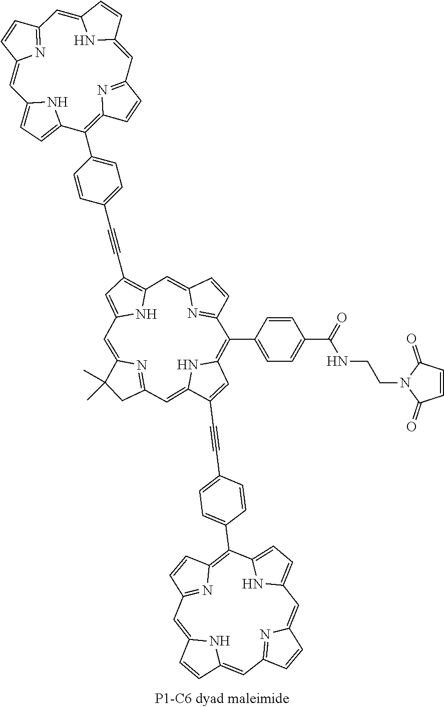 [SMILES: CC1(C)Cc2cc3[nH]c(cc3C#Cc3ccc(-c4c5nc(cc6ccc(cc7nc(cc8ccc4[nH]8)C=C7)[nH]6)C=C5)cc3)c(-c3ccc(C(=O)NCCN4C(=O)C=CC4=O)cc3)c3nc(cc4[nH]c(cc1n2)cc4C#Cc1ccc(-c2c4nc(cc5ccc(cc6nc(cc7ccc2[nH]7)C=C6)[nH]5)C=C4)cc1)C=C3]

A sample of JA28-141 (˜41 mg, 68 μmol) in THE (8.0 mL), MeOH (4.0 mL) was treated with 5N aqueous NaOH (4.0 mL). The reaction mixture was refluxed at 70° C. for 2.0 h. Added 5N aqueous HCl (5.0 mL) and stirred for 10 min. Removed reaction flask from heat and allowed to cool down to room temperature. Diluted with CH₂Cl₂, washed with brine, dried anhyd. Na₂SO₄, filtered and concentrated. Crude carboxylic acid (40 mg, 32 μmol), TSTU (14 mg, 47 μmol), and TEA (19 mg, 190 μmol, 27 μL) was dissolved in 8.0 mL DMF and stirred for 1.5 h under argon atmosphere in the dark at RT. Added 2-maleimidoethylamine·HCl (8.4 mg, 47 μmol) and stirred under argon overnight at room temperature. Added CH₂Cl₂ to reaction mixture, quenched with sat. aq. NaHCO₃, washed with brine, dried over anhydrous Na₂SO₄, filtered, and concentrated. Dissolved residue in a minimum amount of CH₂Cl₂ and eluted on 4 g SiO₂ column with 0-85% EtOAc in hexane for 17 min to give brown solid in 8.0 mg (18%) yield.

### Example 18: P8-C6 Dyad

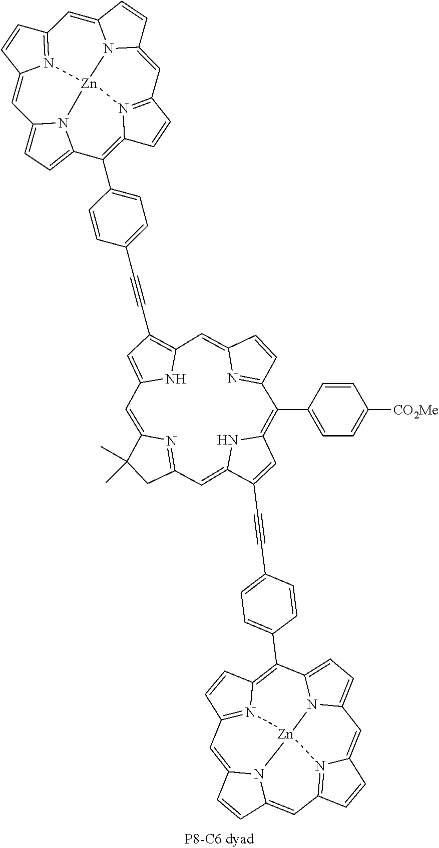

Chlorin C6 (10.0 mg, 16 μmol), porphyrin P8 (16.5 mg, 35 μmol, 2.2 equiv.), and PdCl₂(PPh₃)₂ (2.8 mg, 4.0 μmol) were placed in an oven-dried (25 mL) Schlenk flask equipped with stir bar. The flask was evacuated for 1.0 h and deaerated by three evacuation refill-cycles. The mixture was dissolved by addition of argon purged anhydrous toluene/TEA 9 mL, 2:1) using a syringe filled with argon. The resulting mixture was stirred at 100° C. for 22.5 h. Removed reaction flask from heat and allowed to cool down to room temperature. Added EtOAc to quench, washed with brine, dried anhydrous Na₂SO₄, filtered, and concentrated. The residue was dissolved in a minimum amount of CH₂Cl₂ and purified using 12 g SiO₂ column and eluted in 0-100% CH₂Cl₂ in hexane for 39 min, then switched to 1% MeOH in CH₂Cl₂ for 8 min for total of 47 min to give a red solid in 41 mg (100%) yield.

### Example 19: General Procedure E (P8-BC2a)

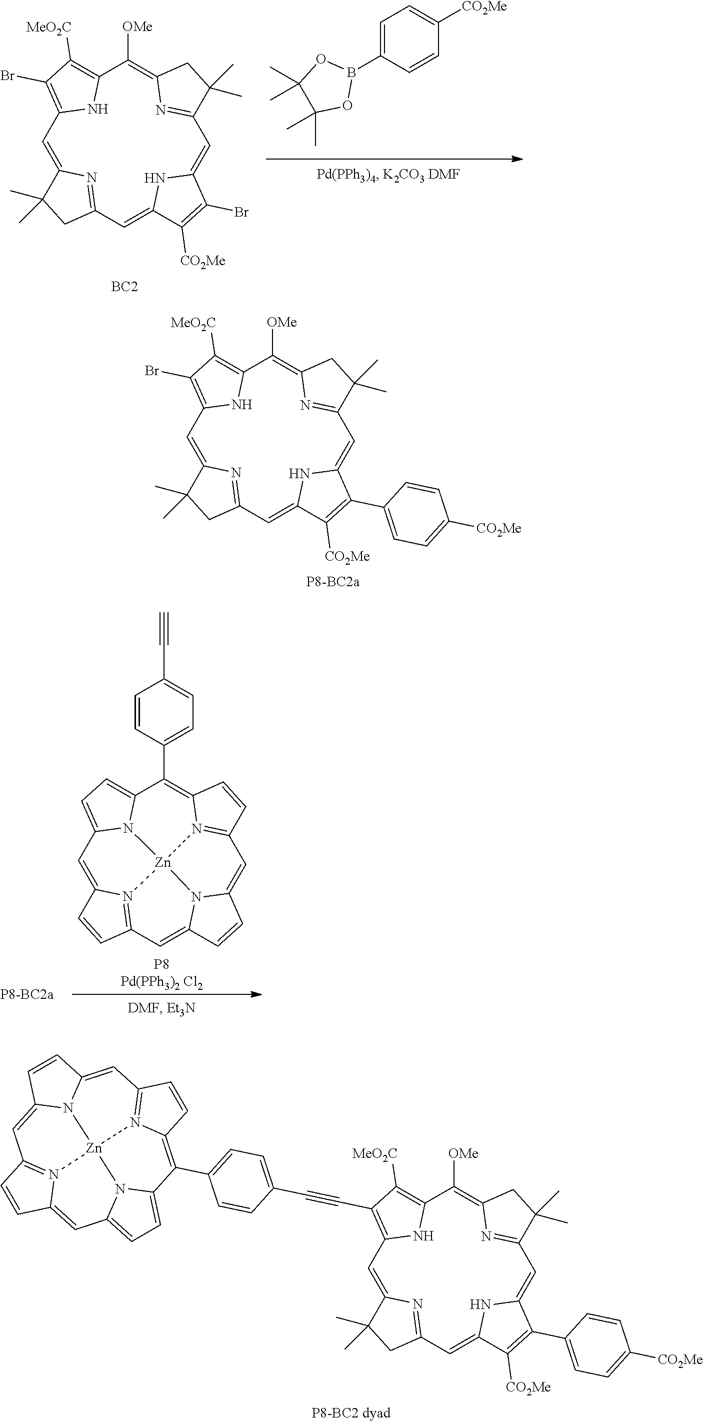

BC2 (0.2 g, 0.3 mmol), 4-methoxycarbonylphenylboronic acid pinacol ester (78 mg, 0.3 mmol), K₂CO₃ (0.41 g, 3 mmol), and Pd(PPh₃)₄ (69 mg, 0.06 mmol) were added to a Schlenk flask and placed under high vacuum for 1 h. Anhydrous DMF and anhydrous toluene were both degassed for 1 h with a strong stream of argon. The flask was deaerated by 3 evacuation/argon refill cycles. Toluene (18 mL) and DMF (9 mL) were added and the flask was placed in a pre-heated oil bath at 80° C. for 16 h. The crude mixture was cooled to RT, diluted with EtOAc, and washed with sat. aq. NaHCO₃. The organic layer was dried, filtered, and concentrated. The mixture was purified by flash chromatography and eluted with a hexanes:EtOAc gradient (69 mg, 32%).

### Example 20: P8-BC2 Dyad 1

Synthesized via General Procedure E. The reaction was conducted using the following amounts: P8-BC2a (89 mg, 0.122 mmol), porphyrin P8 (83 mg, 0.175 mmol), Pd(PPh₃)₂C12 (8.55 mg, 0.0122 mmol), DMF (25 mL), and Et₃N (12.5 mL). The crude product was purified by flash chromatography and eluted with a hexanes:CH₂Cl₂ and a CH₂Cl₂:MeOH gradient. Subsequent purification by Prep TLC gave a red solid in 78% yield.

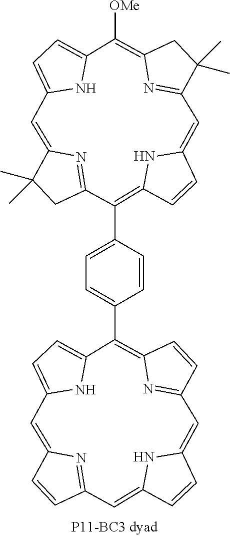

### Example 21: P11-BC3 Dyad

Synthesized via General Procedure E. The reaction was conducted using the following amounts: BC3 (7.2 mg, 15.02 μmol), porphyrin P11 (9.2 mg, 18.02 μmol), K₂CO₃ (10 mg, 72.1 μmol), Pd(PPh₃)₄ (5.2 mg, 4.51 μmol), toluene (2 mL), and DMF (1 mL). The crude product was purified by flash chromatography and eluted with a hexanes:CH₂Cl₂ gradient to give a red/brown solid (6.6 mg, 56%).

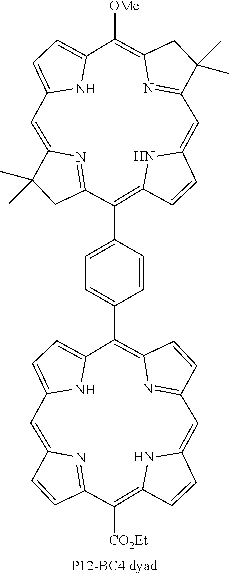

### Example 21: P12-BC4 Dyad

Synthesized via General Procedure E. The reaction was conducted using the following amounts: BC4 (20 mg, 38 μmol), porphyrin P12 (17 mg, 32 μmol), K₂CO₃ (21 mg, 152 μmol), Pd(PPh₃)₄ (11 mg, 9.5 μmol), toluene (4 mL), and DMF (2 mL). The crude product mixture was purified by flash chromatography and eluted with a hexanes:CH₂Cl₂ gradient to give a red brown solid (2.7 mg, 10%).

### Example 22: P11-BC1-1a

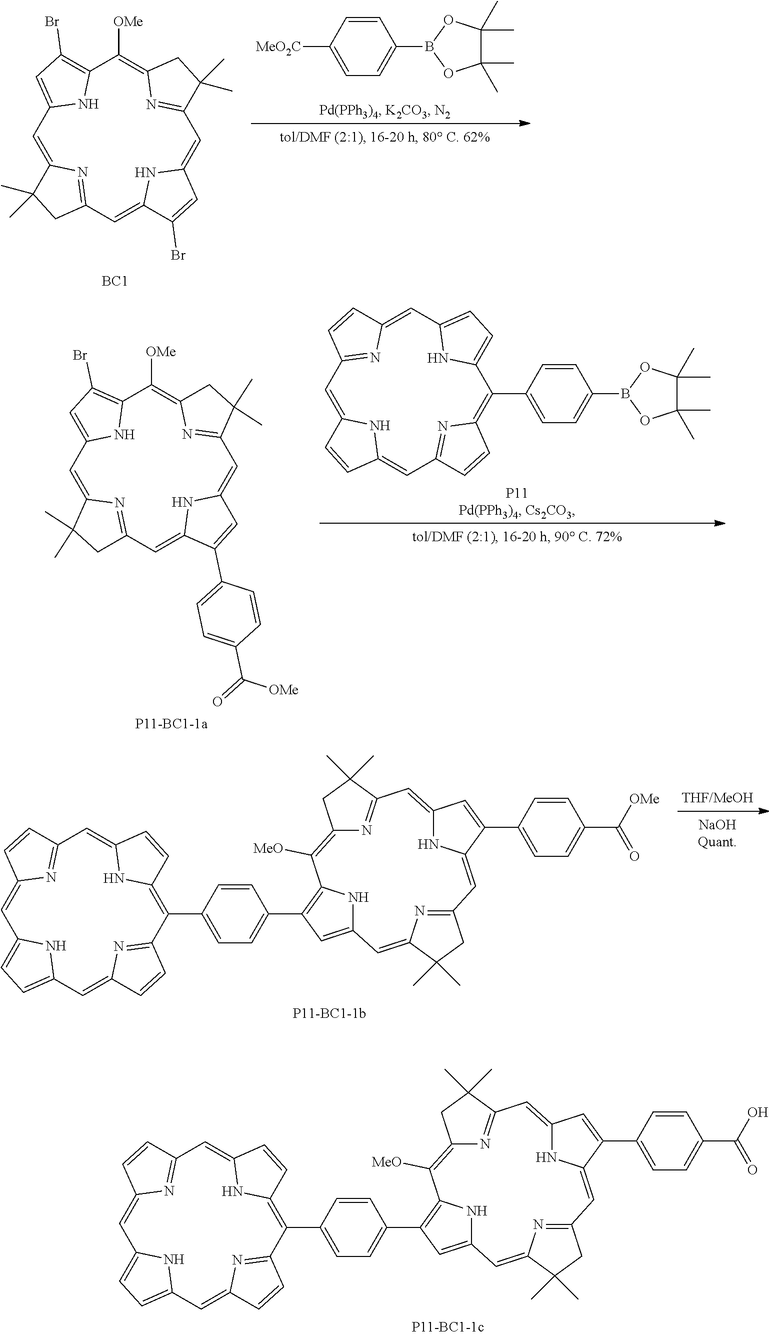

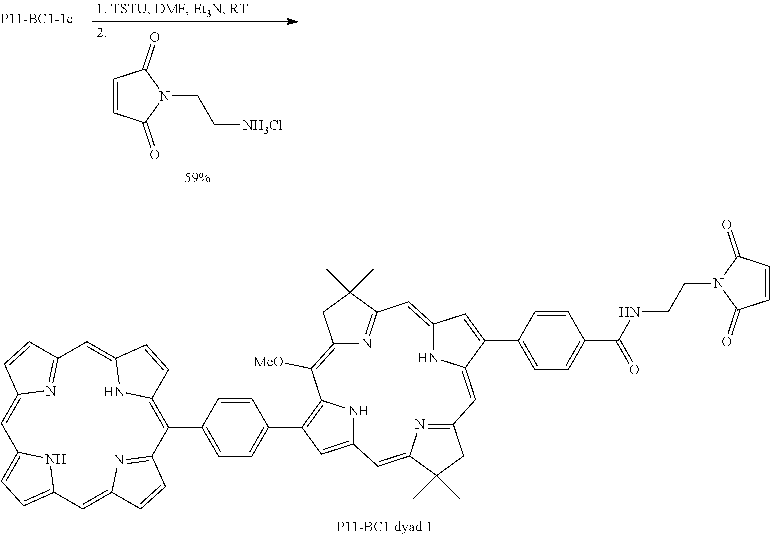

BC1 (50.0 mg, 90 μmol), 4-methoxycarbonylphenylborononic acid (24 mg, 90 μmol), Pd(PPh₃)₄ (20 mg, 18 μmol), and K₂CO₃ (124 mg, 896 μmol) were placed in an oven-dried (50 mL) Schlenk flask equipped with stir bar. The flask was evacuated for 1.0 h and deaerated by three evacuation refill-cycles. The mixture was dissolved by addition of argon purged anhydrous toluene/DMF (15.0 mL, 2:1) using a syringe filled with argon. The resulting mixture was stirred at 80° C. for 17.5 h. Removed reaction flask from heat and allowed to cool to room temperature. Added EtOAc (20 mL), washed with NaHCO₃, brine, dried over anhydrous Na₂SO₄, filtered and concentrated. Redissolved in a minimum amount of CH₂Cl₂ and eluted in a 12 g SiO₂ column using 10-100% CH₂Cl₂ in hexane for 30 min to give green solid in 34 mg (62%) yield.

### Example 23: P11-BC1-1b

Porphyrin P11 (34 mg, 67 μmol, 1.2 equiv), P11-BC1-1a (34 mg, 55 μmol), Pd(PPh₃)₄ (19 mg, 17.0 μmol), and CS₂CO₃ (54 mg, 166 μmol) were placed in an oven-dried (50 mL) Schlenk flask equipped with stir bar. The flask was evacuated for 60 min and deaerated by three evacuation refill-cycles. The mixture was dissolved by addition of argon purged anhydrous toluene/DMF (12 mL, 2:1) using a syringe filled with argon. The resulting mixture was stirred at 90° C. for 18.5 h. Removed reaction flask from heat and allowed to cool to room temperature. Added EtOAc to reaction mixture, washed with NaHCO₃, aqueous brine, dried over anhydrous Na₂SO₄, filtered, and concentrated. The residue was dissolved in a minimum amount of CH₂Cl₂ to make a silica cake and purified using a 12 g SiO₂ column and eluted in 10-100% CH₂C2 in hexane for 28 min to give a dark solid in 37 mg (72%) yield.

### Example 24: P11-BC1-1c

P11-BC1-1b (30 mg, 33 μmol) in THE (8.0 mL), MeOH (4.0 mL) was treated with 5N aqueous NaOH (4.0 mL). The reaction mixture was reflux at 70° C. for 2.0 h. Removed reaction flask from heat and allowed to cool to room temperature. Added 1N aqueous HCl (100.0 mL) and stirred for 10 min. Added CH₂Cl₂, washed with brine, dried over anhydrous Na₂SO₄, filtered and concentrated. Used as it is for next step.

### Example 25: P11-BC1 Dyad 1

P11-BC1-1c (30 mg, 33 μmol), TSTU (15 mg, 49 μmol), and TEA (20 mg, 197 μmol, 27 μL) were dissolved in DMF (8.0 mL) and stirred for 1.5 h under argon atmosphere in the dark at RT. Added 2-maleimidoethylamine hydrochloride (18 mg, 102 μmol) and stirred under argon overnight at room temperature. Quenched reaction mixture with CH₂Cl₂, washed with brine, dried over anhydrous Na₂SO₄, filtered, and concentrated. Dissolved residue in a minimum amount of CH₂Cl₂ and made silica cake. Eluted in 24 g SiO₂ column with 10-100% CH₂Cl₂ in hexane and switched to 75% EtOAc in in hexane for a total of 42 min to give brown solid in 20 mg (59%).

### Example 26: P11-BC1-2a

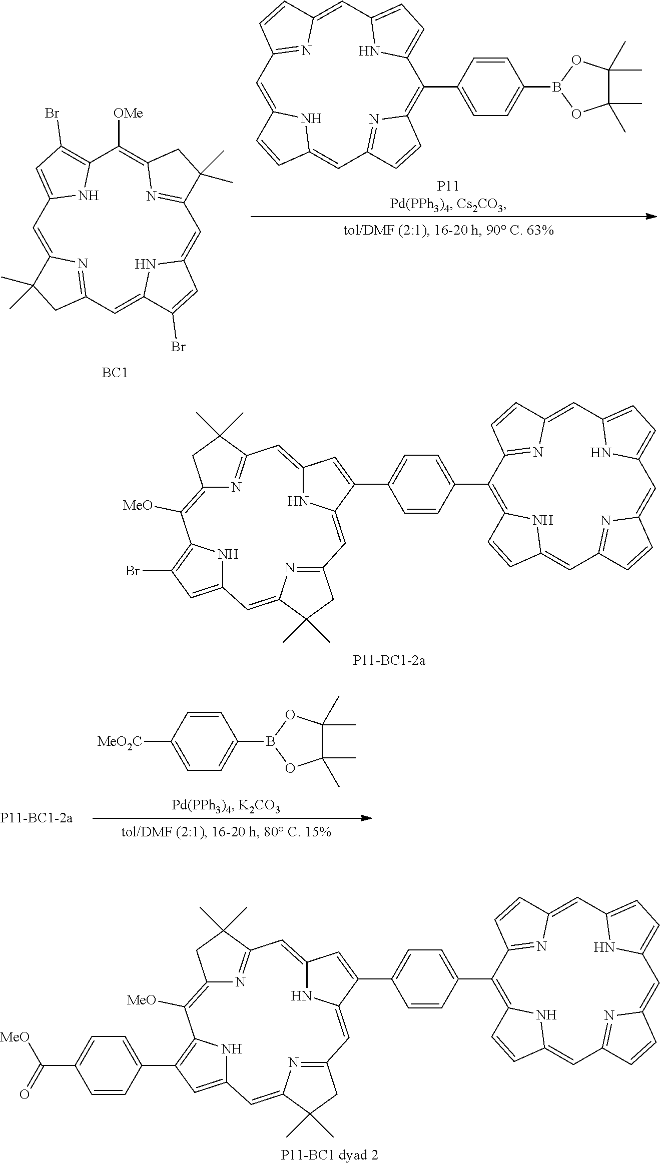

BC1 (30.0 mg, 54 μmol), porphyrin P11 (33.0 mg, 65 μmol, 1.2 equiv.), Pd(PPh₃)₄ (19 mg, 16.0 μmol), and CS₂CO₃ (53 mg, 161 μmol) were placed in an oven-dried (50 mL) Schlenk flask equipped with stir bar. The flask was evacuated for 1.0 h and deaerated by three evacuation refill-cycles. The mixture was dissolved by addition of argon purged anhydrous toluene/DMF (12.0 mL, 2:1) using a syringe filled with argon. The resulting mixture was stirred at 80° C. for 18.5 h. Removed reaction flask from heat and allowed to cool down to room temperature. Added EtOAc (20 mL), washed with NaHCO₃, brine, dried over anhydrous Na₂SO₄, filtered, and concentrated. Redissolved in a minimum amount of CH₂Cl₂ to make a silica cake and eluted in a 12 g SiO₂ column using 10-100% CH₂Cl₂ in hexane for 25 min to give brown solid in 29 mg (63%) yield.

### Example 27: P11-BC1 Dyad 2

P11-BC1-2a (29.0 mg, 34 μmol), 4-methoxycarbonylphenylborononic acid (11 mg, 40 μmol, 1.2 equiv.), Pd(PPh₃)₄ (7.8 mg, 6.7 μmol), and K₂CO₃ (46 mg, 336 μmol) were placed in an oven-dried (50 mL) Schlenk flask equipped with stir bar. The flask was evacuated for 1.0 h and deaerated by three evacuation refill-cycles. The mixture was dissolved by addition of argon purged anhydrous toluene/DMF (12.0 mL, 2:1) using a syringe filled with argon. The resulting mixture was stirred at 80° C. for 18.0 h. Removed reaction flask from heat and allowed to cool to room temperature. Added EtOAc (20 mL), washed with NaHCO₃, brine, dried over anhydrous Na₂SO₄, filtered, and concentrated. Redissolved in a minimum amount of CH₂Cl₂ and eluted in a 12 g SiO₂ column using 10-100% CH₂Cl₂ in hexane for 30 min to give brown solid in 4.7 mg (15%) yield.

### Example 28: P1-BC1a

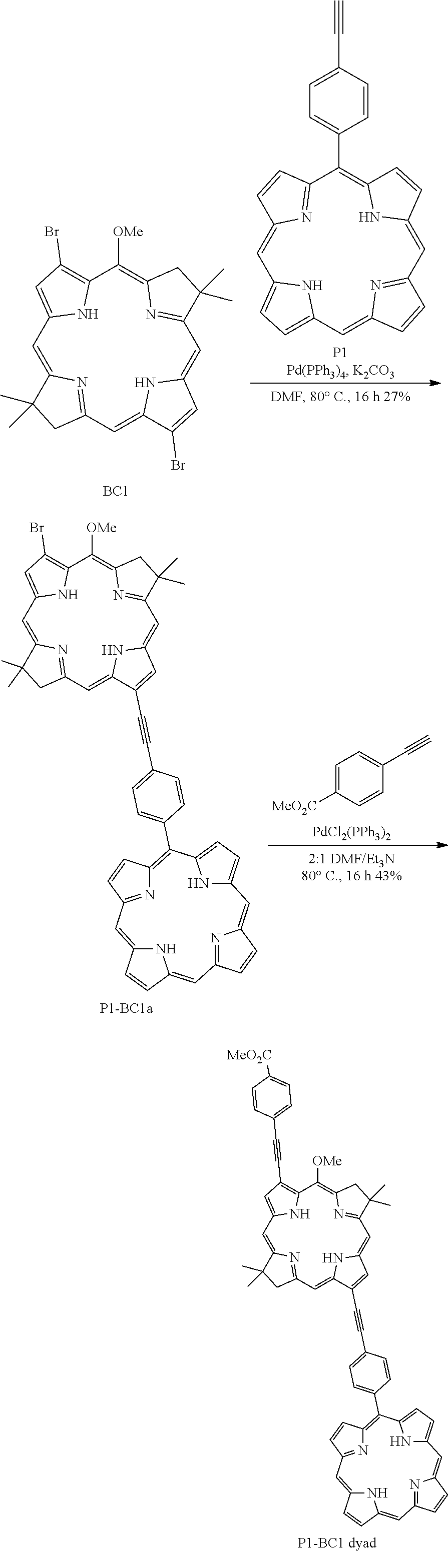

BC1 (55.8 mg, 100.0 μmol), porphyrin P1 (43.1 mg, 105.0 μmol), tetrakis(triphenylphosphine)palladium(0), (11.6 mg, 10.0 μmol), and potassium carbonate (138.2 mg, 1.0 mmol) were added to an oven-dried, vacuum-cooled, Ar flushed 25 mL Schlenk flask with stir bar. The flask was septum sealed, evacuated and Ar flushed (3×). DMF (10.0 mL) was added. The flask was evacuated for 1 min, then Ar flushed and added to a pre-heated oil bath at 80° C. and stirred under low flow Ar. After 16 h, the reaction was cooled, diluted with EtOAc (40 mL) and washed with water (4×50 mL), then brine, and dried over sodium sulfate, filtered, and concentrated. The residue was loaded onto Celite (750 mg, then 150 mg) in DCM and eluted on a 24 g silica column with 10-75% DCM in hexanes. Desired product isolated as 24.3 mg (27%) solid. Abs max (toluene): 380, 405, 516, 740 nm; em max (toluene): 745 nm.

### Example 29: P1-BC1 Dyad

P1-BC1a (20.7 mg, 23.3 μmol), methyl 4-ethynylbenzoate (7.5 mg, 46.6 μmol), and bis(triphenylphosphine)palladium(II) dichloride (1.6 mg, 2.3 μmol) were added to a 10 mL Schlenk flask with stir bar. The flask was evacuated and Ar flushed (3×). DMF (4.8 mL) was added, followed by triethylamine (2.4 mL). The flask was added to a pre-heated oil bath at 80° C. and stirred under low flow Ar. After 16 h, the reaction was cooled and diluted with EtOAc (30 mL). The organic layer was washed with water (4×40 mL) and brine, then dried over sodium sulfate, filtered, and concentrated. Residue loaded onto Celite (300 mg) in DCM and the cake was eluted on a 12 g silica column with 20-90% DCM in hexanes over 15 min. Desired main product isolated as 9.6 mg (43%) brownish red solid. Abs max (toluene): 405, 529, 760; em max (toluene): 765 nm.

### Example 30: P1-BC2 Dyad

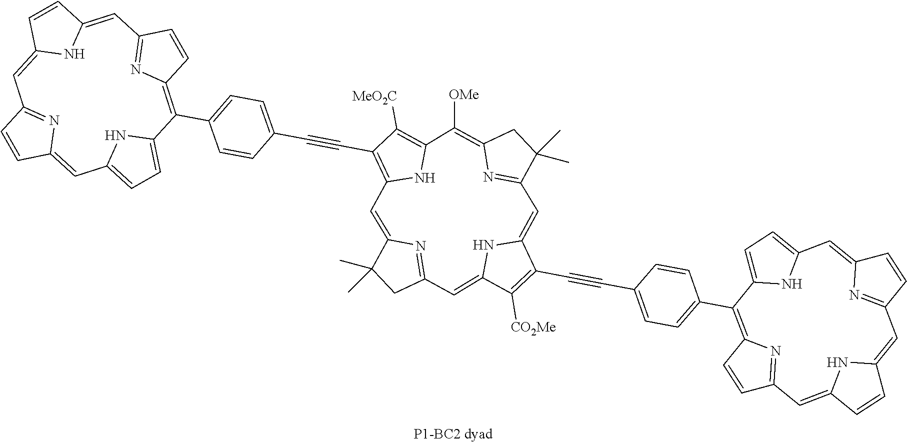 [SMILES: COC(=O)c1c(C#Cc2ccc(-c3c4nc(cc5ccc(cc6nc(cc7ccc3[nH]7)C=C6)[nH]5)C=C4)cc2)c2cc3nc(c(OC)c4[nH]c(cc5nc(cc1[nH]2)CC5(C)C)c(C#Cc1ccc(-c2c5nc(cc6ccc(cc7nc(cc8ccc2[nH]8)C=C7)[nH]6)C=C5)cc1)c4C(=O)OC)CC3(C)C]

BC2 (20.0 mg, 29.7 μmol), porphyrin P1 (26.8 mg, 65.2 mg), and bis(triphenylphosphine)palladium(II) dichloride (6.2 mg, 8.9 μmol) were added to an oven-dried 25 mL Schlenk flask with stir bar. The flask was septum sealed and evacuated for 1 h. Flask was Ar flushed and evacuated/Ar flushed again (2×). DMF (6.0 mL), then triethylamine (3.0 mL) were added. The flask was evacuated while stirring for 1 min, then Ar flushed and added to pre-heated oil bath at 80° C. Stirred under low flow Ar. After 16 h, the reaction was diluted with EtOAc (50 mL) and the organic layer was washed with water (5×50 mL) and brine, then dried over sodium sulfate, filtered, and rinsed with DCM. The sample was loaded onto Celite and eluted with 0-38% EtOAc in hexanes over 10 min, followed by ramp to 80% EtOAc, then 0-3% MeOH in DCM over 8 min. Isolated 21.9 mg (55%) desired product. Abs max (toluene): 405, 499, 795 nm; em max (toluene): 804 nm.

### Example 31: P1d, P8, and P1

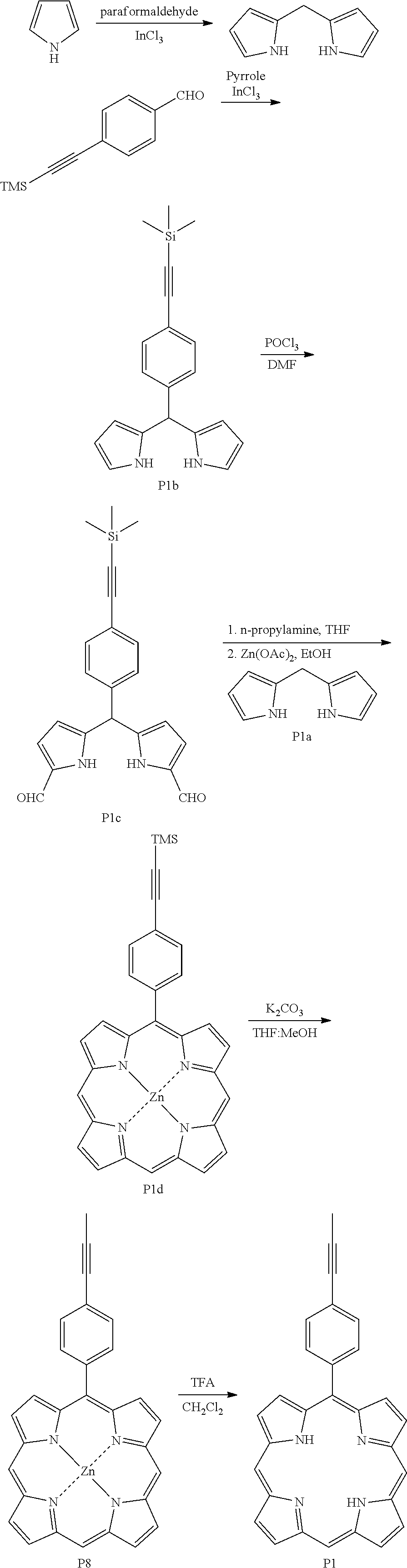

For the synthesis of P1a, see Org. Process Res. Dev. 2003, 7, 799. P1a was isolated as an off-white solid in 58% yield.

For the synthesis of P1b, see Tetrahedron 1994, 50, 11427. The P1b crude product was purified by flash chromatography and eluted with a hexanes:EtOAc gradient to give a thick, orange liquid, which solidified under high vacuum, in 81% yield.

For the synthesis of P1c, see J. Porphyrins Phthalocyanines 2005, 9, 554. P1c was isolated as a thick, brown/orange liquid in 91% yield and was used directly in the next step.

General Procedure A. For P1d, a solution of P1c (0.81 g, 2.16 mmol) in THF (10 mL) was treated with propylamine (3.9 mL, 47.4 mmol) and the solution was stirred at RT for 2 hours. Concentrated to dryness, dried under high vacuum overnight. Isolated (0.99 g, 2.16 mmol, 100%). Step 1 isolated material, P1a (0.32 g, 2.16 mmol), and EtOH (216 mL) were treated with Zn(OAc)₂ (3.96 g, 21.6 mmol). The mixture was refluxed overnight open to air, cooled to RT, and concentrated. The crude product was purified by flash chromatography and eluted with a hexanes:CH₂Cl₂ gradient to give a dark purple solid (0.43 g, 36%).

General Procedure B. For P8, K₂CO₃ (54 mg, 0.39 mmol) was added to a solution of P1d (0.18 g, 0.33 mmol) in THF:MeOH (1:1 v/v, 30 mL) and stirred at RT for 1.5 hours. The mixture was diluted with CH₂Cl₂, washed with H₂O and brine, dried, filtered, and concentrated to give a dark red/purple solid (0.16 g, 100%).

General Procedure C. For P1, Zn porphyrin P8 (0.16 g, 0.33 mmol) was treated with a 20% TFA in CH₂Cl₂ (20 mL) and stirred at RT until complete. The reaction was quenched by slowing pouring the crude mixture into a stirring solution of saturated aq. NaHCO₃. The pH was adjusted to neutral, if necessary, and the layers were separated. The aqueous layer was washed with CH₂Cl₂ and the combined organic layers were washed with brine, dried, filtered, and concentrated. The crude product was purified by flash chromatography and eluted with a hexanes:CH₂Cl₂ gradient to give a purple solid (0.116 g, 86%).

### Example 32: P2c, P2d, and P2

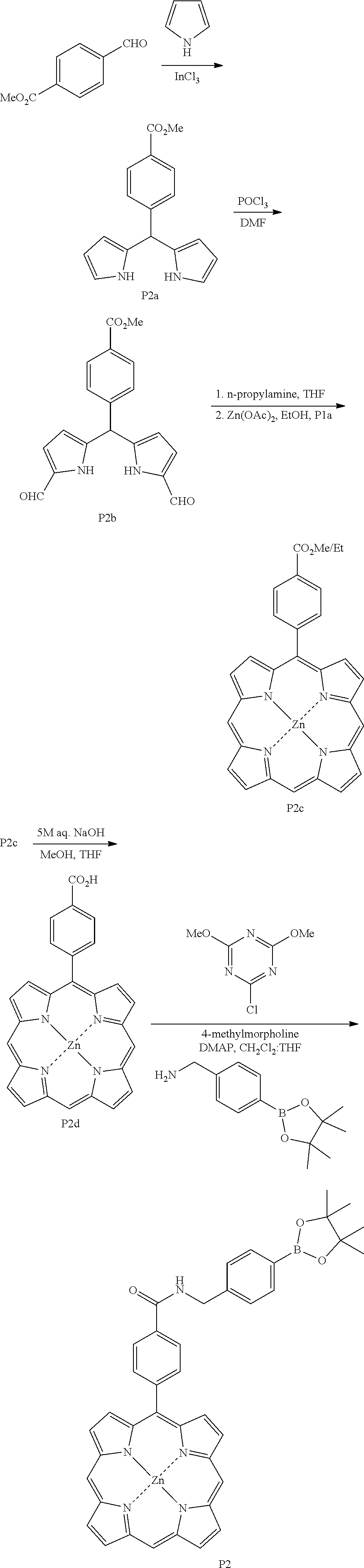

For the synthesis of P2a, see Org. Process Res. Dev. 2003, 7, 799. P2a was isolated as a brown solid in 65% yield.

For the synthesis of P2b, see J. Porphyrins Phthalocyanines 2005, 9, 554. P2b was isolated as a green solid in 44% yield and used directly in the next step.

P2c was synthesized via General Procedure A (See synthesis of P1d). The crude mixture was purified by flash chromatography and eluted with a hexanes:CH₂Cl₂ gradient to give a dark red/purple solid as a mixture of methyl and ethyl esters in 11% yield.

For P2d, a solution of porphyrin P2c (0.39 g, 0.77 mmol), MeOH (5 mL), and THE (10 mL) was treated with 5 M aq. NaOH (5 mL) and refluxed for 1.5 hours. Concentrated to remove MeOH and THE and acidified with 1 M aq. HCl. The precipitate was collected by filtration, air dried, and dried under high vacuum overnight to give a dark red solid (0.34 g, 90%).

General Procedure D. For P2, 4-Methylmorpholine (100 mL, excess) was added to a solution of porphyrin P2d (100 mg, 0.20 mmol) in CH₂Cl₂:THF (3:1 v/v, 20 mL) at RT and stirred for 15 min. 2-Chloro-4,6-dimethoxy-1,3,5-triazine (43 mg, 0.243 mmol) was added and the mixture was stirred at RT for 1.5 hours. After 1.5 hours, 4-dimethylaminopyridine (DMAP, 27 mg, 0.22 mmol), 4-aminomethylphenyl boronic acid pinacol ester (38 mg, 0.21 mmol), and CH₂Cl₂ (16 mL) were added and the mixture was stirred at RT for 1.5 hours. Celite was added to the crude mixture and was concentrated to dryness. The mixture was purified by flash chromatography and eluted with a CH₂Cl₂:MeOH gradient to give a dark red solid (98.6 mg, 69%).

### Example 33: P3b, P3c, P3d, and P3

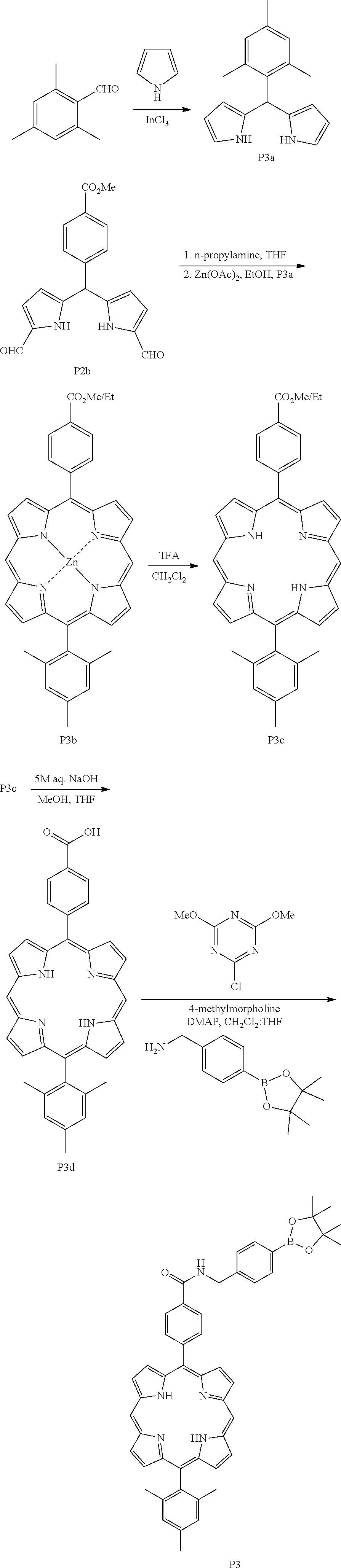

For the synthesis of P3a, see Org. Process Res. Dev. 2003, 7, 799.

P3b was synthesized via General Procedure A. The crude product was purified by flash chromatography and eluted with a hexanes:CH₂Cl₂ gradient to give a dark solid in 18% yield.

P3c was synthesized via General Procedure C. The red solid was isolated in 84% yield and was used directly in the next step.

For the synthesis of P3d, Porphyrin P3c (144.7 mg, 257.2 μmol) was added to 100 mL RBF with stir bar. THE (12.8 mL) was added, followed by MeOH (6.4 mL), and dropwise addition of 5 M aq. NaOH (6.4 mL). Solution was heated to 70° C. for 5 h. Reaction was cooled to room temp and placed in a cool water bath. Added 1 M aq. HCL (27 mL) in portions. EtOAc (35 mL) was added and aq. layer was removed. Organic layer was washed with brine, dried over Na₂SO₄, filtered, and concentrated. Isolated 131.2 mg (93%) dark red solid. MS: [M+H]⁺ calc. 549.3; obs. 549.4.

For the synthesis of P3, porphyrin P3d (60.0 mg, 109.4 μmol) was added to a flame dried 100 mL RBF with stir bar. Flask was evacuated/Ar flushed and 2:1 DCM/THF added (10.9 mL), followed by bulk addition of 4-methylmorpholine (60.1 μL, 546.8 μmol) via pipette direct into solution. After 15 min, 2-chloro-4,6-dimethoxy-1,3,5 triazine (23.0 mg, 131.2 μmol) was added. Reaction was stirred under Ar at room temp. After 1 h, 4-dimethylaminopyridine (14.7 mg, 120.3 μmol) and 4-aminomethylphenyl boronic acid pinacol ester (28.0 mg, 120.3 μmol) were added, followed by additional DCM (10.9 mL). After 2 h, loaded crude reaction mixture onto Celite (540 mg) and eluted cake on 24 g silica column with 0-4% MeOH in DCM over 10 min. Dried product to 67.2 mg (80%). Abs max (toluene): 409, 503 nm; em max (toluene): 633, 702 nm.

### Example 33: P4

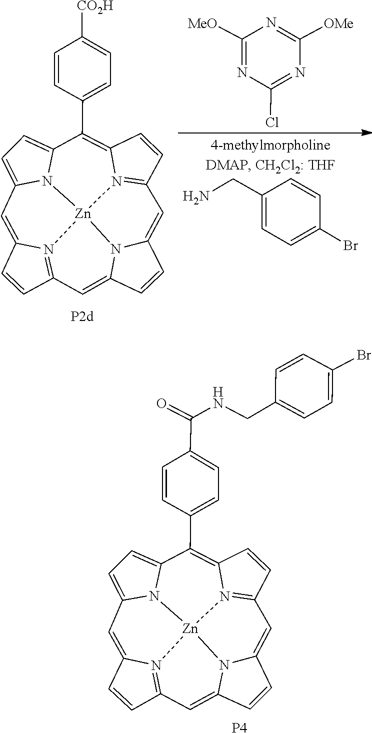

P4 was synthesized via General Procedure D. The crude mixture was purified by flash chromatography and eluted with a CH₂Cl₂:MeOH gradient to give a dark red solid in 90% yield.

### Example 34: P5

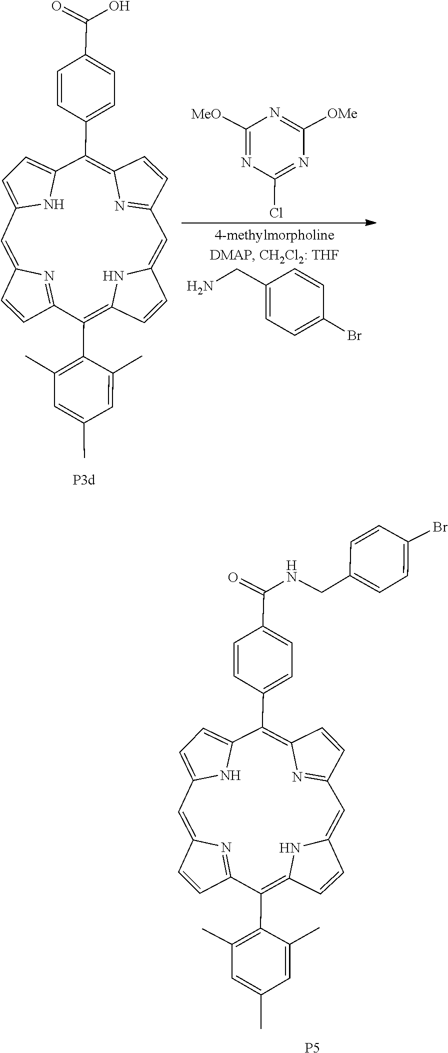

Porphyrin P3d (60.0 mg, 109.4 μmol) was added to flame dried 100 mL RBF with stir bar. Flask evacuated/Ar flushed and 2:1 DCM/THF added (10.9 mL), followed by bulk addition of 4-methylmorpholine (60.1 μL, 546.8 μmol) via pipette direct into solution. After 15 min, 2-chloro-4,6-dimethoxy-1,3,5 triazine (23.0 mg, 131.2 μmol) was added. Reaction was stirred under argon at room temp. After 1 h, 4-dimethylaminopyridine (14.7 mg, 120.3 μmol) and 4-bromobenzylamine (22.4 mg, 120.3 μmol) were added, followed by additional DCM (10.9 mL). After 2 h, loaded crude reaction mixture onto Celite (540 mg) and eluted cake on 24 g SiO₂ column with 0-4% MeOH in DCM over 12 min. Dried product to 69.7 mg (71%, 80% pure).

### Example 35: P6

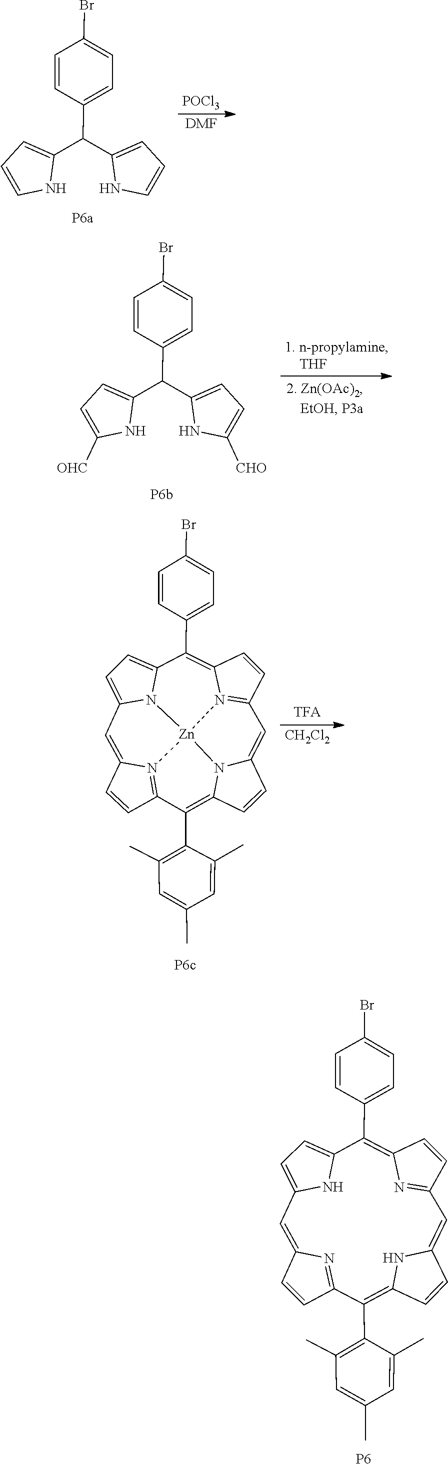

For the synthesis of P6a, see Bioconjugate Chem. 2006, 17, 638. P6a was isolated as a brown solid in 91% yield.

For the synthesis of P6b, see Bioconjugate Chem. 2006, 17, 638. P6b was isolated as a brown foam in 92% yield and used directly in the next step.

P6c was synthesized via General Procedure A. The crude product was purified by flash chromatography and eluted with a hexanes:CH₂Cl₂ gradient to give a red solid in 13% yield.

P6 was synthesized via General Procedure C. The red solid was isolated in 80% yield and was used directly in the next step.

### Example 36: P7

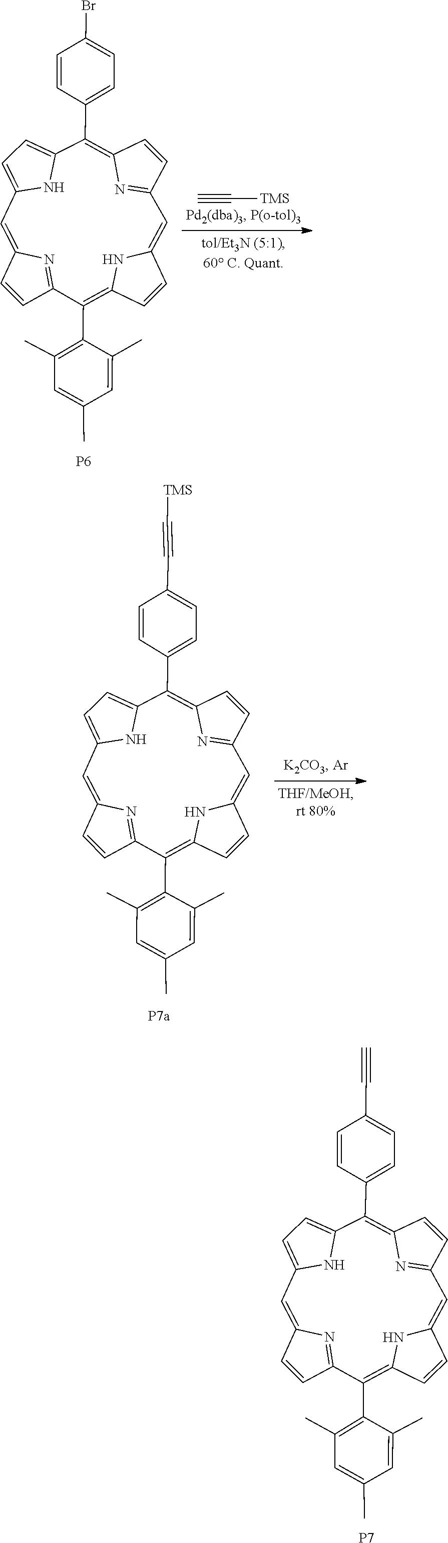

For P7a, porphyrin P6 (50 mg, 86 μmol), Pd₂(dba)₃ (24 mg, 26 μmol), P(o-tol)₃ (65 mg, 214 μmol) was placed in an oven dried (25 mL) Schlenk flask equipped with stir bar. The flask was evacuated for 1 h and deaerated by three evacuation refill cycles. The mixture was dissolved by addition of argon purge anhydrous toluene/TEA (12 mL, 5:1) through syringe filled with argon. A sample of trimethylsilylacetylene (36 uL, 257 μmol) was added, and the resulting mixture was stirred at 60° C. overnight. After 18.0 h, the reaction was removed from heat and added EtOAc to quench, washed with brine, dried over anhydrous Na₂SO₄, filtered, and concentrated under high vacuum rotary evaporation to give reddish solid ˜52 mg (100%) yield.

For P7, porphyrin P7a (52 mg, 87 μmol) in THF/MeOH (20 mL, 1:1) was treated with K₂CO₃ (14 mg, 104 μmol) for 30 min. The crude reaction mixture was diluted with CH₂Cl₂, washed with water, brine, extracted, and dried over anhydrous Na₂SO₄, filtered, and concentrated. The residue was purified by dissolving in a minimum amount of CH₂Cl₂ and eluted in a 12 g SiO₂ column using 0-40% CH₂Cl₂ in hexane for 21 min to give dark green solid in 44 mg (80%) yield.

### Example 37: P8

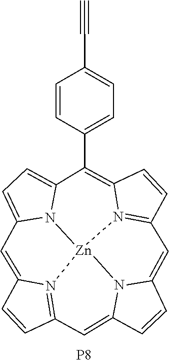

P8 was formed be method described with reference for porphyrin P1.

### Example 38: P9

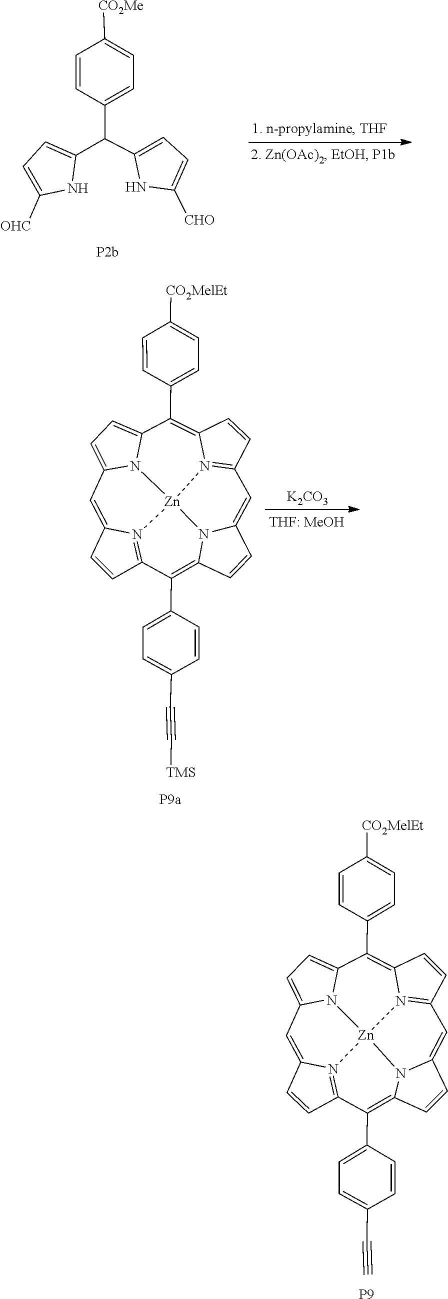

P9a was synthesized via General Procedure A. The crude product was purified by flash chromatography and eluted with a 1:1 hexanes:CH₂Cl₂ to 100% CH₂Cl₂ to 95:5 CH₂Cl₂:MeOH to give a dark red/purple solid as a mixture of methyl and ethyl esters in 40% yield.

P9 was synthesized via General Procedure B. The crude product was purified by flash chromatography and eluted with a hexanes:CH₂Cl₂ gradient to give a red solid in 38% yield.

### Example 39: P10

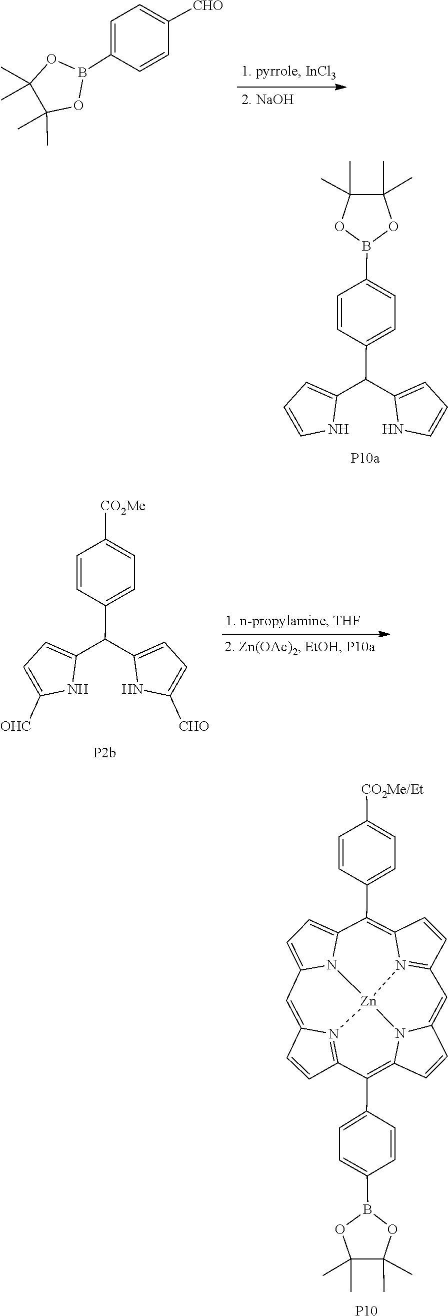

For the synthesis of P10a, see Bioconjugate Chem. 2006, 17, 638. The crude P10a product was triturated with hexanes and the yellow/brown solid was collected by filtration, dried under high vacuum, and isolated in 81% yield.

P10 was synthesized via General Procedure A. The crude product was purified by flash chromatography and eluted with a hexanes:CH₂Cl₂ gradient to give a pink/purple solid as a mixture of methyl and ethyl esters in 8% yield.

### Example 40: P11

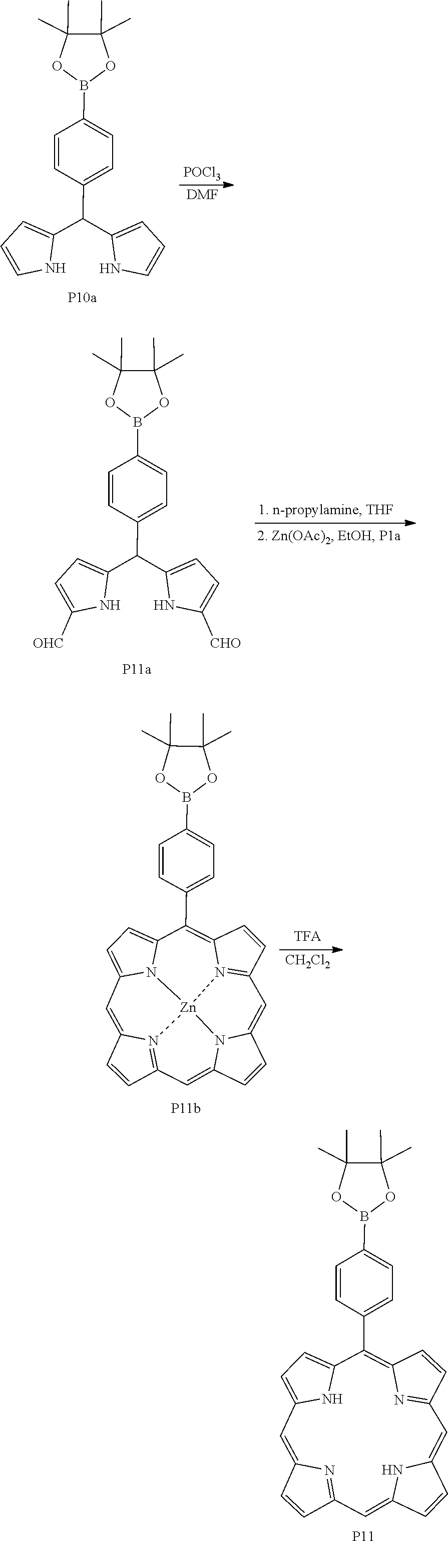

For the synthesis of P11a, see Bioconjugate Chem. 2006, 17, 638. The crude brown foam was isolated in 77% yield and used directly in the next step.

P11b was synthesized via General Procedure A. The crude product was purified by flash chromatography and eluted with a hexanes:CH₂Cl₂ gradient to give a red/brown solid in 11% yield.

P11 was synthesized via General Procedure B. The crude product was purified by flash chromatography and eluted with a hexanes:CH₂Cl₂ gradient to give an orange solid in 49% yield.

### Example 41: P12

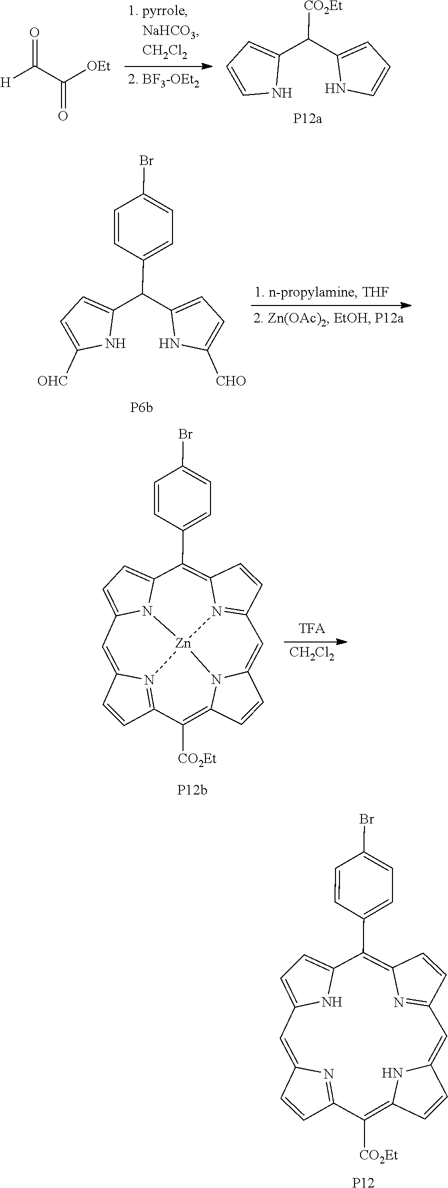

For P12a. see Bioorg. Med. Chem. 2003, 11, 2695. The crude product was purified by flash chromatography and eluted with a hexanes:CH₂Cl₂ gradient to give a thick, light orange liquid, which solidified upon freezing, in 61% yield.

P12b was synthesized via General Procedure A. The crude product was purified by flash chromatography and eluted with a hexanes:CH₂Cl₂ gradient to give a purple solid in 14% yield.

P12 was synthesized via General Procedure C. The dark solid was isolated in 74% yield.

### Example 42: P13

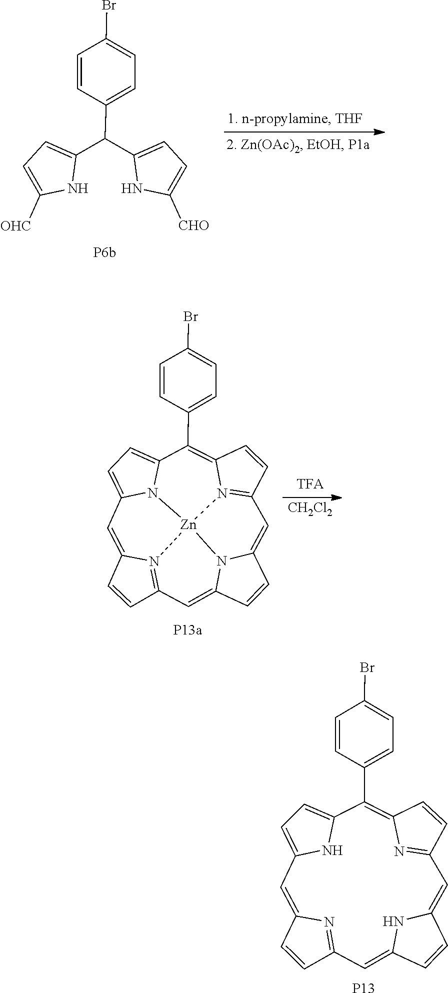

P13a was synthesized via General Procedure A. The crude product was purified by flash chromatography and eluted with a hexanes:CH₂Cl₂ gradient to give a red/brown solid in 17% yield.

P13 was synthesized via General Procedure B. The crude product was purified by flash chromatography and eluted with a hexanes:CH₂Cl₂ gradient to give a purple solid in 59% yield.

### Example 43: C1

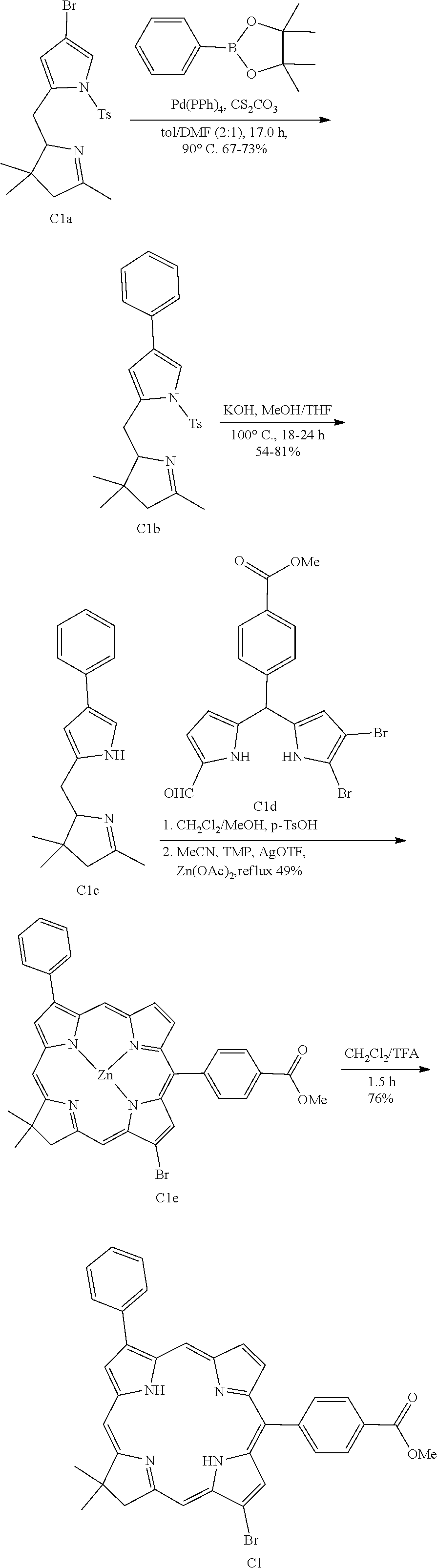

For C1b, compound C1a (prepared as described in J. Porph. Phthal, 2009, 13, 1098-1110) (1.24 g, 2.93 mmol), Ph-Bpin (1.20 g, 5.86 mmol), Pd(PPh₃)₄ (1.02 g, 0.879 mmol), and Cs₂CO₃ (2.86 g, 8.79 mmol) were placed in an oven-dried (200 mL) Schlenk flask equipped with stir bar. The flask was evacuated for 1.5 h and deaerated by three evacuation refill-cycles. The mixture was dissolved by addition of argon purged anhydrous toluene/DMF (60 mL, 2:1) using a syringe filled with argon. The resulting mixture was stirred at 90° C. for 17.0 h. The reaction flask was removed from heat and added ˜112 mg of palladium scavenger and stirred for 60 min at room temperature. Washed with EtOAC, brine, and water, then dried over anhydrous Na₂SO₄, filtered, and concentrated. The resulting residue was purified using 40 g SiO₂ column with minimum amount of CH₂Cl₂ to make a silica cake and eluted in 0-25% EtOAc in hexane for 42 min to give a yellow solid in 0.818 g (67%) yield.

For C1c, compound C1b (818 mg, 1.95 mmol) was dissolved MeOH (37 mL), THE (37 mL). Aqueous KOH (5N, 37 mL) was added and the reaction mixture was refluxed at 100° C. overnight. The reaction flask was removed from heating oil bath and allowed to cool to room temperature. Added EtOAc, washed with water and brine. Extracted the aqueous layer with additional EtOAc (3×). The combined organic layers were dried (Na₂SO₄), filtered, and concentrated, and dried under high vacuum to give 282 mg (54%) compound C1c as a light brown solid.

In General Procedure F (used for Cle), a suspension of compound C1c 0.342 g, 1.28 mmol) and compound C1d (prepared as described in J. Org. Chem, 2013, 78, 10678-10691) (0.598 g, 1.28 mmol) in CH₂Cl₂ (37 mL) was treated with p-toluenesulfonic acid monohydrate (1.22 g, 6.42 mmol) in methanol (8.6 mL) and stirred at room temperature for 40 min. The resulting mixture was treated with 2,2,6,6-tetramethylpiperidine for 5-10 min (2.2 mL, 10 equiv). The reaction mixture was concentrated on rotary evaporator and the resulting brown solid was suspended in acetonitrile (128 mL) and treated with zinc acetate (3.53 g, 19.3 mmol), 2,2,6,6-tetramethylpiperidine (5.4 mL, 25 equiv), and silver trifluoromethanesulfonate (0.99 g, 3.85 mmol). The resulting suspension was refluxed for 16.5 h. Removed flask from heating oil bath and allowed to cool to room temperature. Concentrated on rotary evaporator to form dark solid. Dissolved residue in ethyl acetate (300 mL) and filtered through a bed of silica gel on glass frit and washed with excess EtOAc (300 mL) until the eluant was clear, and concentrated. The residue was redissolved in CH₂Cl₂ and prepared as a silica cake. The cake was eluted on a 40 g SiO₂ column with 0-30% EtOAc in hexanes for 25 min. Combined major green fractions, concentrated, and dried under high vacuum to give a green solid in 439 mg (49%) yield.

For C1, compound C1e (408 mg, 589 μmol) was placed in a round bottom flask. A mixture of CH₂Cl₂ (42.1 mL) and TFA (841 μL) was added and the reaction was stirred at room temperature for 1.5 h. Quenched reaction mixture with saturated aqueous NaHCO₃ (85 mL), washed with brine (85 mL), dried (Na₂SO₄), filtered and concentrated. Recrystallized in MeOH/CH₂Cl₂ (20 mL) to give compound C1 as dark solid powder in (284 mg, 76%).

### Example 44: C2

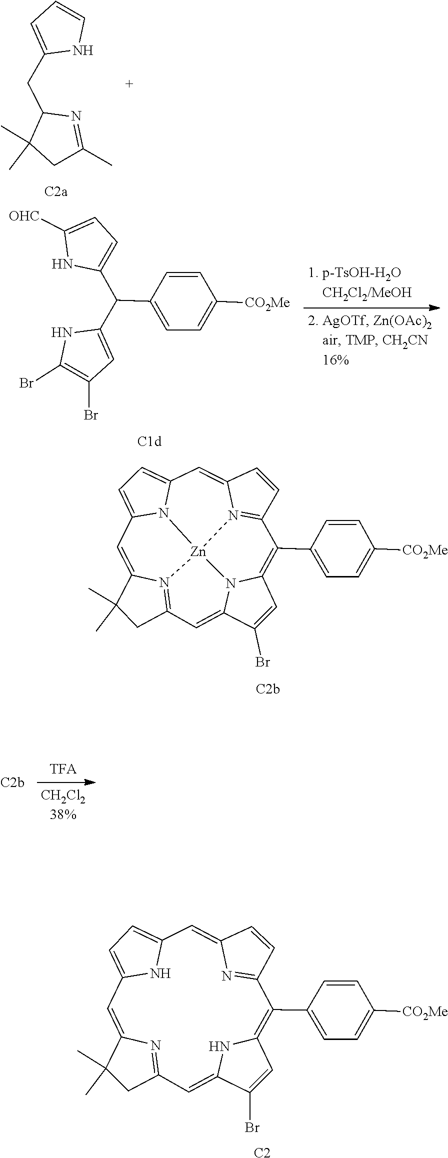

For C2b (prepared as described in J. Org. Chem, 2013, 78, 10678-10691), a solution of p-toluenesulfonic acid hydrate (p-TsOH-H₂O, 4.45 g, 23.4 mmol) in MeOH (32 mL) was added to a suspension of C2a (prepared as described in Org. Process Res. Dev. 2005, 9, 651-659) (0.89 g, 4.68 mmol) and C1d (prepared as described in J. Org. Chem, 2013, 78, 10678-10691) (2.18 g, 4.68 mmol) in DCM (140 mL). The mixture was stirred at RT for 40 min. and 2,2,6,6-tetramethylpiperidine (TMP, 8.77 mL, 52 mmol) was added. The mixture was concentrated to dryness. The residue was suspended in CH₃CN (500 mL) and treated with zinc acetate (Zn(OAc)₂, 12.88 g, 70.2 mmol), silver triflate (AgOTf, 3.61 g, 14.04 mmol), and TMP (20 mL, 119 mmol). The mixture was refluxed overnight, cooled to RT, and concentrated. The crude product was purified by column chromatography with hexanes:EtOAc gradient to give a blue/green solid (0.464 g, 16%).

For General Procedure G (used to prepare C2) (prepared as described in J. Org. Chem, 2013, 78, 10678-10691), zinc chlorin C2b (0.296 g, 0.48 mmol) was treated with a solution of trifluoroacetic acid (TFA, 4 mL) in DCM (50 mL). The solution was stirred at RT for 2 h and was washed with saturated aqueous NaHCO₃ and H₂O. The organic layer was dried, filtered, and concentrated. The residue was purified by column chromatography (silica gel dry load; 24 g column; 100% hexanes to 3:2 hexanes:DCM) to give a green solid (0.1 g, 38%).

### Example 45: C3

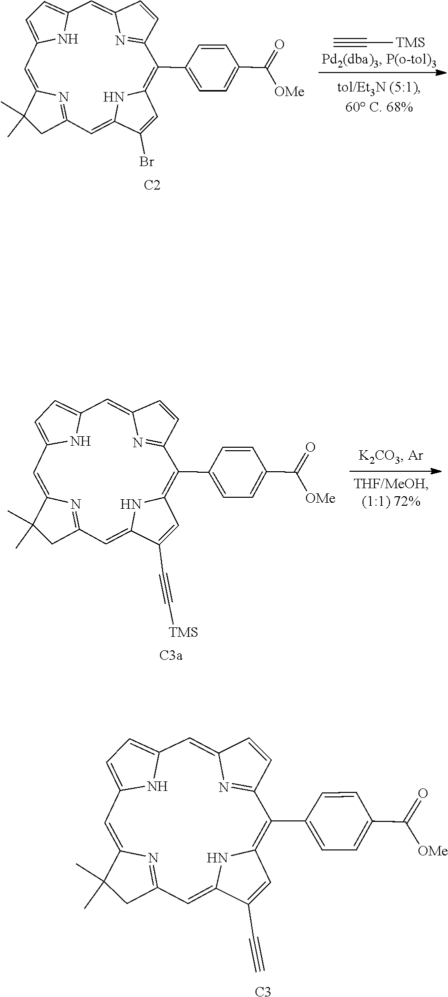

For C3a, chlorin C2 (100 mg, 181 μmol), Pd₂(dba)₃ (50 mg, 54 μmol), and P(o-tol)₃ (138 mg, 452 μmol) were placed in an oven dried (50 mL) Schlenk flask equipped with stir bar. The flask was evacuated for 1 h and deaerated by three evacuation refill cycles. The mixture was dissolved by addition of argon purged anhydrous toluene/TEA (18 mL, 5:1) through syringe filled with argon. A sample of trimethylsilylacetylene (75 uL, 542 μmol) was added, and the resulting mixture was stirred at 60° C. overnight. After 17.5 h, removed from heat and added palladium scavenger (44 mg) and stirred at room temperature for 1 h, Concentrated under high vacuum rotary evaporation and dissolved in a minimum amount of CH₂Cl₂ to make a silica cake. Eluted in a 12 g SiO₂ column using 0-50% hexane in CH₂Cl₂ for 26 min to give green solid in 70 mg (68%) yield.

For C3, chlorin C3a (70 mg, 123 μmol) in THF/MeOH (28 mL, 1:1) was treated with K₂CO₃ (20 mg, 147 μmol) for 30 min. The reaction mixture was diluted with CH₂Cl₂, washed with water (20 mL), brine (20 mL), extracted, dried over anhydrous Na₂SO₄, filtered and concentrated. Dissolved in a minimum amount of CH₂Cl₂ and made a silica cake. Eluted in a 12 g SiO₂ column using 0-40% EtOAc in hexane for 21 min to give dark green solid in 44 mg (72%) yield.

### Example 46: C4

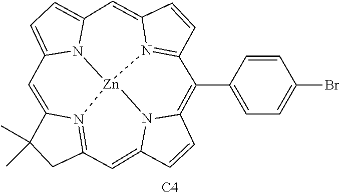

C4 was prepared as described in ChemPhotoChem, 2020, 4, 601-611.

### Example 47: C5

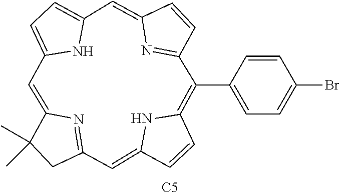

C5 was prepared as described in ChemPhotoChem, 2020, 4, 601-611.

### Example 48: C6

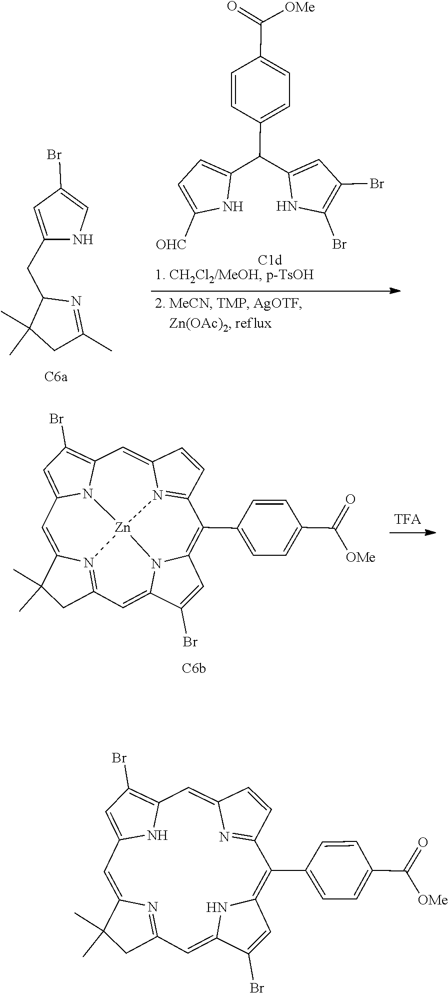

C6b was prepared by General Procedure F from C6a (prepared as described in J. Porph. Phthal, 2009, 13, 1098-1110) and C1d.

C6 was prepared by General Procedure G.

### Example 49: BC1

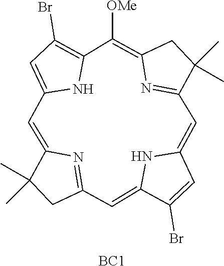

BC1 was prepared as described in J. Org. Chem., 2010, 75, 1016-1039.

### Example 50: BC2

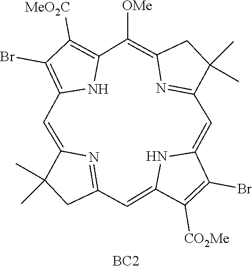 [SMILES: COC(=O)c1c(Br)c2cc3nc(c(OC)c4[nH]c(cc5nc(cc1[nH]2)CC5(C)C)c(Br)c4C(=O)OC)CC3(C)C ; "BC2"]

BC2 was prepared as described in Org. Biomol. Chem., 2014, 12, 86-103.

### Example 51: BC3

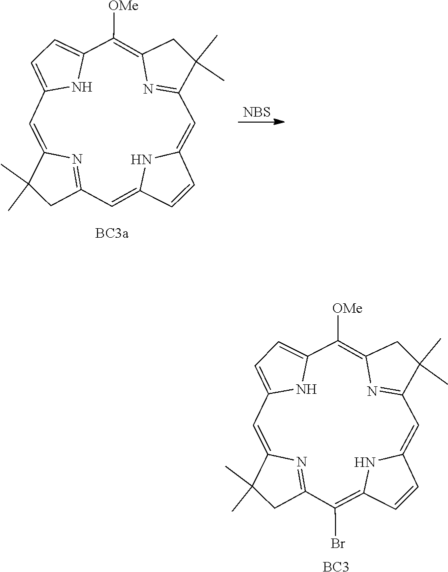

Compound BC3a (prepared as described in J. Org. Chem., 2010, 75, 1016-1039) (40.6 mg, 101.37 mmol) in THE (40 mL) was treated with a solution of NBS (18 mg, 101.37 mmol) in THE (1 mL) at RT. Stirred at RT for 1 h, diluted with CH₂Cl₂ and washed with sat. aq. NaHCO₃. The organic layer was dried, filtered, and concentrated. The crude mixture was purified by flash chromatography and eluted with a hexanes:CH₂Cl₂ gradient to give a green solid (33.8 mg, 70%).

### Example 52: BC4

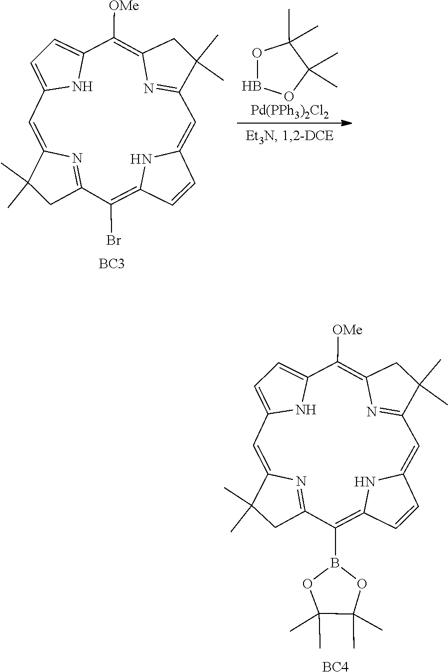

BC4 was prepared as described in Org. Lett., 2016, 18, 4590. Bacteriochlorin BC3 (33.8 mg, 70.5 mmol) and Pd(PPh₃)₂C12 (5 mg, 7.05 mmol) were placed in a Schlenk flask and placed under high vacuum for 1 h. 1,2-Dichloroethane was degassed for 1 h with a stream of argon. After 1 h under high vacuum, the solids were subjected to 3 argon fill/evacuation cycles. 1,2-DCE (14 mL), 4,4,5,5-tetramethyl-1,3,2-dioxaborolane (102 mL, 1420 mmol), and NEt₃ (200 mL, 1420 mmol) were added to the flask. The flask was placed in a pre-heated oil bath and heated at 90° C. for 17 h. After 17 h, the crude mixture was cooled to RT, concentrated, purified by flash chromatography, and eluted with a hexanes:CH₂Cl₂ gradient to give a green solid (33.5 mg, 90%).

### Example 53: Dyad Photophysical Data

The photophysical properties of samples were tested in toluene at room temperature and excited at 405 nm. The results are shown in Table 1.

TABLE 1

| | abs max | abs max | abs max | em max | | ext coeff | ext coeff | QY | brightness | brightness |
|---|---|---|---|---|---|---|---|---|---|---|
| dyad code | B (nm) | Qx (nm) | Qy (nm) | (nm) | FWHM | (abs max B) | (405 nm) | (x405) | (abs max) | (405 nm) |
| P9-C4, | 414 | 540 | 607 | 608 | 14 | 260,700 | 181,500 | 0.04 | 11,570 | 8,055 |
| methyl ester | | | | | | | | | | |
| P2-C1, | 407 | 533 | 652 | 656 | 18 | 374,100 | 346,400 | 0.25 | 94,791 | 87,748 |
| methyl ester | | | | | | | | | | |
| P3-C1 | 410 | 505 | 652 | 656 | 18 | 440,633 | 350,200 | 0.24 | 106,623 | 84,723 |
| P4-C3 | 407 | 532 | 656 | 659 | n/a | 335,300 | 318,900 | 0.07 | 23,813 | 22,648 |
| P5-C3 | 409 | 504 | 657 | 659 | n/a | 214,700 | 171,800 | 0.08 | 17,413 | 13,934 |
| P7-C1; | 410 | 505 | 659 | 660 | 15 | 293,000 | 227,900 | 0.21 | 60,532 | 47,083 |
| P6-C3 | | | | | | | | | | |
| P8-C2, | 408 | 534 | 659 | 661 | 15 | 263,400 | 241,700 | 0.33 | 88,133 | 80,872 |
| methyl ester | | | | | | | | | | |
| P1-C6 | 406 | 499 | 680 | 682 | 17 | 241,800 | 238,800 | 0.34 | 81,579 | 80,567 |
| P11-BC3 | 407 | 511 | 714 | 714 | 15 | 124,800 | 118,100 | 0.17 | 20,998 | 19,871 |
| P11-BC1, | 404 | 514 | 736 | 743 | 25 | 312,500 | 308,300 | 0.24 | 74,793 | 73,788 |
| methyl ester | | | | | | | | | | |
| P1-BC1 | 405 | 529 | 760 | 765 | 21 | 276,800 | 276,800 | 0.23 | 64,405 | 64,405 |
| P8-BC2 | 408 | 538 | 769 | 777 | 29 | 255,600 | 227,600 | 0.20 | 52,298 | 46,569 |
| P1-BC2 | 405 | 499 | 795 | 804 | 31 | 325,400 | 325,400 | 0.19 | 60,845 | 60,845 |

### Example 54: Comparison Porphyrin Photophysical Data

The photophysical properties of samples were tested in toluene at room temperature and excited at 405 nm. The results are shown in Table 2.

TABLE 2

| | abs max | abs max | abs max | em max | | ext coeff | ext coeff | QY | brightness | brightness |
|---|---|---|---|---|---|---|---|---|---|---|
| code | B (nm) | Qx (nm) | Qy (nm) | (nm) | FWHM | (abs | (405 nm) | (x405) | (abs max) | (405 nm) |
| P2c, | 404 | 497 | | 692 | | 245,200 | 237,200 | 0.03 | 8,276 | 8,006 |
| free base | | | | | | | | | | |
| P2c | 407 | 532 | | 621 | | 257,100 | 237,500 | 0.03 | 7,039 | 6,502 |
| P2 | 407 | 533 | | 623 | | 229,200 | 209,100 | 0.03 | 6,466 | 5,899 |
| P3b | 413 | 539 | | 631 | | 257,100 | 98,000 | 0.03 | 8,739 | 3,331 |
| P3c | 409 | 503 | | 699 | | 352,400 | 266,100 | 0.05 | 16,306 | 12,313 |
| P3 | 409 | 503 | | 702 | | 305,400 | 234,100 | 0.04 | 12,560 | 9,628 |

### Example 55: Effect of Linking Groups in P3-C1 and P5-C3 on Photophysical Properties

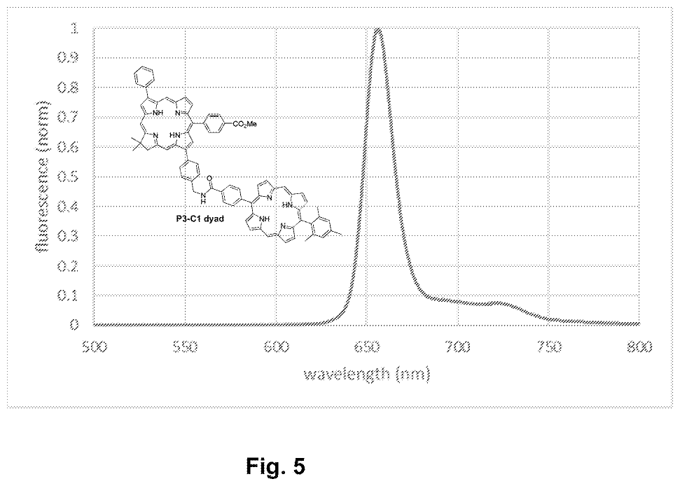

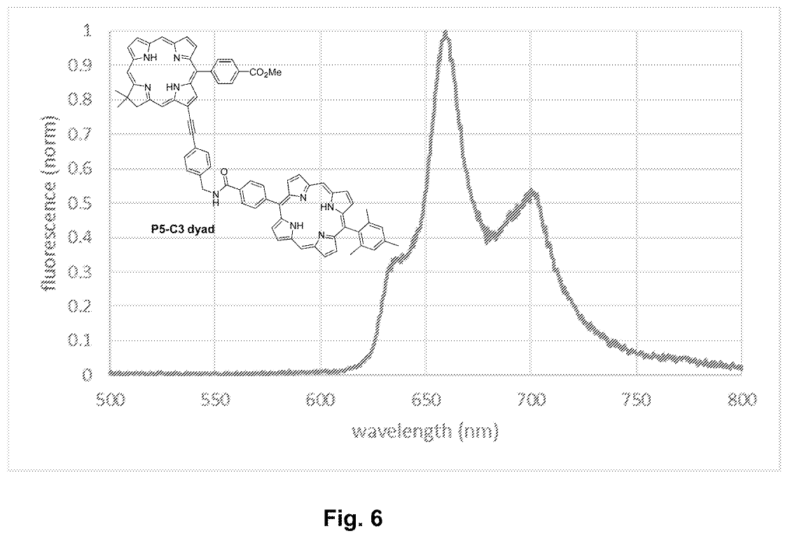

FIG. 5 shows the fluorescence spectrum of compound, P3-C1 (Example 4). P3-C1 shows no residual emission from the porphyrin. FIG. 6 shows the fluorescence spectrum of compound P5-C3 (Example 6). Emission spectra were collected in toluene at room temperature. For P5-C3, porphyrin emission is apparent, indicating reduced energy transfer. P5-C3 contains a more rigid linker between chlorin and porphyrin than in P3-C1, which results in greater spatial separation of the two components and lower energy transfer.

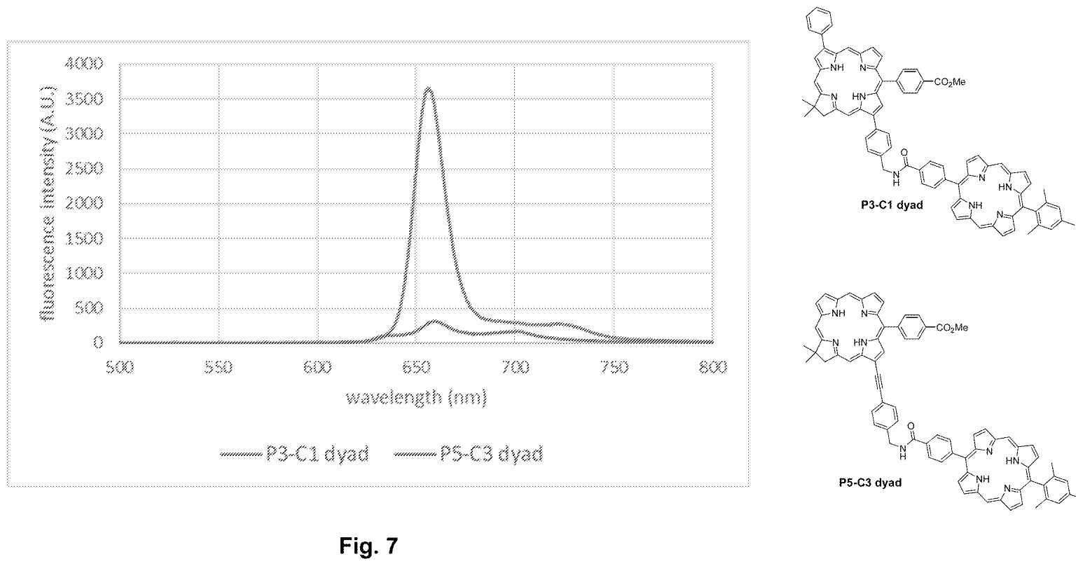

FIG. 7 shows the fluorescence emission spectrum for P3-C1 and P5-C3 at the same sample concentration. Brightness of P3-C1 is significantly increased relative to P5-C3. Brightness (molar absorption coefficient×fluorescence quantum yield): 84,700 (P3-C1), 13,900 (P5-C3).

### Example 56: Effect of Beta vs. Meso Linkage in P11-BC3

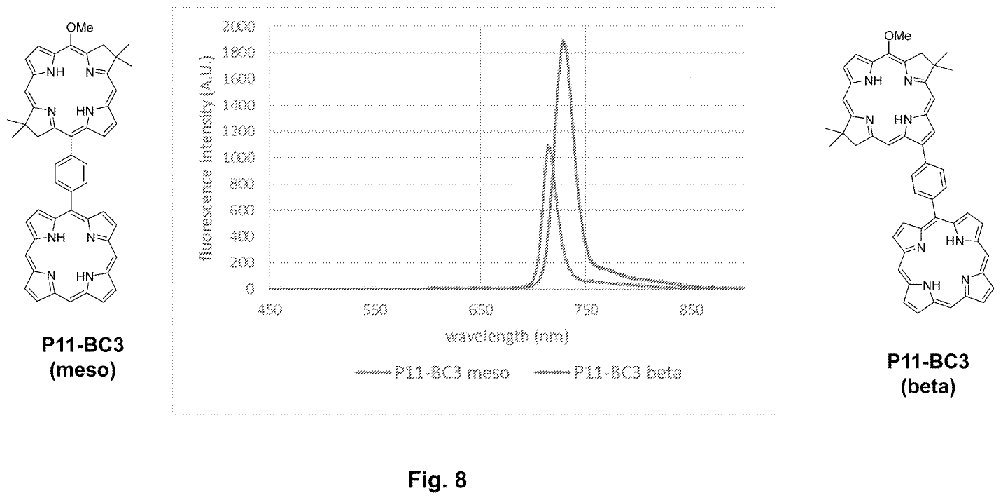

As shown in FIG. 8, without being bound to any particular theory, the effect of the position of the linkage between the porphyrin and the hydroporphyrin may affect the photophysical properties of the compound. Emission spectra were collected using 0.5 uM solutions in toluene. Brightness (molar absorption coefficient×fluorescence quantum yield): 19,900 (P11-BC3 meso), 45,100 (P11-BC1 beta). Thus, the P11-BC1 with the beta linkage produced a significantly brighter emission than P11-BC1 with the meso linkage.

### Example 57: Comparison of Brightness for Hydroporphyrin Monomers vs. Porphyrin-Hydroporphyrin Dyads

Brightness was calculated using an excitation wavelength of 405 nm for a hydroporphyrin monomer having an emission wavelength of approximately 660, 680, 715, or 800 nm and for a fluorescent compound including a porphyrin and a hydroporphyrin structurally similar to the hydroporphyrin monomer. Brightness was calculated as the product of the molar absorption coefficient at 405 nm and the fluorescence quantum yield in toluene at room temperature. Table 3 provides the emission wavelength of the hydroporphyrin monomer and the corresponding dyad, the brightness of the hydroporphyrin monomer excited at 405 nm, the brightness of the dyad excited at 405 nm, and the fold change in brightness of the hydroporphyrin monomer compared to the brightness of the dyad.

TABLE 3

| | abs max | em max | | ext coeff | QY | brightness | fold |
|---|---|---|---|---|---|---|---|
| compound | B (nm) | (nm) | FWHM | (405 nm) | (x405) | (405 nm) | change |
| 660 monomer | 419 | 658 | 19 | 118,200 | 0.30 | 35,700 | |
| P2-C1 dyad | 407 | 656 | 18 | 346,400 | 0.25 | 87,748 | 2.5 |
| 680 monomer | 431 | 682 | 16 | 52,200 | 0.24 | 12,656 | |
| P1-C6 dyad | 406 | 682 | 17 | 238,800 | 0.34 | 80,567 | 6.4 |
| 715 monomer | 372 | 715 | 16 | 2,500 | 0.12 | 306 | |
| P11-BC3 dyad | 407 | 714 | 15 | 118,100 | 0.17 | 19,871 | 64.9 |
| 800 monomer | 385 | 799 | 37 | 29,200 | 0.11 | 3,272 | |
| P1-BC2 dyad | 405 | 804 | 31 | 325,400 | 0.19 | 60,845 | 18.6 |

As shown in Table 3, the brightness of the dyad was significantly increased over the hydroporphyrin monomer alone. Absorbance maxima for the dyads are shifted closer to 405 nm relative to the values for the corresponding monomers. Full width half maximum (FWHM), a measure of emission peak narrowness, is maintained or improved for the dyads in comparison to the structurally related monomer. In some cases, the fluorescence quantum yield for the dyad was greater than that of the corresponding monomer.

The foregoing is illustrative of the present invention and is not to be construed as limiting thereof. The invention is defined by the following claims, with equivalents of the claims to be included therein.

1. A fluorescent compound comprising:
a first porphyrin; and
a first hydroporphyrin;
wherein the first porphyrin is attached to the first hydroporphyrin.

2. The compound of claim 1, wherein the first porphyrin has a structure of one of Formula Ia or Formula Ib: 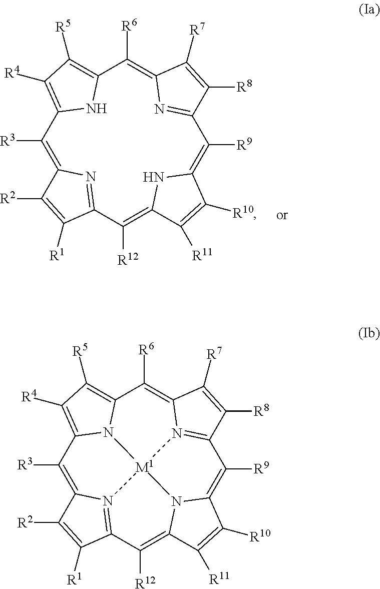
wherein:
R¹, R², R³, R⁴, R⁵, R⁶, R⁷, R⁸, R⁹, R¹⁰, R¹¹, and R¹² are each independently selected from the group consisting of a hydrogen, alkyl, alkenyl, alkynyl, cycloalkyl, cycloalkylalkyl, cycloalkylalkenyl, cycloalkylalkynyl, heterocyclo, heterocycloalkyl, heterocycloalkenyl, heterocycloalkynyl, aryl, aryloxy, arylalkyl, arylalkenyl, arylalkynyl, heteroaryl, heteroarylalkyl, heteroarylalkenyl, heteroarylalkynyl, alkoxy, halo, mercapto, azido, cyano, formyl, carboxylic acid, hydroxyl, nitro, acyl, alkylthio, amino, alkylamino, arylalkylamino, disubstituted amino, acylamino, acyloxy, ester, amide, sulfoxyl, sulfonyl, sulfonate, sulfonic acid, sulfonamide, urea, alkoxylacylamino, aminoacyloxy, hydrophilic groups, linking groups, bioconjugatable groups, surface attachment groups, and targeting groups;
or R³ and R⁵ together represent a fused aromatic or heteroaromatic ring system;
or R⁴ and R⁷ together represent a fused aromatic or heteroaromatic ring system;
or R⁹ and R¹⁰ together represent a fused aromatic or heteroaromatic ring system; or
or R¹⁰ and R¹¹ together represent a fused aromatic or heteroaromatic ring system; and
M¹, if present, is a metal,
wherein at least one of R¹, R², R³, R⁴, R⁵, R⁶, R⁷, R⁸, R⁹, R¹⁰, R¹¹, and R¹² is a direct bond to the first hydroporphyrin or a bond to a linking group that is bonded to the first hydroporphyrin.

3. The compound of claim 1, wherein the first hydroporphyrin is a chlorin or a bacteriochlorin.

4. (canceled)

5. The compound of claim 1, wherein the first hydroporphyrin has a structure of one of Formula IIa-IId: 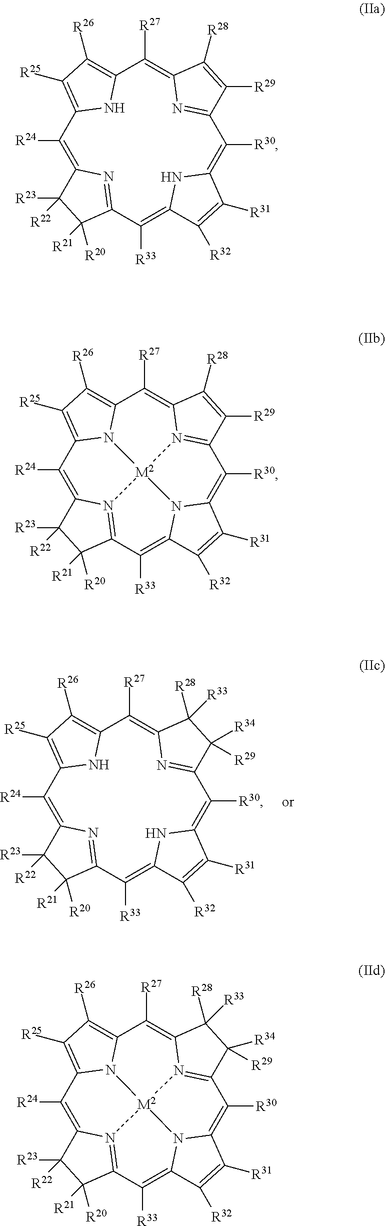
wherein:
R²⁰, R²¹, R²², R²³, R²⁴, R²⁵, R²⁶, R²⁷, R²⁸, R²⁹, R³⁰, R³¹, R³², R³³, and R³⁴ are each independently selected from the group consisting of hydrogen, alkyl, alkenyl, alkynyl, cycloalkyl, cycloalkylalkyl, cycloalkylalkenyl, cycloalkylalkynyl, heterocyclo, heterocycloalkyl, heterocycloalkenyl, heterocycloalkynyl, aryl, aryloxy, arylalkyl, arylalkenyl, arylalkynyl, heteroaryl, heteroarylalkyl, heteroarylalkenyl, heteroarylalkynyl, alkoxy, halo, mercapto, azido, cyano, formyl, carboxylic acid, hydroxyl, nitro, acyl, alkylthio, amino, alkylamino, arylalkylamino, disubstituted amino, acylamino, acyloxy, ester, amide, sulfoxyl, sulfonyl, sulfonate, sulfonic acid, sulfonamide, urea, alkoxylacylamino, aminoacyloxy, hydrophilic groups, linking groups, bioconjugatable groups, surface attachment groups, and targeting groups;
or R²⁰ and R²¹ together are ═O or spiroalkyl;
or R²² and R²³ together are ═O or spiroalkyl;
or R²⁸ and R³³ together are ═O or spiroalkyl;
or R²⁹ and R³³ together are ═O or spiroalkyl;
or R²⁴ and R²⁵ together represent a fused aromatic or heteroaromatic ring system;
or R²⁵ and R²⁶ together represent a fused aromatic or heteroaromatic ring system;
or R²⁶ and R²⁷ together represent a fused aromatic or heteroaromatic ring system;
or R³⁰ and R³¹ together represent a fused aromatic or heteroaromatic ring system; or
or R³¹ and R³² together represent a fused aromatic or heteroaromatic ring system or R³² and R³³ together represent a fused aromatic or heteroaromatic ring system; and
M², if present, is a metal,
wherein at least one of R²⁰, R²¹, R²², R²³, R²⁴, R²⁵, R²⁶, R²⁷, R²⁸, R²⁹, R³⁰, R³¹, R³², R³³, and R³⁴ is direct bond to the first hydroporphyrin or a bond to a linking group that is bonded to the first hydroporphyrin.

6. (canceled)

7. The compound of claim 1, wherein the first porphyrin is attached to the first hydroporphyrin via a direct bond between the first porphyrin and the first hydroporphyrin or wherein the compound further comprises a linking group between the first porphyrin and the first hydroporphyrin that attaches the first porphyrin to the first hydroporphyrin.

8-13. (canceled)

14. The compound of claim 1, wherein the compound is excited at a wavelength in the violet region of the visible light spectrum.

15. (canceled)

16. The compound of claim 1, further comprising a second porphyrin.

17. The compound of claim 16, wherein the first hydroporphyrin is between the first and second porphyrins or wherein the second porphyrin is between the first porphyrin and the first hydroporphyrin.

18. (canceled)

19. The compound of claim 16, wherein the first porphyrin and/or second porphyrin has a structure of Formula Ia and/or the first hydroporphyrin has a structure of Formula IIa or Formula IIc, or wherein the first porphyrin and/or second porphyrin has a structure of Formula Ib and/or the first hydroporphyrin has a structure of Formula IIb or Formula IId.

20-30. (canceled)

31. The compound of claim 1, wherein the compound emits light at a wavelength in the red and/or near-infrared region of the visible light spectrum.

32. (canceled)

33. The compound of claim 1, wherein the compound has a brightness at maximum absorbance in a range of 10,000 M⁻¹cm⁻¹ to 110,000, 200,00, 300,00, 400,000 or 500,000 M⁻¹cm⁻¹, and/or a brightness at an absorbance of 405 nm in a range of 8,000 M⁻¹cm⁻¹ to 90,000, 100,000, 200,00, 300,00, 400,000 or 500,000 M⁻¹cm⁻¹.

34. The compound of claim 1, wherein the compound has an emission intensity for the first porphyrin that is reduced compared to the emission intensity of the first porphyrin alone or the emission intensity when the first porphyrin is absent.

35-39. (canceled)

40. The compound of claim 1, wherein the compound has a molar absorption coefficient and/or fluorescence quantum yield that is greater than the molar absorption coefficient and/or fluorescence quantum yield, respectively, of the first hydroporphyrin alone.

41-46. (canceled)

47. A particle comprising a compound of claim 1.

48-53. (canceled)

54. A composition or kit comprising a compound of claim 1.

55-56. (canceled)

57. A composition or kit comprising:
a first compound having a first absorption and emission spectra comprising a first emission wavelength and
a second compound having a second absorption and emission spectra comprising a second emission wavelength,
wherein the first and second emission wavelengths are different and/or distinct and the first and second compounds are a compound of claim 1.

58-59. (canceled)

60. A method of detecting cells and/or particles using flow cytometry, the method comprising labeling cells and/or particles with a compound of claim 1; and
detecting the compound by flow cytometry, thereby detecting the cells and/or particles.

61. A method of detecting a tissue and/or agent in a subject, the method comprising:
administering to the subject a compound of claim 1; and
detecting the compound within the subject, thereby detecting the tissue and/or agent.

62. A method for treating a cell and/or tissue in a subject in need thereof, the method comprising:
administering a compound of claim 1, and
irradiating the subject or a portion thereof with light of a wavelength and intensity sufficient to treat the cell and/or tissue.

63-64. (canceled)

65. A method of imaging a tissue and/or in a subject, the method comprising:
administering to the subject a compound of claim 1; and
detecting the compound within the subject, thereby imaging the tissue and/or agent.

66-69. (canceled)
